SEQUENCE LISTING (50 sequences)

SEQ ID NO: 1 - protein, 29 aa, Homo sapiens
HSQGTFTSDYSKYLDSRRAQDFVQWLMNT

SEQ ID NO: 2 - protein, 29 aa, Artificial Sequence
  Synthetic Glucagon Analogue
  MOD_RES (16)..(16): Glu, Gln, homoglutamic acid or homocysteic acid
  MOD_RES (17)..(17): Arg, Cys, Orn, homocysteine or acetyl phenylalanine
  MOD_RES (27)..(27): Met, Leu or Nle
HSQGTFTSDYSKYLDXXRAQDFVQWLXNT

SEQ ID NO: 3 - protein, 29 aa, Artificial Sequence
  Synthetic Glucagon Analogue
  MOD_RES (16)..(16): Glu, Gln, homoglutamic acid or homocysteic acid
  MOD_RES (21)..(21): Asp, Cys, Orn, homocysteine or acetyl phenyalanine
  MOD_RES (27)..(27): Met, Leu or Nle
HSQGTFTSDYSKYLDXRRAQXFVQWLXNT

SEQ ID NO: 4 - protein, 29 aa, Artificial Sequence
  Synthetic Glucagon Analogue
  MOD_RES (16)..(16): Glu, Gln, homoglutamic acid or homocysteic acid
  MOD_RES (24)..(24): Gln, Cys, Orn, homocysteine or acetyl phenylalanine
  MOD_RES (27)..(27): Met, Leu or Nle
HSQGTFTSDYSKYLDXRRAQDFVXWLXNT

SEQ ID NO: 5 - protein, 29 aa, Artificial Sequence
  Synthetic Glucagon Analogue
  MOD_RES (16)..(16): Glu, Gln, homoglutamic acid or homocysteic acid
  MOD_RES (21)..(21): Asp, Cys, Orn, homocysteine or acetyl phenyalanine
  MOD_RES (24)..(24): Gln, Cys, Orn, homocysteine or acetyl phenyalanine
  MOD_RES (27)..(27): Met, Leu or Nle
HSQGTFTSDYSKYLDXRRAQXFVXWLXNT

SEQ ID NO: 6 - protein, 29 aa, Artificial Sequence
  Synthetic Glucagon Analogue
  MOD_RES (21)..(21): Asp, Cys, Orn, homocysteine or acetyl phenyalanine
  MOD_RES (27)..(27): Met, Leu or Nle
HSQGTFTSDYSKYLDERRAQXFVQWLXNT

SEQ ID NO: 7 - protein, 29 aa, Artificial Sequence
  Synthetic Glucagon Analogue
  MOD_RES (24)..(24): Gln, Cys, Orn, homocysteine or acetyl phenylalanine
  MOD_RES (27)..(27): Met, Leu or Nle
HSQGTFTSDYSKYLDERRAQDFVXWLXNT

SEQ ID NO: 8 - protein, 29 aa, Artificial Sequence
  Synthetic Glucagon Analogue
  MOD_RES (16)..(16): Glu, Gln, homoglutamic acid or homocysteic acid
HSQGTFTSDYSKYLDXRRAQDFVQWLMNT

SEQ ID NO: 9 - protein, 29 aa, Artificial Sequence
  Synthetic Glucagon Analogue
  MOD_RES (27)..(27): Met, Leu or Nle
HSQGTFTSDYSKYLDERRAQDFVQWLXNT

SEQ ID NO: 10 - protein, 29 aa, Artificial Sequence
  Synthetic Glucagon Analogue
HSQGTFTSDYSKYLDERRAQDFVQWLMNT

SEQ ID NO: 11 - protein, 29 aa, Artificial Sequence
  Synthetic Glucagon Analogue
  MISC_FEATURE: c-term amidation
HSQGTFTSDYSKYLDERRAQDFVQWLMNT

SEQ ID NO: 12 - protein, 29 aa, Artificial Sequence
  Synthetic Glucagon Analogue
  MISC_FEATURE (12)..(16): Lactam ring between side chains at position 12 and 16
HSQGTFTSDYSKYLDERRAQDFVQWLMNT

SEQ ID NO: 13 - protein, 29 aa, Artificial Sequence
  Synthetic Glucagon Analogue
  MISC_FEATURE (16)..(20): Lactam ring between side chains at positions 16 and 20
HSQGTFTSDYSKYLDERRAKDFVQWLMNT

SEQ ID NO: 14 - protein, 29 aa, Artificial Sequence
  Synthetic Glucagon Analogue
  MISC_FEATURE (22)..(24): Lactam ring between side chains at positions 22 and 24
HSQGTFTSDYSKYLDSRRAKDFVEWLMNT

SEQ ID NO: 15 - protein, 29 aa, Artificial Sequence
  Synthetic Glucagon Analogue
  MISC_FEATURE (24)..(28): Lactam ring formed between side chains at positions 24 and 28
HSQGTFTSDYSKYLDSRRAQDFVEWLMKT

SEQ ID NO: 16 - protein, 29 aa, Artificial Sequence
  Synthetic Glucagon Analogue
  MISC_FEATURE (12)..(16): Lactam ring between side chains at positions 12 and 16
  MISC_FEATURE (20)..(24): Lactam ring between side chains at positions 20 and 24
HSQGTFTSDYSKYLDERRAKDFVEWLMNT

SEQ ID NO: 17 - protein, 29 aa, Artificial Sequence
  Synthetic Glucagon Analogue
  MISC_FEATURE (16)..(20): Lactam ring between side chains at positions 16 and 20
  MISC_FEATURE (24)..(28): Lactam ring between side chains at positions 24 and 28
HSQGTFTSDYSKYLDERRADDFVEWLMKT

SEQ ID NO: 18 - protein, 29 aa, Artificial Sequence
  Synthetic Glucagon Analogue
  MISC_FEATURE (16)..(20): Lactam ring between side chains at positions 16 and 20
HSQGTFTSDYSKYLDERRAKDFVEWLMKT

SEQ ID NO: 19 - protein, 30 aa, Artificial Sequence
  Synthetic Glucagon Analogue
  MISC_FEATURE: c-term amidation
  MOD_RES (30)..(30): Variable amino acid
HSQGTFTSDYSKYLDERRAQDFVQWLMNTX

SEQ ID NO: 20 - protein, 29 aa, Artificial Sequence
  Synthetic Glucagon Analogue
  MOD_RES (16)..(16): Ser, Glu, Gln, homoglutamic acid or homocysteic acid
  MOD_RES (20)..(20): Gln, Lys, Arg, Orn or Citrulline
  MOD_RES (24)..(24): Gln or Glu
  MOD_RES (28)..(28): Asn, Asp or Lys
  MOD_RES (29)..(29): Thr or Gly
HSQGTFTSDYSKYLDXRRAXDFVXWLMXX

SEQ ID NO: 21 - protein, 29 aa, Artificial Sequence
  Synthetic Glucagon Analogue
  MISC_FEATURE: c-term amidation
  MOD_RES (2)..(2): Xaa is D-Ser, Ala, Gly, N-methyl Ser or AIB
HXQGTFTSDYSKYLDERRAQDFVQWLMNT

SEQ ID NO: 22 - protein, 29 aa, Artificial Sequence
  Synthetic Glucagon Analogue
  MISC_FEATURE: c-term amidation
  MOD_RES (2)..(2): Xaa is AIB
HXQGTFTSDYSKYLDERRAQDFVQWLMNT

SEQ ID NO: 23 - protein, 29 aa, Artificial Sequence
  Synthetic Glucagon Analogue
  MISC_FEATURE: c-term amidation
  MOD_RES (17)..(17): Cys-PEG
  MOD_RES (27)..(27): Met, Leu or Nle
HSQGTFTSDYSKYLDECRAQDFVQWLXNT

SEQ ID NO: 24 - protein, 29 aa, Artificial Sequence
  Synthetic Glucagon Analogue
  MISC_FEATURE: c-term amidation
  MOD_RES (21)..(21): Cys-PEG
  MOD_RES (27)..(27): Met, Leu or Nle
HSQGTFTSDYSKYLDERRAQCFVQWLXNT

SEQ ID NO: 25 - protein, 29 aa, Artificial Sequence
  Synthetic Glucagon Analogue
  MISC_FEATURE: c-term amidation
  MOD_RES (24)..(24): Cys-PEG
  MOD_RES (27)..(27): Met, Leu or Nle
HSQGTFTSDYSKYLDERRAQDFVCWLXNT

SEQ ID NO: 26 - protein, 11 aa, Artificial Sequence
  Synthetic peptide fragment representing the carboxy terminal 10 amino acids Exendin-4
  MISC_FEATURE (11)..(11): Xaa at position 11 is Lys or Cys
GPSSGAPPPSX

SEQ ID NO: 27 - protein, 9 aa, Artificial Sequence
  Synthetic peptide fragment representing the carboxy terminal 8 amino acids of oxyntomodulin
  MISC_FEATURE (9)..(9): Xaa at position 9 is Lys or Cys
KRNRNNIAX

SEQ ID NO: 28 - protein, 5 aa, Artificial Sequence
  Synthetic peptide
  MISC_FEATURE (5)..(5): Xaa at position 5 is Lys or Cys
KRNRX

SEQ ID NO: 29 - protein, 10 aa, Artificial Sequence
  Synthetic peptide fragment representing the carboxy terminal 10 amino acids of Exendin-4
  MISC_FEATURE: c-term amidation
GPSSGAPPPS

SEQ ID NO: 30 - protein, 31 aa, Artificial Sequence
  Synthetic peptide
  MISC_FEATURE (2)..(2): Xaa is AIB
  MISC_FEATURE (10)..(10): Acylated with a C16 fatty acyl group via gamma- Glu-gamma-Glu spacer
  MISC_FEATURE (16)..(16): Xaa is AIB
  MOD_RES (31)..(31): Xaa is gamma-Glu
  MISC_FEATURE (31)..(31): C-terminal amidation
HXQGTFTSDKSKYLDXRAAQDFVQWLMDTKX

SEQ ID NO: 31 - protein, 31 aa, Artificial Sequence
  Synthetic peptide
  MOD_RES (2)..(2): Xaa is AIB
  MOD_RES (20)..(20): Xaa is AIB
  MISC_FEATURE (24)..(24): Covalently bound to PEG
  MISC_FEATURE (31)..(31): Xaa is gamma-Glu
  MISC_FEATURE (31)..(31): C-terminal amidation
HXQGTFTSDYSKYLDERRAXEFVCWLLDTKX

SEQ ID NO: 32 - protein, 31 aa, Artificial Sequence
  Synthetic peptide
  MOD_RES (2)..(2): D-Ser
  MISC_FEATURE (10)..(10): Acylated with a C16 fatty acyl group via gamma- Glu spacer
  MISC_FEATURE (31)..(31): Xaa is gamma-Glu
  MISC_FEATURE (31)..(31): C-terminal amidation
HSQGTFTSDKSKYLDERAAQDFVQWLLDTKX

SEQ ID NO: 33 - protein, 31 aa, Artificial Sequence
  Synthetic peptide
  MOD_RES (2)..(2): Xaa is AIB
  MISC_FEATURE (10)..(10): Acylated with a C16 fatty acyl group via gamma- Glu-gamma-Glu spacer
  MOD_RES (16)..(16): Xaa is AIB
  MOD_RES (27)..(27): Xaa is Methionine sulfoxide
HXQGTFTSDKSKYLDXRAAQDFVQWLXDTKQ

SEQ ID NO: 34 - protein, 31 aa, Artificial Sequence
  Synthetic peptide
  MOD_RES (2)..(2): Xaa is AIB
  MISC_FEATURE (10)..(10): Acylated with a C16 fatty acyl group via gamma- Glu-gamma-Glu spacer
  MOD_RES (16)..(16): Xaa is AIB
HXQGTFTSDKSKYLDXRAAQDFVQWLLDTKQ

SEQ ID NO: 35 - protein, 31 aa, Artificial Sequence
  Synthetic peptide
  MOD_RES (2)..(2): Xaa is AIB
  MISC_FEATURE (10)..(10): Acylated with a C16 fatty acyl group via gamma- Glu-gamma-Glu spacer
  MOD_RES (16)..(16): Xaa is AIB
  MOD_RES (31)..(31): Xaa is gamma-Glu
  MISC_FEATURE (31)..(31): C-terminal amidation
HXQGTFTSDKSKYLDXRAAQDFVQWLMNTKX

SEQ ID NO: 36 - protein, 11 aa, Artificial Sequence
  Synthetic peptide fragment representing the carboxy terminal 10 amino acids Exendin-4
  MISC_FEATURE (1)..(1): Xaa at position 1 is Lys or Cys
  MISC_FEATURE (11)..(11): Xaa at position 11 is Lys or Cys
XPSSGAPPPSX

SEQ ID NO: 37 - protein, 29 aa, Artificial Sequence
  Synthetic peptide
  MOD_RES (1)..(1): Xaa at position 1 is His, D-His, N-methyl-His, alpha-methyl-His, imidazole acetic acid, des-amino-His, hydroxyl- His, acetyl-His, homo-His, or alpha, alpha-dimethyl imidiazole acetic acid (DMIA)
  MOD_RES (2)..(2): Xaa at position 2 is Ser, D-Ser, Ala, D-Ala, Gly, N-methyl-Ser, AIB, Val, or alpha-amino-N-butyric acid
  MOD_RES (15)..(15): Xaa at position 15 is Asp, Glu, cysteic acid, homoglutamic acid or homocysteic acid;
  MOD_RES (16)..(16): Xaa at position 16 is Glu, Gly, Lys or Aib
  MOD_RES (20)..(20): Xaa at position 20 is Glu, Ala, Lys or Aib
  MOD_RES (21)..(21): Xaa at position 21 is Glu or Aib
  MOD_RES (24)..(24): Xaa at position 24 is Ala, Glu, Lys or Aib
  MOD_RES (27)..(27): Xaa at position 27 is Met, Leu, Val or Nle
XXEGTFTSDVSSYLXXQAAXXFIXWLXKG

SEQ ID NO: 38 - protein, 29 aa, Artificial Sequence
  Synthetic Glucagon Analogue
HAEGTFTSDVSSYLEGQAAKEFIAWLVKG

SEQ ID NO: 39 - protein, 29 aa, Artificial Sequence
  Synthetic Glucagon Analogue
  MOD_RES (16)..(16): Xaa at position 16 is AIB
HAEGTFTSDVSSYLEXQAAKEFIAWLVKG

SEQ ID NO: 40 - protein, 40 aa, Artificial Sequence
  Synthetic Glucagon Analogue
  MISC_FEATURE (40)..(40): Xaa at position 40 is Lys
HAEGTFTSDVSSYLEGQAAKEFIAWLVKGGPSSGAPPPSX

SEQ ID NO: 41 - protein, 40 aa, Artificial Sequence
  Synthetic Glucagon Analogue
  MOD_RES (16)..(16): Xaa at position 16 is AIB
  MOD_RES (40)..(40): Xaa at position 40 is Lys
HAEGTFTSDVSSYLEXQAAKEFIAWLVKGGPSSGAPPPSX

SEQ ID NO: 42 - protein, 11 aa, Artificial Sequence
  Synthetic peptide fragment representing the carboxy terminal 10 amino acids Exendin-4
GPSSGAPPPSK

SEQ ID NO: 43 - protein, 29 aa, Artificial Sequence
  Synthetic peptide
  MOD_RES (1)..(1): Xaa at position 1 is His, D-His, N-methyl-His, alpha-methyl-His, imidazole acetic acid, des-amino-His, hydroxyl- His, acetyl-His, homo-His, or alpha, alpha-dimethyl imidiazole acetic acid (DMIA)
  MOD_RES (2)..(2): Xaa at position 2 is Ser, D-Ser, Ala, D-Ala, Gly, N-methyl-Ser, AIB, Val, or alpha-amino-N-butyric acid
  MOD_RES (3)..(3): Xaa at position 3 is glutamic acid, ornithine, norleucine
  MOD_RES (12)..(12): Xaa at position 12 is Lys or Arg;
  MOD_RES (15)..(15): Xaa at position 15 is Asp, Glu, cysteic acid, homoglutamic acid or homocysteic acid;
  MOD_RES (16)..(16): Xaa at position 16 is Glu, Gln, Gly, Aib, homoglutamic acid, homocysteic acid, or Thr
  MOD_RES (20)..(20): Xaa at position 20 is Glu, Lys or Aib
  MOD_RES (21)..(21): Xaa at position 21 is Glu or Aib
  MOD_RES (24)..(24): Xaa at position 24 is Ala, Glu, Lys or Aib
  MOD_RES (27)..(27): Xaa at position 27 is Met, Leu, Val or Nle
  MOD_RES (28)..(28): Xaa at position 28 is Glu, Lys or AIB
  MOD_RES (29)..(29): Xaa at position 29 is Gly, Gln, Asp or Glu
XXXGTFTSDVSXYLXXQAAXXFIXWLXXX

SEQ ID NO: 44 - protein, 31 aa, Artificial Sequence
  Synthetic Glucagon Analogue
HAEGTFTSDVSSYLEGQAAKEFIAWLVKGRG

SEQ ID NO: 45 - protein, 29 aa, Artificial Sequence
  Synthetic Glucagon Analogue
  MISC_FEATURE (2)..(2): Xaa at position 2 is D-serine or AIB
  MISC_FEATURE (16)..(16): Xaa at position 16 is Gly, Glu or AIB
HXEGTFTSDVSSYLEXQAAKEFIAWLVKG

SEQ ID NO: 46 - protein, 40 aa, Artificial Sequence
  Synthetic Glucagon Analogue
HAEGTFTSDVSSYLEGQAAKEFIAWLVKGGPSSGAPPPSK

SEQ ID NO: 47 - protein, 40 aa, Artificial Sequence
  Synthetic Glucagon Analogue
  MOD_RES (16)..(16): Xaa at position 16 is AIB
HAEGTFTSDVSSYLEXQAAKEFIAWLVKGGPSSGAPPPSK

SEQ ID NO: 48 - protein, 40 aa, Artificial Sequence
  Synthetic Glucagon Analogue
  MISC_FEATURE (2)..(2): Xaa at position 2 is D-serine or AIB
  MISC_FEATURE (16)..(16): Xaa at position 16 is Gly, Glu or AIB
HXEGTFTSDVSSYLEXQAAKEFIAWLVKGGPSSGAPPPSK

SEQ ID NO: 49 - protein, 40 aa, Artificial Sequence
  Synthetic Glucagon Analogue
  MISC_FEATURE (2)..(2): Xaa at position 2 is AIB
HXEGTFTSDVSSYLEEQAAKEFIAWLVKGGPSSGAPPPSK

SEQ ID NO: 50 - protein, 29 aa, Artificial Sequence
  Synthetic Glucagon Analogue
  MISC_FEATURE (2)..(2): Xaa at position 2 is Ser, D-serine or AIB
  MISC_FEATURE (16)..(16): Xaa at position 16 is AIB
HXEGTFTSDVSSYLEXQAAKEFIAWLVKG

(19) United States
(12) Patent Application Publication
(10) Pub. No.: US 2019/0290772 A1
(43) Pub. Date: Sep. 26, 2019

(54) GLUCAGON-LIKE PEPTIDE-1-T3 CONJUGATES

(71) Applicant: INDIANA UNIVERSITY RESEARCH AND TECHNOLOGY CORPORATION, Indianapolis, IN (US)

(72) Inventors: Richard D. DIMARCHI, Carmel, IN (US); Brian FINAN, Indianapolis, IN (US); Bin YANG, Plainfield, IN (US); Zhimeng ZHU, Wuhan, Hubei (CN)

(21) Appl. No.: 16/302,846

(22) PCT Filed: May 26, 2017
(86) PCT No.: PCT/US17/34618

§ 371 Date: Nov. 19, 2018

Related U.S. Application Data
(60) Provisional application No. 62/344,666, filed on Jun. 2, 2016.

(51) Int. Cl. A61K 47/55; A61K 47/54; A61K 47/65; A61K 47/64; A61K 31/197; A61K 38/26

(52) U.S. Cl. CPC A61K 47/55; A61K 47/542; A61K 38/26; A61K 47/64; A61K 31/197; A61K 47/65

(57) ABSTRACT

Provided herein are GLP-1 agonist peptides conjugated with thyroid hormone receptor ligands that are capable of acting at the thyroid hormone receptor. Also provided herein are pharmaceutical compositions and kits of the conjugates of the invention. Further provided herein are methods of treating a disease, e.g., a metabolic disorder, such as diabetes, obesity, metabolic syndrome and chronic cardiovascular disease, comprising administering the conjugates of the invention.

### CROSS REFERENCE TO RELATED APPLICATIONS

This application claims priority to U.S. Provisional Patent Application No. 62/344,666 filed on Jun. 2, 2016, the disclosure of which is hereby expressly incorporated by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 32 kilobytes acii (text) file named “265653seqlist_ST25.txt,” created on May 18, 2017.

### BACKGROUND

When blood glucose begins to fall, glucagon, a hormone produced by the pancreas, signals the liver to break down glycogen and release glucose, causing blood glucose levels to rise toward a normal level. Glucagon-like peptide-1 (GLP-1) has different biological activities compared to glucagon. Its actions include stimulation of insulin synthesis and secretion, inhibition of glucagon secretion, and inhibition of food intake. GLP-1 has been shown to reduce hyperglycemia (elevated glucose levels) in diabetics. Exendin-4, a peptide from lizard venom that shares about 50% amino acid identity with GLP-1, activates the GLP-1 receptor and likewise has been shown to reduce hyperglycemia in diabetics.

There is also evidence that GLP-1 and exendin-4 may reduce food intake and promote weight loss, an effect that would be beneficial not only for diabetics but also for patients suffering from obesity. Patients with obesity have a higher risk of diabetes, hypertension, hyperlipidemia, cardiovascular disease, and musculoskeletal diseases.

Thyroid hormones powerfully influence systemic metabolism through multiple pathways, with profound effects on energy expenditure, fat oxidation, and cholesterol metabolism. Clinical reports revealed sixty years ago that administration of thyroid extracts reduced circulating cholesterol and reversed obesity. However, adverse side effects of thyroid hormone treatment include increased heart rate, cardiac hypertrophy, muscle wasting, and reduced bone density, terminating its clinical use.

Discovery of thyromimetics capable of separating lipid metabolism benefits from adverse cardiovascular effects has remained a desire for patients, physicians and the pharmaceutical industry. Human genomic data and studies in isoform-specific knockout mice have suggested that thyroid hormone receptor alpha (TRα) mediates the hypertrophic cardiovascular actions of thyroid hormones while thyroid hormone receptor beta (TRβ) promotes hepatic lipid metabolism, and both isoforms mediate lipolysis and thermogenesis in adipose tissues. This knowledge has initiated attempts to rationally design small molecules with selective preference for TRβ compared to TRα for the purpose to treat dyslipidemia. Second generation thyromimetics sought isoform specificity and tissue-specific function by derivatization with chemical moieties to promote tissue selectivity.

In accordance with the current disclosure, compositions are provided wherein the hypothalamus/pancreatic-mediated hyperglycemia reducing properties of GLP-1, as well as the appetite suppressant properties are combined with thyroid hormone activity in a single complex. The GLP-1-mediated delivery spares the cardiovascular system from adverse T3 actions. The therapeutic utility of GLP-1 and thyroid hormone pairing provides a new approach in treatment of obesity, type 2 diabetes, and cardiovascular disease.

### SUMMARY

Applicants disclose compositions and methods for GLP-1-mediated selective delivery of thyroid hormone action to the hypothalamus and pancreas as primary targets. Together, coordinated GLP-1 and thyroid hormone actions synergize to correct hyperglycemia and lower body weight. Importantly, the GLP-1-mediated delivery spares adverse action of thyroid hormone on the cardiovascular system.

Provided herein are chemical conjugates of a GLP-1 agonist peptide and compounds having thyroid hormone activity (“GLP-1/T3 conjugates”). These conjugates with plural activities are useful for the treatment of a variety of diseases including hyperglycemia, diabetes and obesity. Advantageously, the disclosed conjugates lack the adverse effects on the cardiovascular system that are associated with T3 administration and also lack the adverse effect of hypoglycemia that can be associated with the administration of GLP-1. The GLP-1/T3 conjugates of the present disclosure can be represented by the following formula:

Q-L-Y

wherein Q is a GLP-1 agonist peptide, Y is a thyroid hormone receptor ligand, and L is a linking group or a bond. In accordance with one embodiment Q is a GLP-1 agonist peptide that exhibits agonist activity at the GLP-1 receptor. In some embodiments, the GLP-1 agonist peptide is a fusion peptide wherein a second peptide has been fused to the C-terminus of the GLP-1 peptide. The thyroid hormone receptor ligand, (Y) is wholly or partly non-peptidic and acts at the thyroid receptor. In some embodiments Y is a compound having the general structure

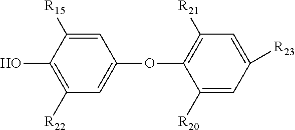

wherein

R₁₅ is C₁-C₄ alkyl, —CH₂(C₆ heteroaryl), —CH₂(OH)(C₆ aryl)F, —CH(OH)CH₃, halo or H;

R₂₀ is halo, CH₃ or H;

R₂₁ is halo, CH₃ or H;

R₂₂ is H, OH, halo, —CH₂(OH)(C₆ aryl)F, and C₁-C₄ alkyl; and

R₂₃ is —CH₂CH(NH₂)COOH, —OCH₂COOH, —NHC(O)COOH, —CH₂COOH,

—NHC(O)CH₂COOH, —CH₂CH₂COOH, or —OCH₂PO₃²⁻. In one embodiment Y is selected from the group consisting of thyroxine T4 (3,5,3′,5′-tetra-iodothyronine), and 3,5,3′-triiodo L-thyronine.

In one embodiment the GLP-1 agonist peptide (Q) component of the complex comprises the sequence

(SEQ ID NO: 43)
X₁X₂X₃GTFTSDVSX₁₂YLX₁₅X₁₆QAAX₂₀X₂₁FIX₂₄WLX₂₇X₂₈X₂₉

wherein

X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);

X₂ is selected from the group consisting of Ser, D-Ser, Ala, D-Ala, Gly, N-methyl-Ser, Aib, Val, or α-amino-N-butyric acid;

X₃ is glutamic acid, ornithine, norleucine;

X₁₂ is Lys or Arg;

X₁₅ is Asp, Glu, cysteic acid, homoglutamic acid or homocysteic acid;

X₁₆ is Aib, Glu, Gln, homoglutamic acid, homocysteic acid, Thr or Gly;

X₂₀ is Glu, Lys or Aib;

X₂₁ is Glu or Aib;

X₂₄ is Ala, Glu, Lys or Aib;

X₂₇ is Met, Val, Leu or Nle;

X₂₈ is Glu, Lys or Aib;

X₂₉ is Gly, Gln, Asp or Glu; optionally comprising an intramolecular bridge between the side chains of amino acids at positions i and i+4, wherein i is 12, 16, 20 or 24, optionally comprising a C-terminal extension of GPSSGAPPPS (SEQ ID NO: 29) linked to position 29.

In some aspects of the invention, pharmaceutical compositions comprising the Q-L-Y conjugate and a pharmaceutically acceptable carrier are also provided.

In other aspects of the present disclosure, methods are provided for administering a therapeutically effective amount of a Q-L-Y conjugate described herein for treating a disease or medical condition in a patient. In some embodiments, the disease or medical condition is selected from the group consisting of metabolic syndrome, diabetes and obesity.

In one embodiment the therapeutic index of the GLP-1-T3 conjugates is enhanced by linking a self-cleaving dipeptide to the active site of the GLP-1 agonist peptide or the thyroid hormone receptor ligand component of the conjugate. Subsequent removal of the dipeptide under physiological conditions and in the absence of enzymatic activity restores full activity to the Q-L-Y conjugate. Advantageously, the dipeptide will chemically cleave (in the absence of enzymatic activity) under physiological conditions at a rate determined in part by the substituents on the dipeptide. In one embodiment the conjugate Q-L-Y is modified by the covalent linkage of one or more dipeptides (A-B) to an amine of Q or Y, wherein A is an amino acid or a hydroxy acid and B is an N-alkylated amino acid linked to Q or Y through an amide bond between a carboxyl moiety of B and an amine of Q and/or Y. In one embodiment both Q and Y are linked to a dipeptide A-B. In one embodiment, A-B comprises the structure:

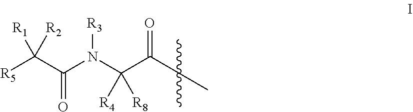

wherein
(a) R¹, R², R⁴ and R⁸ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₁ alkyl)CONH₂, (C₁-C₁ alkyl)COOH, (C₁-C₁ alkyl)NH₂, (C₁-C₄ alkyl)NHC(NH₂⁺)NH₂, (C₀-C₄ alkyl)(C₃-C₆ cycloalkyl), (C₀-C₁ alkyl)(C₂-C₅ heterocyclic), (C₀-C₁ alkyl)(C₆-C₁₀ aryl)R⁷, (C₁-C₄ alkyl)(C₃-C₉ heteroaryl), and C₁-C₁₂ alkyl(W1)C₁-C₁₂ alkyl, wherein W1 is a heteroatom selected from the group consisting of N, S and O, or
(ii) R¹ and R² together with the atoms to which they are attached form a C₃-C₁₂ cycloalkyl or aryl; or
(iii) R⁴ and R⁸ together with the atoms to which they are attached form a C₃-C₆ cycloalkyl;

(b) R³ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R⁷, and (C₁-C₄ alkyl)(C₃-C₉ heteroaryl) or R⁴ and R³ together with the atoms to which they are attached form a 4, 5 or 6 member heterocyclic ring;

(c) R⁵ is NHR⁶ or OH;

(d) R⁶ is H, C₁-C₈ alkyl; and

(e) R⁷ is selected from the group consisting of H and OH

wherein the chemical cleavage half-life (t_{1/2}) of A-B from Q and/or Y is at least about 1 hour to about 1 week in PBS under physiological conditions.

### BRIEF DESCRIPTION OF THE DRAWINGS

FIG. 1 Presents the metabolic pathways for Thyroxine (T4).

FIG. 2 Presents the chemical structures of Triiodothyronine (T3) and various known analogs thereof.

FIG. 3 Presents the chemical structures of L-thyroxine and its enantiomer Dextrothyroxine which was used in an early clinical trial to treat dyslipidemia; as well as the chemical structures of various thyroxine analogs including the organ-selective analogs L-94901 and T-0681, and TRβ1-selective analogs GC-1, CGS23425, KB-141, DITPA, and MB07344, the active form of the prodrug MB07811.

FIG. 4 Presents the chemical structures of Triiodothyronine (T3) and various known analogs thereof.

FIGS. 5A-5D are graphs demonstrating that a GLP-1-Thyroid Hormone conjugate synergistically improves body weight in diet-induced obesity (DIO) mice. The conjugate comprises a GLP-1 analog (IUB48) having Aib at position 2, Glu at position 16 and a C-terminal extension of GPSSGAPPPSK (SEQ ID NO: 42), wherein T3 is linked via the lysine side chain at position 40 of the GLP-1 analog. The conjugate was administered once a day subcutaneously at 25 nmol/kg. The percent decrease in body weight is measured after administration and the administered conjugate has dramatically improved effects relative to the effect seen when the individual compounds (a GLP-1 agonist (IUB48), or triiodothyronine, T3) are administered. At an equimolar dose of 100 nmoles/kg, an absolute decrease in body weight of 15% and 11% from baseline was observed after a week of daily treatment with IUB686 (GLP-1/T3) and IUB687 (GLP-1/iT3), respectively (FIG. 5A). The loss of body weight caused by both conjugates was due to a loss of fat mass (FIG. 5B). Food intake was increased by systemic T3 treatment, recapitulating the hyperphagia associated with central hyperthyroidism in rodents (FIG. 5C). Despite the 11% decrease in body weight following treatment with IUB687, no reductions in food intake were observed with this compound (FIG. 5C). However, treatment IUB686 resulted in a dose-dependent reduction in food intake (FIG. 5C). In respect to conjugate therapies, only treatment with the higher dose of IUB686 resulted in an improved glucose tolerance (FIG. 5D), which is significantly greater than the improved glycemic control achieved by equimolar systemic T3.

FIGS. 6A-6I are graphs demonstrating the effect of a GLP-1—Thyroid Hormone conjugate on blood glucose levels in diet-induced obesity (DIO) mice. Compared to those mice pair-fed to receive the same amount of food as those mice that received IUB686 (FIG. 6A), we see that IUB686 treatment results in greater body weight lowering than the pair-fed controls (FIG. 6B). Both T3 alone and IUB686 (GLP-1/T3) substantially increased whole-body energy expenditure but to differing degrees with IUB686 causing significantly less energy expenditure than systemic T3 (FIG. 6C). With systemic T3 alone, the observed hyperphagia (FIG. 6D) partially compensated for the increased energy expenditure, which is in magnitude is greater than achieved by IUB686 therapy. This resulted in in less body weight loss delivered by T3 compared to that achieved by IUB686 therapy, which itself drove a negative energy balance resulting in a loss of body weight (FIG. 6E). Furthermore, systemic T3 significantly increased home cage activity (FIG. 6F), which paralleled the observed increase in energy expenditure. Unlike mono-therapy with T3 however, the conjugate did not cause an increase in ambulatory activity (FIG. 6F), demonstrating that altered activity is not contributing to the enhanced energy expenditure caused by the GLP-1 based conjugate. Additionally, IUB686 caused a decrease in the respiratory exchange ratio (RER) (FIG. 6G) to a greater extent than what is achieved by GLP-1 monotherapy (IUB48). The magnitude of the cholesterol lowering effect is also greater than that achieved by systemic T3 alone (FIG. 6H). However, unlike systemic T3 therapy, which induced cardiac hypertrophy, IUB686 did not increase raw heart weight (FIG. 6I).

FIGS. 7A-7H are graphs demonstrating the contribution of central GLP-1R signaling to the metabolic benefits of IUB686. The effect of IUB686 to lower body weight (FIG. 7A) and improve glucose tolerance (FIG. 7C) in global GLP-1R−/− mice made obese by HFHSD feeding was investigated. In these global GLP-1R−/− mice, the effects of IUB686 were completely lost relative to wild-type mice (FIGS. 7B & 7D). When the GLP-1/T3 conjugate was administered to mice having the genetic deletion of GLP-1R in the central nervous system (CNS) and maintained on a HFHSD, the effects of IUB686 to lower body weight were substantially blunted (compare FIG. 7E, wild type mice to FIG. 7F, GLP-1R−/− mice). The improved glucose tolerance delivered by chronic IUB686 treatment seen in wild-type mice is preserved in CNS-specific GLP-1R−/− mice (compare FIG. 7G to FIG. 7H)

### DETAILED DESCRIPTION

### Definitions

In describing and claiming the invention, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein the term “amino acid” encompasses any molecule containing both amino and carboxyl functional groups, wherein the amino and carboxylate groups are attached to the same carbon (the alpha carbon). The alpha carbon optionally may have one or two further organic substituents. For the purposes of the present disclosure designation of an amino acid without specifying its stereochemistry is intended to encompass either the L or D form of the amino acid, or a racemic mixture.

As used herein the term “non-coded amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

A “bioactive polypeptide” refers to polypeptides which are capable of exerting a biological effect in vitro and/or in vivo.

As used herein a general reference to a peptide is intended to encompass peptides that have modified amino and carboxy termini. For example, an amino acid sequence designating the standard amino acids is intended to encompass standard amino acids at the N- and C-terminus as well as a corresponding hydroxyl acid at the N-terminus and/or a corresponding C-terminal amino acid modified to comprise an amide group in place of the terminal carboxylic acid.

As used herein an “acylated” amino acid is an amino acid comprising an acyl group which is non-native to a naturally-occurring amino acid, regardless by the means by which it is produced. Exemplary methods of producing acylated amino acids and acylated peptides are known in the art and include acylating an amino acid before inclusion in the peptide or peptide synthesis followed by chemical acylation of the peptide. In some embodiments, the acyl group causes the peptide to have one or more of (i) a prolonged half-life in circulation, (ii) a delayed onset of action, (iii) an extended duration of action, (iv) an improved resistance to proteases, and (v) increased potency at the IGF and/or insulin peptide receptors.

As used herein, an “alkylated” amino acid is an amino acid comprising an alkyl group which is non-native to a naturally-occurring amino acid, regardless of the means by which it is produced. Exemplary methods of producing alkylated amino acids and alkylated peptides are known in the art and including alkylating an amino acid before inclusion in the peptide or peptide synthesis followed by chemical alkylation of the peptide. Without being held to any particular theory, it is believed that alkylation of peptides will achieve similar, if not the same, effects as acylation of the peptides, e.g., a prolonged half-life in circulation, a delayed onset of action, an extended duration of action, an improved resistance to proteases and increased potency at the IGF and/or insulin receptors.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” refers to salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, the term “hydrophilic moiety” refers to any compound that is readily water-soluble or readily absorbs water, and which are tolerated in vivo by mammalian species without toxic effects (i.e. are biocompatible). Examples of hydrophilic moieties include polyethylene glycol (PEG), polylactic acid, polyglycolic acid, a polylactic-polyglycolic acid copolymer, polyvinyl alcohol, polyvinylpyrrolidone, polymethoxazoline, polyethyloxazoline, polyhydroxyethyl methacrylate, polyhydroxypropyl methacrylamide, polymethacrylamide, polydimethylacrylamide, and derivatised celluloses such as hydroxymethylcellulose or hydroxyethylcellulose and co-polymers thereof, as well as natural polymers including, for example, albumin, heparin and dextran.

As used herein, the term “treating” includes prophylaxis of the specific disorder or condition, or alleviation of the symptoms associated with a specific disorder or condition and/or preventing or eliminating said symptoms. For example, as used herein the term “treating diabetes” will refer in general to maintaining glucose blood levels near normal levels and may include increasing or decreasing blood glucose levels depending on a given situation.

As used herein an “effective” amount or a “therapeutically effective amount” of a bioactive agent (e.g., glucaon or GLP-1 analog) refers to a nontoxic but sufficient amount of the bioactive agent to provide the desired effect. For example one desired effect would be the prevention or treatment of hyperglycemia. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as intranasal, inhalation, subcutaneous, intramuscular, intraspinal, or intravenous.

As used herein the term “derivative” is intended to encompass chemical modification to a compound (e.g., an amino acid), including chemical modification in vitro, e.g. by introducing a group in a side chain in one or more positions of a polypeptide, e.g. a nitro group in a tyrosine residue, or iodine in a tyrosine residue, or by conversion of a free carboxylic group to an ester group or to an amide group, or by converting an amino group to an amide by acylation, or by acylating a hydroxy group rendering an ester, or by alkylation of a primary amine rendering a secondary amine or linkage of a hydrophilic moiety to an amino acid side chain. Other derivatives are obtained by oxidation or reduction of the side-chains of the amino acid residues in the polypeptide.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As used herein, the term “selectivity” of a molecule for a first receptor relative to a second receptor refers to the following ratio: EC₅₀ of the molecule at the second receptor divided by the EC₅₀ of the molecule at the first receptor. For example, a molecule that has an EC₅₀ of 1 nM at a first receptor and an EC₅₀ of 100 nM at a second receptor has 100-fold selectivity for the first receptor relative to the second receptor.

The term “GLP-1 agonist peptide” refers to a compound that binds to and activates downstream signaling of the GLP-1 receptor. The GLP-1 agonist peptide may also have agonist activity at other receptors such as the GIP receptor.

As used herein, “thyroid hormone receptor ligand” refers to a compound that has biological agonist activity and binds to and activates downstream signaling of the thyroid hormone receptor. The thyroid hormone receptor ligand is wholly or partly non-peptidic.

As used herein an amino acid “modification” refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids may be purchased from commercial suppliers, synthesized de novo, or chemically modified or derivatized from naturally occurring amino acids.

As used herein an amino acid “substitution” refers to the replacement of one amino acid residue by a different amino acid residue.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln, cysteic acid and homocysteic acid;

III. Polar, positively charged residues:
His, Arg, Lys; Ornithine (Orn)

IV. Large, aliphatic, nonpolar residues:
Met, Leu, Ile, Val, Cys, Norleucine (Nle), homocysteine

V. Large, aromatic residues:
Phe, Tyr, Trp, acetyl phenylalanine

Throughout the application, all references to a particular amino acid position by number (e.g., position 28) refer to the amino acid at that position in the respective native amino acid sequence (e.g. native glucagon, native GLP-1). For example, a reference herein to “position 28” would mean the corresponding position 27 for an analog of SEQ ID NO: 1 in which the first amino acid of SEQ ID NO: 1 has been deleted. Similarly, a reference herein to “position 28” would mean the corresponding position 29 for an analog of SEQ ID NO: 1 in which one amino acid has been added before the N-terminus of SEQ ID NO: 1. In addition a reference to a position greater than 29 (native glucagon (SEQ ID NO: 1) only has 29 amino acids) is intended to refer to amino acid position in an analog having a C-terminus amino acid extension after the corresponding position 29 of SEQ ID NO: 1.

As used herein the general term “polyethylene glycol chain” or “PEG chain”, refers to mixtures of condensation polymers of ethylene oxide and water, in a branched or straight chain, represented by the general formula H(OCH₂CH₂)—OH, wherein n is at least 2. “Polyethylene glycol chain” or “PEG chain” is used in combination with a numeric suffix to indicate the approximate average molecular weight thereof. For example, PEG-5,000 refers to polyethylene glycol chain having a total molecular weight average of about 5,000 Daltons.

As used herein the term “pegylated” and like terms refers to a compound that has been modified from its native state by linking a polyethylene glycol chain to the compound. A “pegylated polypeptide” is a polypeptide that has a PEG chain covalently bound to the polypeptide.

As used herein a “linker” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties and enzyme-cleavable groups.

The term “C₁-Cₙ alkyl” wherein n can be from 1 through 6, as used herein, represents a branched or linear alkyl group having from one to the specified number of carbon atoms. Typical C₁-C₆ alkyl groups include, but are not limited to, methyl, ethyl, n-propyl, iso-propyl, butyl, iso-Butyl, sec-butyl, tert-butyl, pentyl, hexyl and the like.

The terms “C₂-Cₙ alkenyl” wherein n can be from 2 through 6, as used herein, represents an olefinically unsaturated branched or linear group having from 2 to the specified number of carbon atoms and at least one double bond. Examples of such groups include, but are not limited to, 1-propenyl, 2-propenyl (—CH₂—CH═CH₂), 1,3-butadienyl, (—CH═CHCH═CH₂), 1-butenyl (—CH═CHCH₂CH₃), hexenyl, pentenyl, and the like.

The term “C₂-Cₙ alkynyl” wherein n can be from 2 to 6, refers to an unsaturated branched or linear group having from 2 to n carbon atoms and at least one triple bond. Examples of such groups include, but are not limited to, 1-propynyl, 2-propynyl, 1-butynyl, 2-butynyl, 1-pentynyl, and the like.

As used herein the term “aryl” refers to a mono- or bicyclic carbocyclic ring system having one or two aromatic rings including, but not limited to, phenyl, naphthyl, tetrahydronaphthyl, indanyl, indenyl, and the like. The size of the aryl ring and the presence of substituents or linking groups are indicated by designating the number of carbons present. For example, the term “(C₁-C₃alkyl)(C₆-C₁₀ aryl)” refers to a 5 to 10 membered aryl that is attached to a parent moiety via a one to three membered alkyl chain.

The term “heteroaryl” as used herein refers to a mono- or bi-cyclic ring system containing one or two aromatic rings and containing at least one nitrogen, oxygen, or sulfur atom in an aromatic ring. The size of the heteroaryl ring and the presence of substituents or linking groups are indicated by designating the number of carbons present. For example, the term “(C₁-Cₙ alkyl)(C₅-C₆ heteroaryl)” refers to a 5 or 6 membered heteroaryl that is attached to a parent moiety via a one to “n” membered alkyl chain.

As used herein, the term “halo” refers to one or more members of the group consisting of fluorine, chlorine, bromine, and iodine.

As used herein the term “patient” without further designation is intended to encompass any warm blooded vertebrate domesticated animal (including for example, but not limited to livestock, horses, cats, dogs and other pets) and humans.

The term “isolated” as used herein means having been removed from its natural environment. In some embodiments, the analog is made through recombinant methods and the analog is isolated from the host cell.

The term “purified,” as used herein relates to the isolation of a molecule or compound in a form that is substantially free of contaminants normally associated with the molecule or compound in a native or natural environment and means having been increased in purity as a result of being separated from other components of the original composition. The term “purified polypeptide” is used herein to describe a polypeptide which has been separated from other compounds including, but not limited to nucleic acid molecules, lipids and carbohydrates.

As used herein, the term “peptide” encompasses a sequence of 2 or more amino acids and typically less than 50 amino acids, wherein the amino acids are naturally occurring or coded or non-naturally occurring or non-coded amino acids. Non-naturally occurring amino acids refer to amino acids that do not naturally occur in vivo but which, nevertheless, can be incorporated into the peptide structures described herein. “Non-coded” as used herein includes an amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

As used herein the term “hydroxyl acid” refers to an amino acid that has been modified to replace the alpha carbon amino group with a hydroxyl group.

As used herein, “partly non-peptidic” refers to a molecule wherein a portion of the molecule is a chemical compound or substituent that has biological activity and that does not comprises a sequence of amino acids.

A “peptidomimetic” refers to a chemical compound having a structure that is different from the general structure of an existing peptide, but that functions in a manner similar to the existing peptide, e.g., by mimicking the biological activity of that peptide. Peptidomimetics typically comprise naturally-occurring amino acids and/or unnatural amino acids, but can also comprise modifications to the peptide backbone. For example a peptidomimetic may include a sequence of naturally-occurring amino acids with the insertion or substitution of a non-peptide moiety, e.g. a retroinverso fragment, or incorporation of non-peptide bonds such as an azapeptide bond (CO substituted by NH) or pseudo-peptide bond (e.g. NH substituted with CH₂), or an ester bond (e.g., depsipeptides, wherein one or more of the amide (—CONHR—) bonds are replaced by ester (COOR) bonds). Alternatively the peptidomimetic may be devoid of any naturally-occurring amino acids.

As used herein the term “charged amino acid” or “charged residue” refers to an amino acid that comprises a side chain that is negatively charged (i.e., de-protonated) or positively charged (i.e., protonated) in aqueous solution at physiological pH. For example, negatively charged amino acids include aspartic acid, glutamic acid, cysteic acid, homocysteic acid, and homoglutamic acid, whereas positively charged amino acids include arginine, lysine and histidine. Charged amino acids include the charged amino acids among the 20 amino acids commonly found in human proteins, as well as atypical or non-naturally occurring amino acids.

As used herein the term “acidic amino acid” refers to an amino acid that comprises a second acidic moiety (other than the alpha carboxylic acid of the amino acid), including for example, a side chain carboxylic acid or sulfonic acid group.

As used herein, the term “prodrug” is defined as any compound that undergoes chemical modification before exhibiting its full pharmacological effects.

As used herein, a “dipeptide” is the result of the linkage of an α-amino acid or α-hydroxyl acid to another amino acid, through a peptide bond.

As used herein the term “chemical cleavage” absent any further designation encompasses a non-enzymatic reaction that results in the breakage of a covalent chemical bond.

As used herein the term “GLP-1/T3 conjugates” is a generic reference to any conjugate that comprises a peptide having the ability to bind and activate the GLP-1 receptor and a second compound having the ability to bind and activate the thyroid hormone receptor.

### EMBODIMENTS

Thyroid hormones have profound effects on lipid, cholesterol and glucose metabolism through liver-specific actions. Thyroid hormones also have substantial effects on thermogenesis and lipolysis through adipose-specific actions. These combined actions make thyroid hormone an attractive drug candidate for the treatment of dyslipidemia and obesity. However, adverse effects primarily in the cardiovascular system have until now precluded its use for chronic treatment of metabolic diseases. Importantly, the beneficial functions of thyroid hormone on systemic metabolism are largely aligned with chronic actions of GLP-1 on body weight. As disclosed herein by using GLP-1 as a targeting ligand, unbiased thyroid hormone action can be selectively guided to the hypothalamus and the pancreas, where synergistic benefits on adiposity are unleashed. Importantly, the disclosed conjugates uncouple the metabolic benefits from deleterious effects on the cardiovascular system that would otherwise arise from systemic thyroid hormone action. Furthermore, the effects of thyroid hormone action counteract the diabetogenic effects of GLP-1 action, completing mutual cancellation of the inherent limitations of each hormone. Unimolecular integration of thyroid hormone and GLP-1 action profiles synergize to maximize comprehensive metabolic benefits while masking their harmful effects that had prevented their individual use.

Applicants disclose herein compositions and methods for GLP-1-mediated selective delivery of thyroid hormone action to the hypothalamus and pancreas. Together, coordinated GLP-1 and thyroid hormone actions synergize to lower body weight through liver and fat-specific mechanisms.

Provided herein are chemical conjugates of a GLP-1 agonist peptide and compounds having thyroid hormone activity (“GLP-1/T3 conjugates”). These conjugates with plural activities are useful for the treatment of a variety of diseases including hyperlipidemia, metabolic syndrome, diabetes, and obesity.

As disclosed herein chemical conjugates of GLP-1 and thyroid hormone (GLP-1/T3) have been engineered to capitalize on the preferential sites of GLP-1 action to precisely harness T3 action in select tissues. Coordinated GLP-1 and T3 actions synergize to correct hyperlipidemia, hepatic steatosis, atherosclerosis, glucose intolerance and obesity in patients. Each hormonal constituent of the conjugate retains its native activity and mutually enriches cellular processes in hepatocytes and adipocytes. Synchronized signaling driven by GLP-1 and T3 reciprocally minimizes the inherent harmful effects of each hormone. Liver directed T3 action offsets the diabetogenic liability of GLP-1, and GLP-1-mediated delivery spares the cardiovascular system from adverse T3 action.

The GLP-1 agonist peptide conjugates of the present disclosure can be represented by the following formula:

Q-L-Y

wherein Q is a GLP-1 agonist peptide, Y is a thyroid hormone receptor ligand, and L is a linking group or a bond joining Q to Y.

Compounds that are thyroid hormone receptor ligands, particularly selective agonists of the thyroid hormone receptor, are expected to demonstrate a utility for the treatment or prevention of diseases or disorders associated with thyroid hormone activity, for example: (1) hypercholesterolemia, dyslipidemia or any other lipid disorder manifested by an unbalance of blood or tissue lipid levels; (2) atherosclerosis; (3) replacement therapy in elderly subjects with hypothyroidism who are at risk for cardiovascular complications; (4) replacement therapy in elderly subjects with subclinical hypothyroidism who are at risk for cardiovascular complications; (5) obesity; (6) diabetes (7) depression; (8) osteoporosis (especially in combination with a bone resorption inhibitor); (9) goiter; (10) thyroid cancer; (11) cardiovascular disease or congestive heart failure; (12) glaucoma; and (13) skin disorders.

In accordance with one embodiment a method of inducing weight loss or preventing weight gain is provided, wherein the method comprises administering a GLP-1/T3 conjugate to a patient in need thereof. The weight loss following GLP-1/T3 therapy is due to increased energy expenditure, some of which is mediated via lipolytic mechanisms and the recruitment of thermogenesic-capable adipocytes in iWAT. The primary mechanism responsible for non-shivering thermogenesis in adipocytes is coordinated lipolysis and concurrent uncoupling of the mitochondrial respiratory chain via UCP1 to allow for rapid fatty acid oxidation, minimal ATP production, and heat production. Both GLP-1 and T3 have individually been reported to increase UCP1 activity in vivo.

Importantly, the synergistic effects of GLP-1 and T3 co-agonism translate to less reliance on individual signaling cues to have equal potency as the single hormones. Thus lower circulating concentrations of the conjugate are needed to elicit lipid lowering and body weight-lowering effects, which presumably contributes to the enhanced safety profile.

### Thyroid Hormone Receptor Ligand Agonists

Thyroxine (T₄) is a thyroid hormone involved in the control of cellular metabolism. Chemically, thyroxine is an iodinated derivative of the amino acid tyrosine. The maintenance of a normal level of thyroxine is important for normal growth and development of children as well as for proper bodily function in the adult. Its absence leads to delayed or arrested development. Hypothyroidism, a condition in which the thyroid gland fails to produce enough thyroxine, leads to a decrease in the general metabolism of all cells, most characteristically measured as a decrease in nucleic acid and protein synthesis, and a slowing down of all major metabolic processes. Conversely, hyperthyroidism is an imbalance of metabolism caused by overproduction of thyroxine.

During metabolism, T4 is converted to T3 or to rT3 via removal of an iodine atom from one of the hormonal rings. T3 is the biologically active thyroid hormone, whereas rT3 has no biological activity. Both T3 and T4 are used to treat thyroid hormone deficiency (hypothyroidism). They are both absorbed well by the gut, so can be given orally.

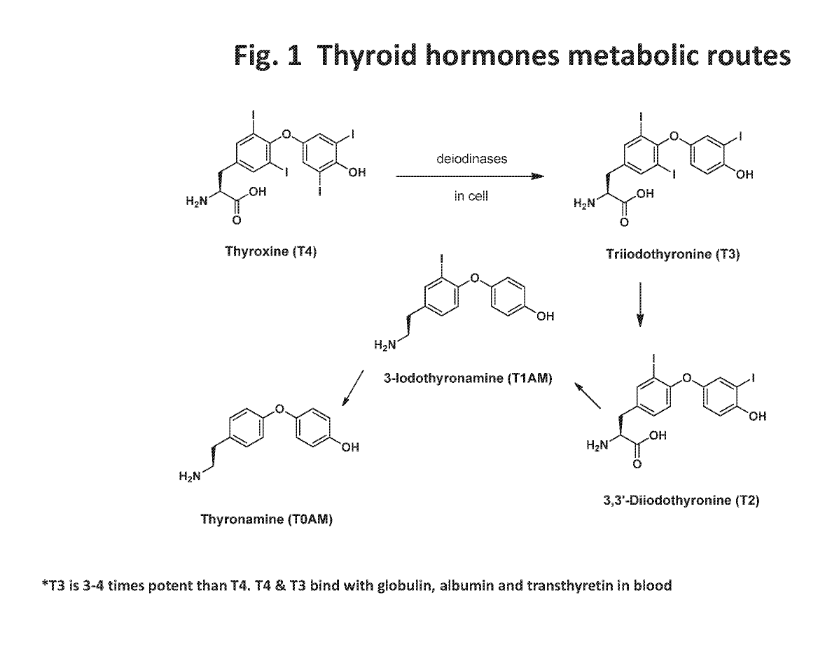

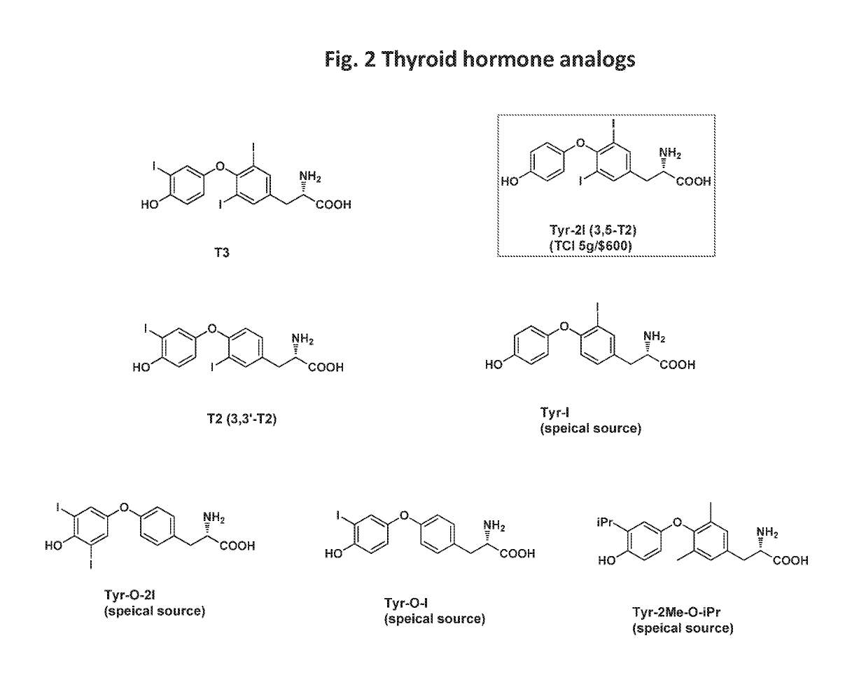

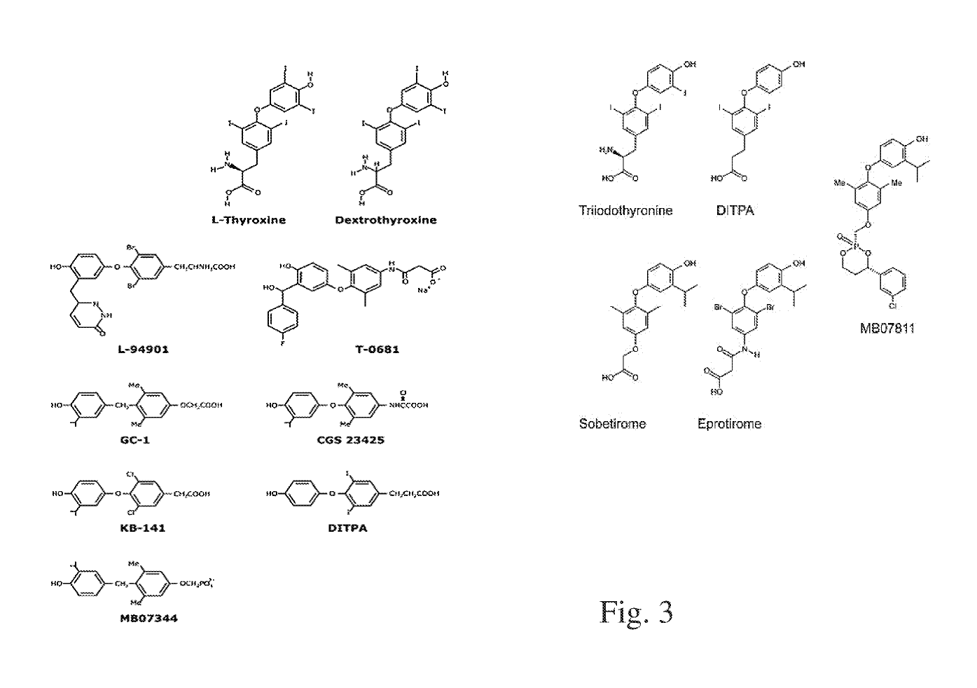

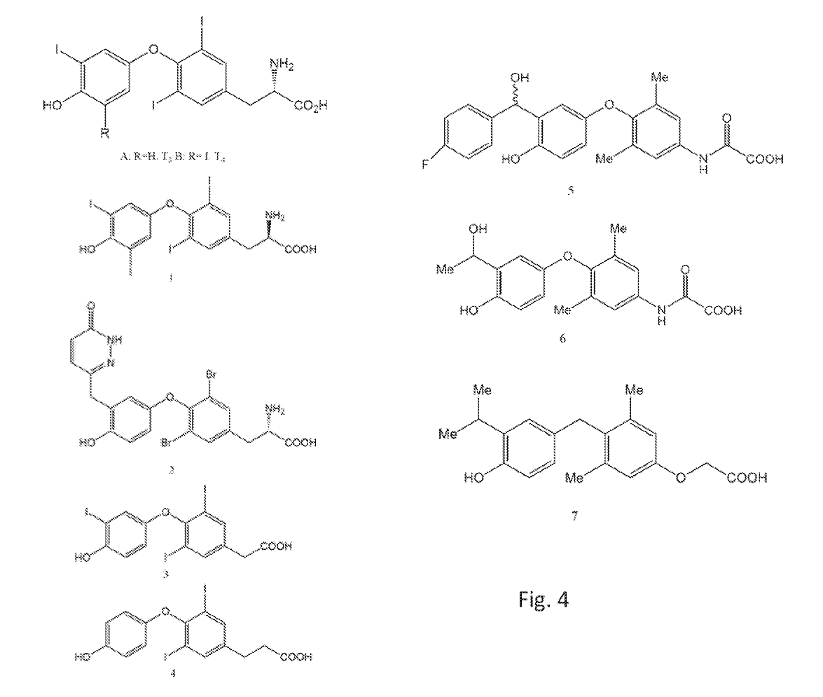

In accordance with the present disclosure a conjugate is provided comprising a thyroid receptor ligand that is covalently linked to a GLP-1 agonist peptide. More particularly in one embodiment the thyroid receptor ligand (Y) of the Q-L-Y conjugate, is thyroid hormone or a thyroid hormone receptor agonist that binds and activates the thyroid receptor. Suitable compounds include any of the compounds disclosed in FIGS. 1-4 or a compound having the general structure of

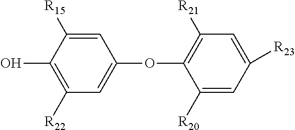

wherein

R₁₅ is C₁-C₄ alkyl, —CH₂(pyridazinone), —CH₂(OH)(phenyl)F, —CH(OH)CH₃, halo or H;

R₂₀ is halo, CH₃ or H;

R₂₁ is halo, CH₃ or H;

R₂₂ is H, OH, halo, —CH₂(OH)(C₆ aryl)F, and C₁-C₄ alkyl; and

R₂₃ is —CH₂CH(NH₂)COOH, —OCH₂COOH, —NHC(O)COOH, —CH₂COOH, —NHC(O)CH₂COOH, —CH₂CH₂COOH, or —OCH₂PO₃²⁻.

In accordance with one embodiment the thyroid hormone component (Y) is a compound of the general structure

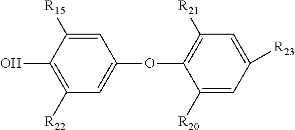

wherein

R₁₅ is C₁-C₄ alkyl, —CH(OH)CH₃, I or H

R₂₀ is I, Br, CH₃ or H;

R₂₁ is I, Br, CH₃ or H;

R₂₂ is H, OH, I, and C₁-C₄ alkyl; and

R₂₃ is —CH₂CH(NH₂)COOH, —OCH₂COOH, —NHC(O)COOH, —CH₂COOH, —NHC(O)CH₂COOH, —CH₂CH₂COOH, or —OCH₂PO₃²⁻. In one embodiment R₂₃ is —CH₂CH(NH₂)COOH.

In accordance with one embodiment the thyroid hormone component (Y) is a compound of the general structure

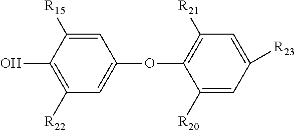

wherein

R₁₅ is isopropyl, —CH(OH)CH₃, I or H

R₂₀ is I, Br, Cl, or CH₃;

R₂₁ is I, Br, Cl, or CH₃;

R₂₂ is H; and

R₂₃ is —OCH₂COOH, —CH₂COOH, —NHC(O)CH₂COOH, or —CH₂CH₂COOH.

In accordance with one embodiment the thyroid hormone component (Y) is a compound of the general structure of Formula I:

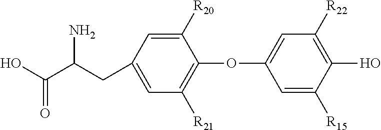

wherein

R₂₀, R₂₁, and R₂₂ are independently selected from the group consisting of H, OH, halo and C₁-C₄ alkyl; and

R₁₅ is halo or H. In one embodiment R₂₀ and R₂₁ are each CH₃, R₁₅ is H and R₂₂ is independently selected from the group consisting of H, OH, halo and C₁-C₄ alkyl. In one embodiment R₂₀, R₂₁ and R₂₂ are each halo and R₁₅ is H or halo. In a further embodiment R₂₀, R₂₁ and R₂₂ are each independently I or Cl, and R₁₅ is H, Cl or I. In a further embodiment a compound of Formula I is provided wherein R₂₀, R₂₁ and R₂₂ are each I, and R₁₅ is H or I.

In accordance with one embodiment Y is selected from the group consisting of thyroxine T4 (3,5,3′,5′-tetraiodothyronine) and 3,5,3′-triiodo L-thyronine.

In one embodiment, the thyroid receptor ligand (Y) of the Q-L-Y conjugates, is an indole derivative of thyroxine, including for example, compounds disclosed in U.S. Pat. No. 6,794,406 and US published application no. US 2009/0233979, the disclosures of which are incorporated herein. In one embodiment the indole derivative of thyroxine comprises a compound of the general structure of Formula II:

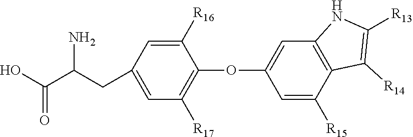

wherein

R₁₃ is H or C₁-C₄ alkyl;

R₁₄ is C₁-C₈ alkyl;

R₁₅ is H or C₁-C₄ alkyl;

R₁₆ and R₁₇ are independently halo or C₁-C₄ alkyl.

In one embodiment, the thyroid receptor ligand (Y) of the Q-L-Y conjugates, is an indole derivative of thyroxine as disclosed in WO97/21993 (U. Cal SF), WO99/00353 (KaroBio), GB98/284425 (KaroBio), and U.S. Provisional Application 60/183,223, the disclosures of which are incorporated by reference herein. In one embodiment the thyroid receptor ligand comprises the general structure of Formula III:

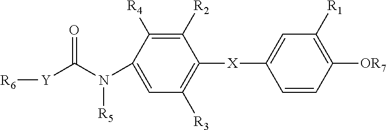

wherein X is oxygen, sulfur, carbonyl, methylene, or NH;

Y is (CH₂)ₙ where n is an integer from 1 to 5, or C═C;

R₁ is halo, trifluoromethyl, or C₁-C₆ alkyl or C₃-C₇ cycloalkyl;

R₂ and R₃ are the same or different and are hydrogen, halogen, C₁-C₆ alkyl or C₃-C₇ cycloalkyl, with the proviso that at least one of R₂ and R₃ is other than hydrogen;

R₄ is hydrogen or C₁-C₄ alkyl;

R₅ is hydrogen or C₁-C₄ alkyl;

R₆ is carboxylic acid, or ester thereof;

R₇ is hydrogen, or an alkanoyl or aroyl group.

### The GLP-1 Agonist Peptide (Q)

In one embodiment, Q of the Q-L-Y conjugates described herein is a native GLP-1 peptide comprising or consisting of the sequence of SEQ ID NO: 44, or a C-terminal truncated analog of native GLP-1 including for example a peptide comprising or consisting of the sequence of SEQ ID NO: 38 and analogs thereof. In one embodiment Q is a GLP-1 analog that retains activity at the GLP-1 receptor and comprise one or more of the modifications disclosed herein or any combination thereof. Examples of modifications that can be made to the native GLP-1 peptide sequence include the following:

Acylation

In accordance with some embodiments, the GLP-1 analog comprises an acylated amino acid (e.g., a non-coded acylated amino acid (e.g., an amino acid comprising an acyl group which is non-native to a naturally-occurring amino acid)). The acylated amino acid in some embodiments causes the GLP-1 analog to have one or more of (i) a prolonged half-life in circulation, (ii) a delayed onset of action, (iii) an extended duration of action, (iv) an improved resistance to proteases, such as DPP-IV, and (v) increased potency at one or both of the GLP-1 and glucagon receptors.

In accordance with one embodiment, the GLP-1 analog comprises an acyl group which is attached to the GLP-1 analog via an ester, thioester, or amide linkage for purposes of prolonging half-life in circulation and/or delaying the onset of and/or extending the duration of action and/or improving resistance to proteases such as DPP-IV.

Acylation can be carried out at any position within the GLP-1 analog, including any of positions 1-31, a position C-terminal to the 29^{th} amino acid (e.g., the amino acid at position 30, 31, 32, 33, 34, 35, 36, 37, 38, 39, 40, 41, 42, 43, 44, 45, 46, 47, etc., at a position within a C-terminal extension or at the C-terminus), provided that GLP-1 activity is retained, if not enhanced. Nonlimiting examples include positions 5, 7, 10, 11, 12, 13, 14, 16, 17, 18, 19, 20, 21, 24, 27, 28, or 29. In exemplary embodiments, the GLP-1 analog comprises an acylated amino acid at one or more positions selected from the group consisting of: 9, 10, 12, 16, and 20. In exemplary embodiments, the GLP-1 analog comprises an acylated amino acid at one or more positions selected from the group consisting of: 10, 12, and 16. In exemplary embodiments, the GLP-1 analog comprises an acylated amino acid at one or more positions selected from the group consisting of: 9, 10, 12, 16, and 20. In exemplary embodiments, the GLP-1 analog comprises an acylated amino acid at one or more positions 10 and 12. In exemplary embodiments, the GLP-1 analog comprises an acylated amino acid at position 12. In exemplary embodiments, the GLP-1 analog comprises a C-terminal extension and an acylated amino acid at one or more positions selected from the group consisting of 9, 10, 12, 16, 20, and 37-43 (e.g., 40). In specific embodiments, acylation occurs at position 10 of the GLP-1 analog and the GLP-1 analog lacks an intramolecular bridge, e.g., a covalent intramolecular bridge (e.g., a lactam bridge). Such acylated GLP-1 analogs lacking an intramolecular bridge demonstrate enhanced activity at the GLP-1 and GLP-1 receptors as compared to the corresponding non-acylated analogs lacking a covalent intramolecular bridge and in comparison to the corresponding analogs lacking an intramolecular bridge acylated at a position other than position 10.

The GLP-1 analog in some embodiments are acylated at the same amino acid position where a hydrophilic moiety is linked, or at a different amino acid position. Nonlimiting examples include acylation at position 10 and pegylation at one or more positions in the C-terminal portion of the GLP-1 analog, e.g., position 24, 28 or 29, within a C-terminal extension, or at the C-terminus (e.g., through adding a C-terminal Cys).

The acyl group can be covalently linked directly to an amino acid of the GLP-1 analog, or indirectly to an amino acid of the GLP-1 analog via a spacer, wherein the spacer is positioned between the amino acid of the GLP-1 analog and the acyl group.

In specific aspects, the GLP-1 analog is modified to comprise an acyl group by direct acylation of an amine, hydroxyl, or thiol of a side chain of an amino acid of the GLP-1 analog. In some embodiments, acylation is at position 10, 20, 24, or 29 of the GLP-1 analog. In this regard, the acylated GLP-1 analog can comprise the amino acid sequence of SEQ ID NO: 38, or a modified amino acid sequence thereof comprising one or more of the amino acid modifications described herein, with at least one of the amino acids at positions 10, 20, 24, and 29 of the analog modified to any amino acid comprising a side chain amine, hydroxyl, or thiol. In some specific embodiments, the direct acylation of the GLP-1 analog occurs through the side chain amine, hydroxyl, or thiol of the amino acid at position 10.

In some embodiments, the amino acid comprising a side chain amine is an amino acid of Formula I:

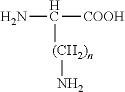

wherein n=1 to 4

[Formula I]

In some exemplary embodiments, the amino acid of Formula I, is the amino acid wherein n is 4 (Lys) or n is 3 (Orn).

In other embodiments, the amino acid comprising a side chain hydroxyl is an amino acid of Formula II:

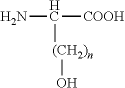

wherein n=1 to 4

[Formula II]

In some exemplary embodiments, the amino acid of Formula II is the amino acid wherein n is 1 (Ser).

In yet other embodiments, the amino acid comprising a side chain thiol is an amino acid of Formula III:

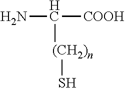

wherein n=1 to 4

[Formula III]

In some exemplary embodiments, the amino acid of Formula III is the amino acid wherein n is 1 (Cys).

In yet other embodiments, the amino acid comprising a side chain amine, hydroxyl, or thiol is a disubstituted amino acid comprising the same structure of Formula I, Formula II, or Formula III, except that the hydrogen bonded to the alpha carbon of the amino acid of Formula I, Formula II, or Formula III is replaced with a second side chain.

In some embodiments, the acylated GLP-1 comprises a spacer between the analog and the acyl group. In some embodiments, the GLP-1 analog is covalently bound to the spacer, which is covalently bound to the acyl group.

In some embodiments, the spacer is an amino acid comprising a side chain amine, hydroxyl, or thiol, or a dipeptide or tripeptide comprising an amino acid comprising a side chain amine, hydroxyl, or thiol. The amino acid to which the spacer is attached can be any amino acid (e.g., a singly or doubly α-substituted amino acid) comprising a moiety which permits linkage to the spacer. For example, an amino acid comprising a side chain NH₂, —OH, or —COOH (e.g., Lys, Orn, Ser, Asp, or Glu) is suitable. In this respect, the acylated GLP-1 analog can comprise the amino acid sequence of SEQ ID NO: 38, or a modified amino acid sequence thereof comprising one or more of the amino acid modifications described herein, with at least one of the amino acids at positions 10, 20, 24, and 29 modified to any amino acid comprising a side chain amine, hydroxyl, or carboxylate.

In some embodiments, the spacer is an amino acid comprising a side chain amine, hydroxyl, or thiol, or a dipeptide or tripeptide comprising an amino acid comprising a side chain amine, hydroxyl, or thiol.

When acylation occurs through an amine group of a spacer, the acylation can occur through the alpha amine of the amino acid or a side chain amine. In the instance in which the alpha amine is acylated, the amino acid of the spacer can be any amino acid. For example, the amino acid of the spacer can be a hydrophobic amino acid, e.g., Gly, Ala, Val, Leu, Ile, Trp, Met, Phe, Tyr, 6-amino hexanoic acid, 5-aminovaleric acid, 7-aminoheptanoic acid, and 8-aminooctanoic acid. Alternatively, the amino acid of the spacer can be an acidic residue, e.g., Asp, Glu, homoglutamic acid, homocysteic acid, cysteic acid, gamma-glutamic acid.

In the instance in which the side chain amine of the amino acid of the spacer is acylated, the amino acid of the spacer is an amino acid comprising a side chain amine, e.g., an amino acid of Formula I (e.g., Lys or Orn). In this instance, it is possible for both the alpha amine and the side chain amine of the amino acid of the spacer to be acylated, such that the GLP-1 analog is diacylated. Embodiments of the invention include such diacylated molecules.

When acylation occurs through a hydroxyl group of a spacer, the amino acid or one of the amino acids of the dipeptide or tripeptide can be an amino acid of Formula II. In a specific exemplary embodiment, the amino acid is Ser.

When acylation occurs through a thiol group of a spacer, the amino acid or one of the amino acids of the dipeptide or tripeptide can be an amino acid of Formula III. In a specific exemplary embodiment, the amino acid is Cys.

In some embodiments, the spacer is a hydrophilic bifunctional spacer. In certain embodiments, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.).

In some embodiments, the spacer is a hydrophobic bifunctional spacer. Hydrophobic bifunctional spacers are known in the art. See, e.g., Bioconjugate Techniques, G. T. Hermanson (Academic Press, San Diego, Calif., 1996), which is incorporated by reference in its entirety. In certain embodiments, the hydrophobic bifunctional spacer comprises two or more reactive groups, e.g., an amine, a hydroxyl, a thiol, and a carboxyl group or any combinations thereof. In certain embodiments, the hydrophobic bifunctional spacer comprises a hydroxyl group and a carboxylate. In other embodiments, the hydrophobic bifunctional spacer comprises an amine group and a carboxylate. In other embodiments, the hydrophobic bifunctional spacer comprises a thiol group and a carboxylate. Suitable hydrophobic bifunctional spacers comprising a carboxylate and a hydroxyl group or a thiol group are known in the art and include, for example, 8-hydroxyoctanoic acid and 8-mercaptooctanoic acid.

The spacer (e.g., amino acid, dipeptide, tripeptide, hydrophilic bifunctional spacer, or hydrophobic bifunctional spacer) in specific embodiments is 3 to 10 atoms (e.g., 6 to 10 atoms, (e.g., 6, 7, 8, 9, or 10 atoms) in length. In more specific embodiments, the spacer is about 3 to 10 atoms (e.g., 6 to 10 atoms) in length and the acyl group is a C12 to C18 fatty acyl group, e.g., C14 fatty acyl group, C16 fatty acyl group, such that the total length of the spacer and acyl group is 14 to 28 atoms, e.g., about 14, 15, 16, 17, 18, 19, 20, 21, 22, 23, 24, 25, 26, 27, or 28 atoms. In some embodiments, the length of the spacer and acyl group is 17 to 28 (e.g., 19 to 26, 19 to 21) atoms.

In accordance with certain foregoing embodiments, the bifunctional spacer can be a synthetic or naturally occurring amino acid (including, but not limited to, any of those described herein) comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. Each amino acid of the dipeptide or tripeptide spacer can be the same as or different from the other amino acid(s) of the dipeptide or tripeptide and can be independently selected from the group consisting of: naturally-occurring or coded and/or non-coded or non-naturally occurring amino acids, including, for example, any of the D or L isomers of the naturally-occurring amino acids (Ala, Cys, Asp, Glu, Phe, Gly, His, Ile, Lys, Leu, Met, Asn, Pro, Arg, Ser, Thr, Val, Trp, Tyr), or any D or L isomers of the non-naturally occurring or non-coded amino acids selected from the group consisting of: β-alanine (β-Ala), N-α-methyl-alanine (Me-Ala), aminobutyric acid (Abu), γ-aminobutyric acid (γ-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl)alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ-Glu), homoserine (Hse), hydroxyproline (Hyp), isoleucine N-methoxy-N-methyl amide, methyl-isoleucine (MeIle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met(O₂)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-Cl)), 4-fluorophenylalanine (Phe(4-F)), 4-nitrophenylalanine (Phe(4-NO₂)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), O-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4,-tetrahydro-isoquinoline-3-carboxylic acid (Tic), tetrahydropyranglycine, thienylalanine (Thi), O-benzyl-phosphotyrosine, O-Phosphotyrosine, methoxytyrosine, ethoxytyrosine, O-(bis-dimethylamino-phosphono)-tyrosine, tyrosine sulfate tetrabutylamine, methyl-valine (MeVal), and alkylated 3-mercaptopropionic acid.

In some embodiments, the spacer comprises an overall negative charge, e.g., comprises one or two negative-charged amino acids. In some embodiments, the dipeptide is not any of the dipeptides of general structure A-B, wherein A is selected from the group consisting of Gly, Gln, Ala, Arg, Asp, Asn, Be, Leu, Val, Phe, and Pro, wherein B is selected from the group consisting of Lys, His, Trp. In some embodiments, the dipeptide spacer is selected from the group consisting of: Ala-Ala, β-Ala- β-Ala, Leu-Leu, Pro-Pro, γ-aminobutyric acid- γ-aminobutyric acid, Glu-Glu, and γ-Glu- γ-Glu.

In some exemplary embodiments, the GLP-1 analog is modified to comprise an acyl group by acylation of an amine, hydroxyl, or thiol of a spacer, which spacer is attached to a side chain of an amino acid at position 10, 20, 24, or 29, or at the C-terminal amino acid of the GLP-1 analog.

In yet more specific embodiments, the acyl group is attached to the amino acid at position 10 of the GLP-1 analog and the length of the spacer and acyl group is 14 to 28 atoms. The amino acid at position 10, in some aspects, is an amino acid of Formula I, e.g., Lys, or a disubstituted amino acid related to Formula I. In more specific embodiments, the GLP-1 analog lacks an intramolecular bridge, e.g., a covalent intramolecular bridge. The GLP-1 analog, for example, can be a GLP-1 analog comprising one or more alpha, alpha-disubstituted amino acids, e.g., Aib, for stabilizing the alpha helix of the analog.

Suitable methods of peptide acylation via amines, hydroxyls, and thiols are known in the art. See, for example, Miller, Biochem Biophys Res Commun 218: 377-382 (1996); Shimohigashi and Stammer, Int J Pept Protein Res 19: 54-62 (1982); and Previero et al., Biochim Biophys Acta 263: 7-13 (1972) (for methods of acylating through a hydroxyl); and San and Silvius, J Pept Res 66: 169-180 (2005) (for methods of acylating through a thiol); Bioconjugate Chem. “Chemical Modifications of Proteins: History and Applications” pages 1, 2-12 (1990); Hashimoto et al., Pharmacuetical Res. “Synthesis of Palmitoyl Derivatives of Insulin and their Biological Activity” Vol. 6, No: 2 pp. 171-176 (1989).

The acyl group of the acylated amino acid can be of any size, e.g., any length carbon chain, and can be linear or branched. In some specific embodiments, the acyl group is a C4 to C30 fatty acid. For example, the acyl group can be any of a C4 fatty acid, C6 fatty acid, C8 fatty acid, C10 fatty acid, C12 fatty acid, C14 fatty acid, C16 fatty acid, C18 fatty acid, C20 fatty acid, C22 fatty acid, C24 fatty acid, C26 fatty acid, C28 fatty acid, or a C30 fatty acid. In some embodiments, the acyl group is a C8 to C20 fatty acid, e.g., a C14 fatty acid or a C16 fatty acid.

In an alternative embodiment, the acyl group is a bile acid. The bile acid can be any suitable bile acid, including, but not limited to, cholic acid, chenodeoxycholic acid, deoxycholic acid, lithocholic acid, taurocholic acid, glycocholic acid, and cholesterol acid.

In some embodiments, the GLP-1 analog comprises an acylated amino acid by acylation of a long chain alkane by the GLP-1 analog. In specific aspects, the long chain alkane comprises an amine, hydroxyl, or thiol group (e.g., octadecylamine, tetradecanol, and hexadecanethiol) which reacts with a carboxyl group, or activated form thereof, of the GLP-1 analog. The carboxyl group, or activated form thereof, of the GLP-1 analog can be part of a side chain of an amino acid (e.g., glutamic acid, aspartic acid) of the GLP-1 analog or can be part of the analog backbone.

In certain embodiments, the GLP-1 analog is modified to comprise an acyl group by acylation of the long chain alkane by a spacer which is attached to the GLP-1 analog. In specific aspects, the long chain alkane comprises an amine, hydroxyl, or thiol group which reacts with a carboxyl group, or activated form thereof, of the spacer. Suitable spacers comprising a carboxyl group, or activated form thereof, are described herein and include, for example, bifunctional spacers, e.g., amino acids, dipeptides, tripeptides, hydrophilic bifunctional spacers and hydrophobic bifunctional spacers.

As used herein, the term “activated form of a carboxyl group” refers to a carboxyl group with the general formula R(C═O)X, wherein X is a leaving group and R is the GLP-1 analog or the spacer. For example, activated forms of a carboxyl groups may include, but are not limited to, acyl chlorides, anhydrides, and esters. In some embodiments, the activated carboxyl group is an ester with a N-hydroxysuccinimide ester (NHS) leaving group.

With regard to these aspects, in which a long chain alkane is acylated by the GLP-1 analog or the spacer, the long chain alkane may be of any size and can comprise any length of carbon chain. The long chain alkane can be linear or branched. In certain aspects, the long chain alkane is a C4 to C30 alkane. For example, the long chain alkane can be any of a C4 alkane, C6 alkane, C8 alkane, C10 alkane, C12 alkane, C14 alkane, C16 alkane, C18 alkane, C20 alkane, C22 alkane, C24 alkane, C26 alkane, C28 alkane, or a C30 alkane. In some embodiments, the long chain alkane comprises a C8 to C20 alkane, e.g., a C14 alkane, C16 alkane, or a C18 alkane.

Also, in some embodiments, an amine, hydroxyl, or thiol group of the GLP-1 analog is acylated with a cholesterol acid. In a specific embodiment, the GLP-1 analog is linked to the cholesterol acid through an alkylated des-amino Cys spacer, i.e., an alkylated 3-mercaptopropionic acid spacer. The alkylated des-amino Cys spacer can be, for example, a des-amino-Cys spacer comprising a dodecaethylene glycol moiety. In one embodiment, the GLP-1 analog comprises the structure:

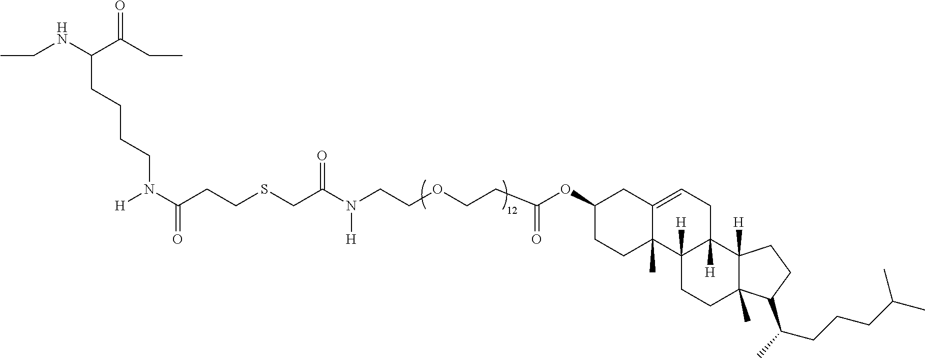

The acylated GLP-1 analogs described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein. In this regard, the acylated GLP-1 analog can comprise SEQ ID NO: 38, including any of the modifications described herein, in which at least one of the amino acids at position 10, 20, 24, and 29 of the analog comprises an acyl group and at least one of the amino acids at position 16, 17, 21, 24, or 29, a position within a C-terminal extension, or the C-terminal amino acid are modified to a Cys, Lys, Orn, homo-Cys, or Ac-Phe, and the side chain of the amino acid is covalently bonded to a hydrophilic moiety (e.g., PEG). In some embodiments, the acyl group is attached to position 10, optionally via a spacer comprising Cys, Lys, Orn, homo-Cys, or Ac-Phe, and the hydrophilic moiety is incorporated at a Cys residue at position 24.

Alternatively, the acylated GLP-1 analog can comprise a spacer, wherein the spacer is both acylated and modified to comprise the hydrophilic moiety. Nonlimiting examples of suitable spacers include a spacer comprising one or more amino acids selected from the group consisting of Cys, Lys, Orn, homo-Cys, and Ac-Phe.

In certain embodiments, the GLP-1 agonist peptide comprises an amino acid comprising a side chain covalently attached, optionally, through a spacer, to an acyl group or an alkyl group, which acyl group or alkyl group is non-native to a naturally-occurring amino acid. In one embodiment the acylated glucagon peptide comprises the sequence of SEQ ID NO: 37. In one embodiment the covalently linked acyl or alkyl group has a carboxylate at its free end. The acyl group in some embodiments is a C4 to C30 fatty acyl group, optionally with carboxylate groups at each end. In one embodiment the GLP-1 agonist peptide comprises a covalently linked, optionally through a spacer, acyl group, wherein the alkyl group is a C4 to C30 alkyl optionally with a carboxylate at its free end. In specific aspects, the acyl group or alkyl group is covalently attached to the side chain of the amino acid at position 10. In one embodiment the acyl group is a C16, C18 or C20 acyl group optionally with a carboxylate at its free end when linked to the GLP-1 agonist peptide.

Alkylation

In accordance with some embodiments, the GLP-1 analog comprises an alkylated amino acid (e.g., a non-coded alkylated amino acid (e.g., an amino acid comprising an alkyl group which is non-native to a naturally-occurring amino acid)). Without being held to any particular theory, it is believed that alkylation of GLP-1 analogs achieve similar, if not the same, effects as acylation of the GLP-1 analogs, e.g., a prolonged half-life in circulation, a delayed onset of action, an extended duration of action, an improved resistance to proteases, such as DPP-IV, and increased potency at the GLP-1 and glucagon receptors.

Alkylation can be carried out at any positions within the GLP-1 analog, including any of the positions described herein as a site for acylation, including but not limited to, any of amino acid positions 1-29, an amino acid position C-terminal to the 29^{th} residue, e.g., 30, 31, 32, 33, 34, 35, 36, 37, 38, 39, 40, 41, 42, 43, 44, 45, 46, 47, etc., at a position within a C-terminal extension, or at the C-terminus, provided that the GLP-1 activity is retained. Nonlimiting examples include positions 5, 7, 10, 11, 12, 13, 14, 16, 17, 18, 19, 20, 21, 24, 27, 28, or 29. In exemplary embodiments, the GLP-1 analog comprises an alkylated amino acid at one or more positions selected from the group consisting of: 9, 10, 12, 16, and 20. In exemplary embodiments, the GLP-1 analog comprises an alkylated amino acid at one or more positions selected from the group consisting of: 10, 12, and 16. In exemplary embodiments, the GLP-1 analog comprises an alkylated amino acid at one or more positions selected from the group consisting of: 9, 10, 12, 16, and 20. In exemplary embodiments, the GLP-1 analog comprises an alkylated amino acid at one or more positions 10 and 12. In exemplary embodiments, the GLP-1 analog comprises an alkylated amino acid at position 12. In exemplary embodiments, the GLP-1 analog comprises a C-terminal extension and an alkylated amino acid at one or more positions selected from the group consisting of 9, 10, 12, 16, 20, and 37-43 (e.g., 40). The alkyl group can be covalently linked directly to an amino acid of the GLP-1 analog, or indirectly to an amino acid of the GLP-1 analog via a spacer, wherein the spacer is positioned between the amino acid of the GLP-1 analog and the alkyl group. GLP-1 analog may be alkylated at the same amino acid position where a hydrophilic moiety is linked, or at a different amino acid position. Nonlimiting examples include alkylation at position 10 and pegylation at one or more positions in the C-terminal portion of the GLP-1 analog, e.g., position 24, 28 or 29, within a C-terminal extension, or at the C-terminus (e.g., through adding a C-terminal Cys).

In specific aspects, the GLP-1 analog is modified to comprise an alkyl group by direct alkylation of an amine, hydroxyl, or thiol of a side chain of an amino acid of the GLP-1 analog. In some embodiments, alkylation is at position 10, 20, 24, or 29 of the GLP-1 analog. In this regard, the alkylated GLP-1 analog can comprise the amino acid sequence of SEQ ID NO: 38, or a modified amino acid sequence thereof comprising one or more of the amino acid modifications described herein, with at least one of the amino acids at positions 10, 20, 24, and 29 modified to any amino acid comprising a side chain amine, hydroxyl, or thiol. In some specific embodiments, the direct alkylation of the GLP-1 analog occurs through the side chain amine, hydroxyl, or thiol of the amino acid at position 10.

In some embodiments, the amino acid comprising a side chain amine is an amino acid of Formula I. In some exemplary embodiments, the amino acid of Formula I, is the amino acid wherein n is 4 (Lys) or n is 3 (Orn).

In other embodiments, the amino acid comprising a side chain hydroxyl is an amino acid of Formula II. In some exemplary embodiments, the amino acid of Formula II is the amino acid wherein n is 1 (Ser).

In yet other embodiments, the amino acid comprising a side chain thiol is an amino acid of Formula III. In some exemplary embodiments, the amino acid of Formula III is the amino acid wherein n is 1 (Cys).

In yet other embodiments, the amino acid comprising a side chain amine, hydroxyl, or thiol is a disubstituted amino acid comprising the same structure of Formula I, Formula II, or Formula III, except that the hydrogen bonded to the alpha carbon of the amino acid of Formula I, Formula II, or Formula III is replaced with a second side chain.

In some embodiments, the alkylated GLP-1 analog comprises a spacer between the analog and the alkyl group. In some embodiments, the GLP-1 analog is covalently bound to the spacer, which is covalently bound to the alkyl group. In some exemplary embodiments, the GLP-1 analog is modified to comprise an alkyl group by alkylation of an amine, hydroxyl, or thiol of a spacer, which spacer is attached to a side chain of an amino acid at position 10, 20, 24, or 29 of the GLP-1 analog. The amino acid to which the spacer is attached can be any amino acid comprising a moiety which permits linkage to the spacer. For example, an amino acid comprising a side chain NH₂, —OH, or —COOH (e.g., Lys, Orn, Ser, Asp, or Glu) is suitable. In this respect, the alkylated GLP-1 analog can comprise a modified amino acid sequence of SEQ ID NO: 38, comprising one or more of the amino acid modifications described herein, with at least one of the amino acids at positions 10, 20, 24, and 29 modified to any amino acid comprising a side chain amine, hydroxyl, or carboxylate.

In some embodiments, the spacer is an amino acid comprising a side chain amine, hydroxyl, or thiol or a dipeptide or tripeptide comprising an amino acid comprising a side chain amine, hydroxyl, or thiol.

When alkylation occurs through an amine group of a spacer, the alkylation can occur through the alpha amine of an amino acid or a side chain amine. In the instance in which the alpha amine is alkylated, the amino acid of the spacer can be any amino acid. For example, the amino acid of the spacer can be a hydrophobic amino acid, e.g., Gly, Ala, Val, Leu, Ile, Trp, Met, Phe, Tyr, 6-amino hexanoic acid, 5-aminovaleric acid, 7-aminoheptanoic acid, and 8-aminooctanoic acid. Alternatively, the amino acid of the spacer can be an acidic residue, e.g., Asp and Glu, provided that the alkylation occurs on the alpha amine of the acidic residue. In the instance in which the side chain amine of the amino acid of the spacer is alkylated, the amino acid of the spacer is an amino acid comprising a side chain amine, e.g., an amino acid of Formula I (e.g., Lys or Orn). In this instance, it is possible for both the alpha amine and the side chain amine of the amino acid of the spacer to be alkylated, such that the GLP-1 analog is dialkylated. Embodiments of the invention include such dialkylated molecules.

When alkylation occurs through a hydroxyl group of a spacer, the amino acid or one of the amino acids of the dipeptide or tripeptide can be an amino acid of Formula II. In a specific exemplary embodiment, the amino acid is Ser.

When alkylation occurs through a thiol group of spacer, the amino acid or one of the amino acids of the dipeptide or tripeptide can be an amino acid of Formula III. In a specific exemplary embodiment, the amino acid is Cys.

In some embodiments, the spacer is a hydrophilic bifunctional spacer. In certain embodiments, the hydrophilic bifunctional spacer comprises two or more reactive groups, e.g., an amine, a hydroxyl, a thiol, and a carboxyl group or any combinations thereof. In certain embodiments, the hydrophilic bifunctional spacer is comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.).

In some embodiments, the spacer is a hydrophobic bifunctional spacer. In certain embodiments, the hydrophobic bifunctional spacer comprises two or more reactive groups, e.g., an amine, a hydroxyl, a thiol, and a carboxyl group or any combinations thereof. In certain embodiments, the hydrophobic bifunctional spacer comprises a hydroxyl group and a carboxylate. In other embodiments, the hydropholic bifunctional spacer comprises an amine group and a carboxylate. In other embodiments, the hydropholic bifunctional spacer comprises a thiol group and a carboxylate. Suitable hydrophobic bifunctional spacers comprising a carboxylate and a hydroxyl group or a thiol group are known in the art and include, for example, 8-hydroxyoctanoic acid and 8-mercaptooctanoic acid.

The spacer (e.g., amino acid, dipeptide, tripeptide, hydrophilic bifunctional spacer, or hydrophobic bifunctional spacer) in specific embodiments is 3 to 10 atoms (e.g., 6 to 10 atoms, (e.g., 6, 7, 8, 9, or 10 atoms)) in length. In more specific embodiments, the spacer is about 3 to 10 atoms (e.g., 6 to 10 atoms) in length and the alkyl is a C12 to C18 alkyl group, e.g., C14 alkyl group, C16 alkyl group, such that the total length of the spacer and alkyl group is 14 to 28 atoms, e.g., about 14, 15, 16, 17, 18, 19, 20, 21, 22, 23, 24, 25, 26, 27, or 28 atoms. In some embodiments, the length of the spacer and alkyl is 17 to 28 (e.g., 19 to 26, 19 to 21) atoms.

In accordance with certain foregoing embodiments, the bifunctional spacer can be a synthetic or non-naturally occurring or non-coded amino acid comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. The dipeptide or tripeptide spacer can be composed of naturally-occurring or coded and/or non-coded or non-naturally occurring amino acids, including, for example, any of the amino acids taught herein. In some embodiments, the spacer comprises an overall negative charge, e.g., comprises one or two negative-charged amino acids. In some embodiments, the dipeptide spacer is selected from the group consisting of: Ala-Ala, β-Ala- β-Ala, Leu-Leu, Pro-Pro, γ-aminobutyric acid- γ-aminobutyric acid, and γ-Glu- γ-Glu.

Suitable methods of peptide alkylation via amines, hydroxyls, and thiols are known in the art. For example, a Williamson ether synthesis can be used to form an ether linkage between a hydroxyl group of the GLP-1 analog and the alkyl group. Also, a nucleophilic substitution reaction of the peptide with an alkyl halide can result in any of an ether, thioether, or amino linkage.

The alkyl group of the alkylated GLP-1 analog can be of any size, e.g., any length carbon chain, and can be linear or branched. In some embodiments, the alkyl group is a C4 to C30 alkyl. For example, the alkyl group can be any of a C4 alkyl, C6 alkyl, C8 alkyl, C10 alkyl, C12 alkyl, C14 alkyl, C16 alkyl, C18 alkyl, C20 alkyl, C22 alkyl, C24 alkyl, C26 alkyl, C28 alkyl, or a C30 alkyl. In some embodiments, the alkyl group is a C8 to C20 alkyl, e.g., a C14 alkyl or a C16 alkyl.

In some specific embodiments, the alkyl group comprises a steroid moiety of a bile acid, e.g., cholic acid, chenodeoxycholic acid, deoxycholic acid, lithocholic acid, taurocholic acid, glycocholic acid, and cholesterol acid.

In some embodiments of the disclosure, the GLP-1 analog comprises an alkylated amino acid by reacting a nucleophilic, long chain alkane with the GLP-1 analog, wherein the GLP-1 analog comprises a leaving group suitable for nucleophilic substitution. In specific aspects, the nucleophilic group of the long chain alkane comprises an amine, hydroxyl, or thiol group (e.g., octadecylamine, tetradecanol, and hexadecanethiol). The leaving group of the GLP-1 analog can be part of a side chain of an amino acid or can be part of the peptide backbone. Suitable leaving groups include, for example, N-hydroxysuccinimide, halogens, and sulfonate esters.

In certain embodiments, the GLP-1 analog is modified to comprise an alkyl group by reacting the nucleophilic, long chain alkane with a spacer which is attached to the GLP-1 analog, wherein the spacer comprises the leaving group. In specific aspects, the long chain alkane comprises an amine, hydroxyl, or thiol group. In certain embodiments, the spacer comprising the leaving group can be any spacer discussed herein, e.g., amino acids, dipeptides, tripeptides, hydrophilic bifunctional spacers and hydrophobic bifunctional spacers further comprising a suitable leaving group.

With regard to these aspects of the disclosure, in which a long chain alkane is alkylated by the GLP-1 analog or the spacer, the long chain alkane may be of any size and can comprise any length of carbon chain. The long chain alkane can be linear or branched. In certain aspects, the long chain alkane is a C4 to C30 alkane. For example, the long chain alkane can be any of a C4 alkane, C6 alkane, C8 alkane, C10 alkane, C12 alkane, C14 alkane, C16 alkane, C18 alkane, C20 alkane, C22 alkane, C24 alkane, C26 alkane, C28 alkane, or a C30 alkane. In some embodiments, the long chain alkane comprises a C8 to C20 alkane, e.g., a C14 alkane, C16 alkane, or a C18 alkane.

Also, in some embodiments, alkylation can occur between the GLP-1 analog and a cholesterol moiety. For example, the hydroxyl group of cholesterol can displace a leaving group on the long chain alkane to form a cholesterol-GLP-1 analog product.

The alkylated GLP-1 analogs described herein can be further modified to comprise a hydrophilic moiety. In some specific embodiments the hydrophilic moiety can comprise a polyethylene glycol (PEG) chain. The incorporation of a hydrophilic moiety can be accomplished through any suitable means, such as any of the methods described herein. In this regard, the alkylated GLP-1 analog can comprise a modified SEQ ID NO: 38 comprising one or more of the amino acid modifications described herein, in which at least one of the amino acids at position 10, 20, 24, and 29 comprise an alkyl group and at least one of the amino acids at position 16, 17, 21, 24, and 29, a position within a C-terminal extension or the C-terminal amino acid are modified to a Cys, Lys, Orn, homo-Cys, or Ac-Phe, and the side chain of the amino acid is covalently bonded to a hydrophilic moiety (e.g., PEG). In some embodiments, the alkyl group is attached to position 10, optionally via a spacer comprising Cys, Lys, Orn, homo-Cys, or Ac-Phe, and the hydrophilic moiety is incorporated at a Cys residue at position 24.

Alternatively, the alkylated GLP-1 analog can comprise a spacer, wherein the spacer is both alkylated and modified to comprise the hydrophilic moiety. Nonlimiting examples of suitable spacers include a spacer comprising one or more amino acids selected from the group consisting of Cys, Lys, Orn, homo-Cys, and Ac-Phe.

Stabilization of the Alpha Helix and Alpha Helix Promoting Amino Acids

Without being bound to any particular theory, the GLP-1 analogs described herein comprise a helical structure, e.g., an alpha helix. In some embodiments, the GLP-1 analog comprises amino acids which stabilize the alpha helical structure. Accordingly, in some aspects, the GLP-1 analog comprises one or more alpha helix promoting amino acids. As used herein, the term “alpha helix promoting amino acid” refers to an amino acid which provides increased stability to an alpha helix of the GLP-1 analog of which it is a part. Alpha helix promoting amino acids are known in the art. See, for example, Lyu et al., Proc Natl Acad Sci U.S.A. 88: 5317-5320 (1991); Branden & Tooze, Introduction to Protein Structure, Garland Publishing, New York, N.Y., 1991; Fasman, Prediction of Protein Structure and the Principles of Protein Conformation, ed. Fasman, Plenum, N Y, 1989). Suitable alpha helix promoting amino acids for purposes herein include, but are not limited to: alanine, norvaline, norleucine, alpha aminobutyric acid, alpha-aminoisobutyric acid, leucine, isoleucine, valine, and the like. In some embodiments, the alpha helix promoting amino acid is any amino acid which is part of an alpha helix found in a naturally-occurring protein, e.g., Leu, Phe, Ala, Met, Gly, Ile, Ser, Asn, Glu, Asp, Lys, Arg.

In some embodiments, the alpha helix promoting amino acid provides more stability to the alpha helix as compared to glycine or alanine. In some embodiments, the alpha helix promoting amino acid is an alpha, alpha di-substituted amino acid.

Alpha Helix: Position of Alpha Helix Promoting Amino Acids

In some embodiments, the GLP-1 analog comprises an amino acid sequence which is similar to native GLP-1 (SEQ ID NO: 44) or SEQ ID NO: 38 wherein the GLP-1 analog comprises at least one alpha helix promoting amino acid. In some embodiments, the alpha helix promoting amino acid is located at any of positions 12 to 29 (according to the numbering of the GLP-1 analog of SEQ ID NO: 38). In some embodiments, the GLP-1 analog comprises a modified amino acid sequence of SEQ ID NO: 38 and comprises at least one alpha helix promoting amino acid, e.g., at one or more of positions 12, 13, 14, 15, 16, 17, 18, 19, 20, 21, 22, 23, 24, 25, 26, 27, 28, 29. In some embodiments, the GLP-1 analog comprises an alpha helix promoting amino acid at one, two, three, or all of positions 16, 17, 20, and 21.

Alpha Helix: Alpha, Alpha Di-Substituted Amino Acids

In some embodiments, the alpha helix promoting amino acid is an alpha, alpha di-substituted amino acid. In specific embodiments, the alpha, alpha di-substituted amino acid comprises R¹ and R², each of which is bonded to the alpha carbon, wherein each of R¹ and R² is independently selected from the group consisting of C1-C4 alkyl, optionally substituted with a hydroxyl, amide, thiol, halo, or R¹ and R² together with the alpha carbon to which they are attached form a ring (e.g., a C3-C8 ring). In some embodiments, each of R¹ and R² is selected from the group consisting of: methyl, ethyl, propyl, and n-butyl, or R¹ and R² together form a cyclooctane or cycloheptane (e.g., 1-aminocyclooctane-1-carboxylic acid). In some embodiments, R¹ and R² are the same. In some embodiments, R¹ is different from R². In certain aspects, each of R¹ and R² is a C1-C4 alkyl. In some aspects, each of R¹ and R² is a C1 or C2 alkyl. In some embodiments, each of R¹ and R² is methyl, such that the alpha, alpha disubstituted amino acid is alpha-aminoisobutyric acid (Aib).

In some aspects, the GLP-1 analogs described herein comprises one or more alpha, alpha di-substituted amino acids and the GLP-1 analogs specifically lack a covalent intramolecular bridge (e.g., a lactam), since the alpha, alpha disubstituted amino acid is capable of stabilizing the alpha helix in the absence of a covalent bridge. In some aspects, the GLP-1 analog comprises one or more alpha, alpha di-substituted amino acids at the C-terminus (around positions 12-29). In some embodiments, one, two, three, four or more of positions 16, 17, 18, 19, 20, 21, 24, 28, or 29 of the GLP-1 analog is substituted with an α, α-disubstituted amino acid, e.g., amino iso-butyric acid (Aib), an amino acid disubstituted with the same or a different group selected from methyl, ethyl, propyl, and n-butyl, or with a cyclooctane or cycloheptane (e.g., 1-aminocyclooctane-1-carboxylic acid). For example, substitution of position 16 with Aib enhances GLP-1 activity, in the absence of an intramolecular bridge, e.g., a non-covalent intramolecular bridge (e.g., a salt bridge) or a covalent intramolecular bridge (e.g., a lactam). In some embodiments, one, two, three or more of positions 16, 20, 21 or 24 are substituted with Aib. In specific embodiments, one or both of the amino acids corresponding to positions 2, 16, of the GLP-1 analog of SEQ ID NO: 38 are substituted with an alpha, alpha disubstituted amino acid such as Aib.

In accordance with some embodiments, the GLP-1 analog lacking an intramolecular bridge comprises one or more substitutions within amino acid positions 12-29 with an α, α-disubstituted amino acid and an acyl or alkyl group covalently attached to the side chain of the amino acid at position 10 of the GLP-1 analog. In specific embodiments, the acyl or alkyl group is not naturally occurring on an amino acid. In certain aspects, the acyl or alkyl group is non-native to the amino acid at position 10. Such acylated or alkylated GLP-1 peptides lacking an intramolecular bridge exhibit enhanced activity at the GLP-1 and glucagon receptors as compared to the non-acylated counterpart peptides. Further enhancement in activity at the GLP-1 and glucagon receptors can be achieved by the acylated GLP-1 peptides lacking an intramolecular bridge by incorporating a spacer between the acyl or alkyl group and the side chain of the amino acid at position 10 of the analog. Acylation and alkylation, with or without incorporating spacers, are further described herein.

Alpha Helix: Intramolecular Bridges

In some embodiments, the alpha helix promoting amino acid is an amino acid which is linked to another amino acid of the GLP-1 analog via an intramolecular bridge. In such embodiments, each of these two amino acids linked via an intramolecular bridge is considered an alpha helix promoting amino acid. In some embodiments, the GLP-1 analog comprises one or two intramolecular bridges. In some specific embodiments, the GLP-1 analog comprises one intramolecular bridge in combination with at least one other alpha helix promoting amino acid, e.g., an alpha, alpha-disubstituted amino acid.

In some embodiments, the intramolecular bridge is a bridge which connects two parts of the GLP-1 analog via noncovalent bonds, including, for example, van der Waals interactions, hydrogen bonds, ionic bonds, hydrophobic interactions, dipole-dipole interactions, and the like. In this regard, the GLP-1 analog in certain aspects comprises a non-covalent intramolecular bridge. In some embodiments, the non-covalent intramolecular bridge is a salt bridge.

In some embodiments, the intramolecular bridge is a bridge which connects two parts of the analog via covalent bonds. In this regard, the GLP-1 analog in certain aspects comprises a covalent intramolecular bridge.

In some embodiments, the intramolecular bridge (e.g., non-covalent intramolecular bridge, covalent intramolecular bridge) is formed between two amino acids that are 3 amino acids apart, e.g., amino acids at positions i and i+4, wherein i is any integer between 12 and 25 (e.g., 12, 13, 14, 15, 16, 17, 18, 19, 20, 21, 22, 23, 24, and 25). More particularly, the side chains of the amino acid pairs 12 and 16, 16 and 20, 20 and 24 or 24 and 28 (amino acid pairs in which i=12, 16, 20, or 24) are linked to one another and thus stabilize the GLP-1 alpha helix. Alternatively, i can be 17. In some specific embodiments, the GLP-1 analog comprises an intramolecular bridge between amino acids 17 and 21. In some specific embodiments, the GLP-1 analog comprises an intramolecular bridge between the amino acids at positions 16 and 20 or 12 and 16 and a second intramolecular bridge between the amino acids at positions 17 and 21. GLP-1 analogs comprising one or more intramolecular bridges are contemplated herein. In specific embodiments, wherein the amino acids at positions i and i+4 are joined by an intramolecular bridge, the size of the linker is about 8 atoms, or about 7-9 atoms.

In other embodiments, the intramolecular bridge is formed between two amino acids that are two amino acids apart, e.g., amino acids at positions j and j+3, wherein j is any integer between 12 and 26 (e.g., 12, 13, 14, 15, 16, 17, 18, 19, 20, 21, 22, 23, 24, 25, and 26). In some specific embodiments, j is 17. In specific embodiments, wherein amino acids at positions j and j+3 are joined by an intramolecular bridge, the size of the linker is about 6 atoms, or about 5 to 7 atoms.

In yet other embodiments, the intramolecular bridge is formed between two amino acids that are 6 amino acids apart, e.g., amino acids at positions k and k+7, wherein k is any integer between 12 and 22 (e.g., 12, 13, 14, 15, 16, 17, 18, 19, 20, 21, and 22). In some specific embodiments, k is 12, 13, or 17. In an exemplary embodiment, k is 17.

Alpha Helix: Amino Acids Involved in Intramolecular Bridges

Examples of amino acid pairings that are capable of bonding (covalently or non-covalently) to form a six-atom linking bridge include Orn and Asp, Glu and an amino acid of Formula I, wherein n is 2, and homoglutamic acid and an amino acid of Formula I, wherein n is 1, wherein Formula I is:

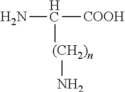

wherein n=1 to 4

[Formula I]

Examples of amino acid pairings that are capable of bonding to form a seven-atom linking bridge include Orn-Glu (lactam ring); Lys-Asp (lactam); or Homoser-Homoglu (lactone). Examples of amino acid pairings that may form an eight-atom linker include Lys-Glu (lactam); Homolys-Asp (lactam); Orn-Homoglu (lactam); 4-aminoPhe-Asp (lactam); or Tyr-Asp (lactone). Examples of amino acid pairings that may form a nine-atom linker include Homolys-Glu (lactam); Lys-Homoglu (lactam); 4-aminoPhe-Glu (lactam); or Tyr-Glu (lactone). Any of the side chains on these amino acids may additionally be substituted with additional chemical groups, so long as the three-dimensional structure of the alpha-helix is not disrupted. One of ordinary skill in the art can envision alternative pairings or alternative amino acid analogs, including chemically modified derivatives, that would create a stabilizing structure of similar size and desired effect. For example, a homocysteine-homocysteine disulfide bridge is 6 atoms in length and may be further modified to provide the desired effect.

Even without covalent linkage, the amino acid pairings described above (or similar pairings that one of ordinary skill in the art can envision) may also provide added stability to the alpha-helix through non-covalent bonds, for example, through formation of salt bridges or hydrogen-bonding interactions. Accordingly, salt bridges may be formed between: Orn and Glu; Lys and Asp; Homo-serine and Homo-glutamate; Lys and Glu; Asp and Arg; Homo-Lys and Asp; Orn and Homo-Glutamate; 4-aminoPhe and Asp; Tyr and Asp; Homo-Lys and Glu; Lys and Homo-Glu; 4-aminoPhe and Glu; or Tyr and Glu. In some embodiments, the analog comprises a salt bridge between any of the following pairs of amino acids: Orn and Glu; Lys and Asp; Lys and Glu; Asp and Arg; Homo-Lys and Asp; Orn and Homo-Glutamate; Homo-Lys and Glu; and Lys and Homo-Glu. Salt bridges may be formed between other pairs of oppositely charged side chains. See, e.g., Kallenbach et al., Role of the Peptide Bond in Protein Structure and Folding, in The Amide Linkage: Structural Significance in Chemistry, Biochemistry, and Materials Science, John Wiley & Sons, Inc. (2000).

In some embodiments, the non-covalent intramolecular bridge is a hydrophobic bridge. In accordance with one embodiment, the alpha helix of the analog is stabilized through the incorporation of hydrophobic amino acids at positions j and j+3 or i and i+4. For instance, i can be Tyr and i+4 can be either Val or Leu; i can be Phe and i+4 can be Met; or i can be Phe and i+4 can be Be. It should be understood that, for purposes herein, the above amino acid pairings can be reversed, such that the indicated amino acid at position i could alternatively be located at i+4, while the i+4 amino acid can be located at the i position. It should also be understood that suitable amino acid pairings can be formed for j and j+3.

Alpha Helix: Covalent Intramolecular Bridge

In some embodiments, the covalent intramolecular bridge is a lactam ring or lactam bridge. The size of the lactam ring can vary depending on the length of the amino acid side chains, and in one embodiment the lactam is formed by linking the side chains of an ornithine to an aspartic acid side chain. Lactam bridges and methods of making the same are known in the art. See, for example, Houston, Jr., et al., J Peptide Sci 1: 274-282 (2004). In some embodiments, the analog comprises a modified sequence of SEQ ID NO: 44 or SEQ ID NO: 38 and a lactam bridge between i and i+4, wherein i is as defined herein above. In some embodiments, the GLP-1 analog comprises two lactam bridges: one between the amino acids at positions 16 and 20 and another between the amino acids at positions 17 and 21. In some embodiments, the GLP-1 analog comprises one lactam bridge and one salt bridge. Further exemplary embodiments include the following pairings, optionally with a lactam bridge: Glu at position 12 with Lys at position 16; Lys at position 12 with Glu at position 16; Glu at position 16 with Lys at position 20; Lys at position 16 with Glu at position 20; Glu at position 20 with Lys at position 24; Lys at position 20 with Glu at position 24; Glu at position 24 with Lys at position 28; Lys at position 24 with Glu at position 28.

In some embodiments, the covalent intramolecular bridge is a lactone. Suitable methods of making a lactone bridge are known in the art. See, for example, Sheehan et al., J Am Chem Soc 95: 875-879 (1973).

In some aspects, olefin metathesis is used to cross-link one or two turns of the alpha helix of the analog using an all-hydrocarbon cross-linking system. The GLP-1 analog in this instance comprises α-methylated amino acids bearing olefinic side chains of varying length and configured with either R or S stereochemistry at the j and j+3 or i and i+4 positions. In some embodiments, the olefinic side comprises (CH₂)n, wherein n is any integer between 1 to 6. In some embodiments, n is 3 for a cross-link length of 8 atoms. In some embodiments, n is 2 for a cross-link length of 6 atoms. An exemplary GLP-1 analog comprising an olefinic cross-link is described herein as SEQ ID NO: 17. Suitable methods of forming such intramolecular bridges are described in the art. See, for example, Schafmeister et al., J. Am. Chem. Soc. 122: 5891-5892 (2000) and Walensky et al., Science 305: 1466-1470 (2004). In alternative embodiments, the analog comprises O-allyl Ser residues located on adjacent helical turns, which are bridged together via ruthenium-catalyzed ring closing metathesis. Such procedures of cross-linking are described in, for example, Blackwell et al., Angew, Chem., Int. Ed. 37: 3281-3284 (1998).

In specific aspects, use of the unnatural thio-dialanine amino acid, lanthionine, which has been widely adopted as a peptidomimetic of cystine, is used to cross-link one turn of the alpha helix. Suitable methods of lanthionine-based cyclization are known in the art. See, for instance, Matteucci et al., Tetrahedron Letters 45: 1399-1401 (2004); Mayer et al., J. Peptide Res. 51: 432-436 (1998); Polinsky et al., J. Med. Chem. 35: 4185-4194 (1992); Osapay et al., J. Med. Chem. 40: 2241-2251 (1997); Fukase et al., Bull. Chem. Soc. Jpn. 65: 2227-2240 (1992); Harpp et al., J. Org. Chem. 36: 73-80 (1971); Goodman and Shao, Pure Appl. Chem. 68: 1303-1308 (1996); and Osapay and Goodman, J. Chem. Soc. Chem. Commun. 1599-1600 (1993).

In some embodiments, α,ω-diaminoalkane tethers, e.g., 1,4-diaminopropane and 1,5-diaminopentane) between two Glu residues at positions i and i+7 are used to stabilize the alpha helix of the analog. Such tethers lead to the formation of a bridge 9-atoms or more in length, depending on the length of the diaminoalkane tether. Suitable methods of producing peptides cross-linked with such tethers are described in the art. See, for example, Phelan et al., J. Am. Chem. Soc. 119: 455-460 (1997).

In yet other embodiments, a disulfide bridge is used to cross-link one or two turns of the alpha helix of the analog. Alternatively, a modified disulfide bridge in which one or both sulfur atoms are replaced by a methylene group resulting in an isosteric macrocyclization is used to stabilize the alpha helix of the analog. Suitable methods of modifying peptides with disulfide bridges or sulfur-based cyclization are described in, for example, Jackson et al., J. Am. Chem. Soc. 113: 9391-9392 (1991) and Rudinger and Jost, Experientia 20: 570-571 (1964).

In yet other embodiments, the alpha helix of the analog is stabilized via the binding of metal atom by two His residues or a His and Cys pair positioned at j and j+3, or i and i+4. The metal atom can be, for example, Ru(III), Cu(II), Zn(II), or Cd(II). Such methods of metal binding-based alpha helix stabilization are known in the art. See, for example, Andrews and Tabor, Tetrahedron 55: 11711-11743 (1999); Ghadiri et al., J. Am. Chem. Soc. 112: 1630-1632 (1990); and Ghadiri et al., J. Am. Chem. Soc. 119: 9063-9064 (1997).

The alpha helix of the analog can alternatively be stabilized through other means of peptide cyclizing, which means are reviewed in Davies, J. Peptide. Sci. 9: 471-501 (2003). The alpha helix can be stabilized via the formation of an amide bridge, thioether bridge, thioester bridge, urea bridge, carbamate bridge, sulfonamide bridge, and the like. For example, a thioester bridge can be formed between the C-terminus and the side chain of a Cys residue. Alternatively, a thioester can be formed via side chains of amino acids having a thiol (Cys) and a carboxylic acid (e.g., Asp, Glu). In another method, a cross-linking agent, such as a dicarboxylic acid, e.g., suberic acid (octanedioic acid), etc. can introduce a link between two functional groups of an amino acid side chain, such as a free amino, hydroxyl, thiol group, and combinations thereof.

DPP-IV Resistant Peptides

In some embodiments, the GLP-1 analog comprises at position 1 or 2, or at both positions 1 and 2, an amino acid which achieves resistance of the GLP-1 analog to dipeptidyl peptidase IV (DPP IV) cleavage. In some embodiments, the GLP-1 analog comprises an amino acid at position 1 that provides resistance to dipeptidyl peptidase IV (DPP IV) cleavage, wherein the amino acid is selected from the group consisting of: D-histidine, desaminohistidine, hydroxyl-histidine, acetyl-histidine, homo-histidine, N-methyl histidine, alpha-methyl histidine, imidazole acetic acid, or alpha, alpha-dimethyl imidiazole acetic acid (DMIA). In some embodiments, the GLP-1 analog comprises an amino acid at position 2 that provides resistance to dipeptidyl peptidase IV (DPP IV) cleavage, wherein the amino acid is selected from the group consisting of: D-serine, D-alanine, valine, glycine, N-methyl serine, N-methyl alanine, or alpha, aminoisobutyric acid. In some embodiments, the GLP-1 analog comprises at position 2 an amino acid which achieves resistance of the GLP-1 analog to DPP IV and the amino acid which achieves resistance of the GLP-1 analog to DPP IV is not D-serine.

In some aspects, the GLP-1 analog comprising an amino acid which achieves resistance of the GLP-1 analog to DPP IV further comprises an amino acid modification which stabilizes the alpha helix found in the C-terminal portion of GLP-1, e.g., through a covalent bond between amino acids at positions “i” and “i+4”, e.g., 12 and 16, 16 and 20, or 20 and 24. In some embodiments, this covalent bond is a lactam bridge between a glutamic acid at position 16 and a lysine at position 20. In some embodiments, this covalent bond is an intramolecular bridge other than a lactam bridge. For example, suitable covalent bonding methods include any one or more of olefin metathesis, lanthionine-based cyclization, disulfide bridge or modified sulfur-containing bridge formation, the use of α, ω-diaminoalkane tethers, the formation of metal-atom bridges, and other means of peptide cyclization.

Modification of Position 1

In some specific embodiments, the GLP-1 analog comprises (a) an amino acid substitution of His at position 1 with a large, aromatic amino acid and (b) an intramolecular bridge that stabilizes that alpha-helix in the C-terminal portion of the molecule (e.g., around positions 12-29). In specific embodiments, the amino acid at position 1 is replaced with Tyr, Phe, Trp, amino-Phe, nitro-Phe, chloro-Phe, sulfo-Phe, 4-pyridyl-Ala, methyl-Tyr, or 3-amino Tyr. The intramolecular bridge, in some embodiments, is any of those described herein. In some aspects, the intramolecular bridge is between the side chains of two amino acids that are separated by three intervening amino acids, i.e., between the side chains of amino acids i and i+4. In some embodiments, the intramolecular bridge is a lactam bridge. In some embodiments, the GLP-1 analog comprises a large, aromatic amino acid at position 1 and a lactam bridge between the amino acids at positions 16 and 20 of the analog. Such a GLP-1 analog in some aspects further comprises one or more (e.g., two, three, four, five or more) of the other modifications described herein. For example, the GLP-1 analog can comprise an amide in place of the C-terminal carboxylate. Also, in some embodiments, such GLP-1 analogs further comprise one or more of a large aliphatic amino acid at position 17, an imidazole containing amino acid at position 18, and a positive-charged amino acid at position 19. In some embodiments, the GLP-1 analogs comprising a modification at position 1 and an intramolecular bridge further comprises the amino acid sequence Ile-His-Gln at positions 17-19. Such modifications can be made without destroying activity of the GLP-1 analog at the GLP-1 receptor and the glucagon receptor. In some embodiments, the GLP-1 analog additionally comprises an acylated or alkylated amino acid residue.

Modification of Position 3

In some embodiments, the third amino acid of a modified SEQ ID NO: 44 or SEQ ID NO: 38 is an acidic, basic, or hydrophobic amino acid residue. In some embodiments, the acidic, basic, or hydrophobic amino acid is glutamic acid, ornithine, norleucine. In some embodiments, the amino acid at position 3 of the GLP-1 analog is a naturally occurring or a non-naturally occurring or non-coded amino acid comprising a side chain of Structure I, II or III:

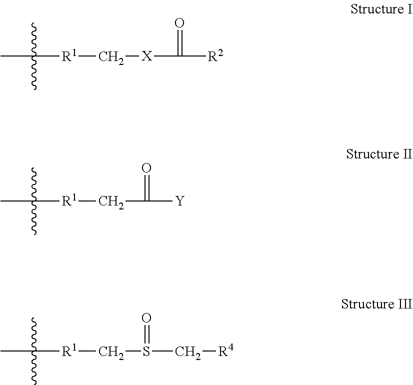

wherein R¹ is C₀₋₃ alkyl or C₀₋₃ heteroalkyl; R² is NHR⁴ or C₁₋₃ alkyl; R³ is C₁₋₃ alkyl; R⁴ is H or C₁₋₃ alkyl; X is NH, O, or S; and Y is NHR⁴, SW, or OR³. In some embodiments, X is NH or Y is NHR⁴. In some embodiments, R¹ is C₀₋₂ alkyl or C₁ heteroalkyl. In some embodiments, R² is NHR⁴ or C₁ alkyl. In some embodiments, R⁴ is H or C¹ alkyl. In exemplary embodiments, an amino acid comprising a side chain of Structure I is provided where, R¹ is CH₂—S, X is NH, and R² is CH₃ (acetamidomethyl-cysteine, C(Acm)); R¹ is CH₂, X is NH, and R² is CH₃ (acetyldiaminobutanoic acid, Dab(Ac)); R¹ is C₀ alkyl, X is NH, R² is NHR⁴, and R⁴ is H (carbamoyldiaminopropanoic acid, Dap(urea)); or R¹ is CH₂—CH₂, X is NH, and R² is CH₃ (acetylornithine, Orn(Ac)). In exemplary embodiments, an amino acid comprising a side chain of Structure II is provide where, R¹ is CH₂, Y is NHR⁴, and R⁴ is CH₃ (methylglutamine, Q(Me)); In exemplary embodiments, an amino acid comprising a side chain of Structure III is provided where, R¹ is CH₂ and R⁴ is H (methionine-sulfoxide, M(O)); In specific embodiments, the amino acid at position 3 is substituted with Dab(Ac) The GLP-1 agonist peptides disclosed herein can be further modified to comprise at least three alpha helix promoting amino acids, modified to comprise (i) an acylated or alkylated amino acid at position 10, (ii) an alpha helix promoting amino acid at position 16, (iii) an aliphatic amino acid at position 17 and/or 18, and (iv) at least one charged amino acid located C-terminal to position 27, and, optionally, further modifications; modified to comprise at least three amino acids of the amino acids 18-24 of Exendin-4 (SEQ ID NO: 29) at the corresponding positions of the GLP-1 analog.

Modification of Position 15

In some embodiments, the GLP-1 analogs comprise a modified SEQ ID NO: 44 or SEQ ID NO: 38 with an amino acid modification at position 15 which improves stability. In some aspects, the amino acid at position 15 of SEQ ID NO: 44 or SEQ ID NO: 38 is deleted or substituted with glutamic acid, homoglutamic acid, cysteic acid or homocysteic acid. Such modifications reduce degradation or cleavage of the analog over time, especially in acidic or alkaline buffers, e.g., buffers at a pH within the range of 5.5 to 8. In some embodiments, the GLP-1 analogs comprising this modification retains at least 75%, 80%, 90%, 95%, 96%, 97%, 98% or 99% of the original analog after 24 hours at 25° C.

Modification of Position 16

In accordance with one embodiment, analogs of GLP-1 are provided that have enhanced potency and optionally improved solubility and stability. In one embodiment, enhanced glucagon and GLP-1 potency is provided by an amino acid modification at position 16 of SEQ ID NO: 44 or SEQ ID NO: 38. By way of nonlimiting example, such enhanced potency can be provided by substituting the naturally occurring amino acid at position 16 with glutamic acid or with another negative-charged amino acid having a side chain with a length of 4 atoms, or alternatively with any one of glutamine, homoglutamic acid, or homocysteic acid, or a charged amino acid having a side chain containing at least one heteroatom, (e.g., N, O, S, P) and with a side chain length of about 4 (or 3-5) atoms. In some embodiments, the GLP-1 analog comprises a modified SEQ ID NO: 44 or SEQ ID NO: 38 comprising a substitution of the Gly at position 16 with an amino acid selected from the group consisting of glutamic acid, glutamine, homoglutamic acid, homocysteic acid, or threonine. In some aspects, the serine residue at position 16 is substituted with an amino acid selected from the group consisting of glutamic acid, glutamine, homoglutamic acid and homocysteic acid. In some specific aspects, the residue at position 16 is glutamic acid or a conservative substitution thereof (e.g. an Exendin-4 amino acid).

In alternative embodiments, the GLP-1 analog comprises a modified SEQ ID NO: 44 or SEQ ID NO: 38 modified by a substitution at position 16 with Thr or Aib or another alpha helix promoting amino acid as described above. In some embodiments, the alpha helix promoting amino acid forms a non-covalent intramolecular bridge with an amino acid at j+3 or i+4.

Modification at Positions 17-18

In some embodiments, the GLP-1 analog comprises a modified SEQ ID NO: 44 or SEQ ID NO: 38 in which the amino acid at position 17 is substituted with another amino acid as described herein, e.g., Gln, an amino acid comprising a hydrophilic moiety, an alpha helix promoting amino acid. In some embodiments, the alpha helix promoting amino acid forms a non-covalent intramolecular bridge with an amino acid at j+3 or i+4. In some embodiments, the amino acid at position 18 is substituted with another amino acid as described herein. In exemplary aspects, the amino acid at position 18 is an alpha, alpha, disubstituted amino acid, e.g., Aib. In some aspects, the amino acid at position 18 is a small aliphatic amino acid, e.g., Ala. In some specific aspects, the amino acid at position 18 is a small aliphatic amino acid, e.g., Ala, and the Arg at position 17 remains unmodified.

Modification of Position 20

Enhanced activity at the GLP-1 receptor is also provided by an amino acid modification at position 20. In some embodiments, the amino acid at position 20 is replaced with an alpha helix promoting amino acid, e.g. Aib, as described above. In some embodiments, the alpha helix promoting amino acid forms a non-covalent intramolecular bridge with an amino acid at j-3 or i-4. In some specific embodiments the amino acid is a hydrophilic amino acid having a side chain that is either charged or has an ability to hydrogen-bond, and is at least about 5 (or about 4-6) atoms in length, for example, lysine, citrulline, arginine, or ornithine, and optionally forms a salt bridge with another alpha helix promoting amino acid at position 16, e.g. a negative charged amino acid. Such modifications in some particular aspects reduce degradation that occurs through deamidation of Gln and in some embodiments, increase the activity of the GLP-1 analog at the GLP-1 receptor. In some aspects, the amino acid at position 20 is Glu or Lys or Aib.

Modification at Positions 21, 23, 24, and 28

In some embodiments, position 21 and/or position 24 is modified by substitution with an alpha helix promoting amino acid. In some embodiments, the alpha helix promoting amino acid forms a non-covalent intramolecular bridge with an amino acid at j-3 or i-4. In some aspects, the alpha helix promoting amino acid is Aib.

In exemplary embodiments, the amino acid at position 23 is an Ile.

In exemplary aspects, the amino acid at position 28 is an alpha, alpha, disubstituted amino acid, e.g., Aib.

Charged C-Terminus

In some embodiments, the GLP-1 analog is modified by amino acid substitutions and/or additions that introduce a charged amino acid into the C-terminal portion of the analog. In some embodiments, such modifications enhance stability and solubility. As used herein the term “charged amino acid” or “charged residue” refers to an amino acid that comprises a side chain that is negative-charged (i.e., de-protonated) or positive-charged (i.e., protonated) in aqueous solution at physiological pH. In some aspects, these amino acid substitutions and/or additions that introduce a charged amino acid modifications are at a position C-terminal to position 27 of SEQ ID NO: 44 or SEQ ID NO: 38. In some embodiments, one, two or three (and in some instances, more than three) charged amino acids are introduced within the C-terminal portion (e.g., position(s)C-terminal to position 27). In accordance with some embodiments, the native amino acid(s) at positions 28 and/or 29 are substituted with a charged amino acids, and/or in a further embodiment one to three charged amino acids are also added to the C-terminus of the analog. In exemplary embodiments, one, two or all of the charged amino acids are negative-charged. The negative-charged amino acid in some embodiments is aspartic acid, glutamic acid, cysteic acid, homocysteic acid, or homoglutamic acid. In some aspects, these modifications increase solubility, e.g., provide at least 2-fold, 5-fold, 10-fold, 15-fold, 25-fold, 30-fold or greater solubility relative to native GLP-1 at a given pH between about 5.5 and 8, e.g., pH 7, when measured after 24 hours at 25° C.

C-Terminal Truncation

In accordance with some embodiments, the GLP-1 analogs disclosed herein are modified by truncation of the C-terminus by one or two amino acid residues. Such modified GLP-1 peptides, as shown herein, retain similar activity and potency at the glucagon receptor and GLP-1 receptor. In this regard, the GLP-1 peptides can comprise amino acids 1-27 or 1-28 of the GLP-1 analog (SEQ ID NO: 38), optionally with any of the additional modifications described herein.

Charge-Neutral C-Terminus

In some embodiments, the GLP-1 analog comprises a modified SEQ ID NO: 44 or SEQ ID NO: 38 in which the carboxylic acid of the C-terminal amino acid is replaced with a charge-neutral group, such as an amide or ester. Without being bound to any particular theory, such modifications in certain aspects increases activity of the glucagon analog at the GLP-1 receptor. Accordingly, in some embodiments, the GLP-1 analog is an amidated peptide, such that the C-terminal residue comprises an amide in place of the alpha carboxylate of an amino acid. As used herein a general reference to a peptide or analog is intended to encompass peptides that have a modified amino terminus, carboxy terminus, or both amino and carboxy termini. For example, an amino acid chain composing an amide group in place of the terminal carboxylic acid is intended to be encompassed by an amino acid sequence designating the standard amino acids.

In some embodiments, the GLP-1 agonist comprises the sequence of native glucagon (SEQ ID NO: 1) modified by one or more of the following amino acid modifications:
(i) Substitution of Ser at position 2 with Ala;
(ii) Substitution of Tyr at position 10 with Val or Phe, or Trp;
(iii) Substitution of Lys at position 12 with Arg;
(iv) Substitution of Arg at position 17 with Gln or a small aliphatic amino acid, e.g., Ala, or a large aliphatic amino acid, e.g., Be;
(v) Substitution of Arg at position 18 with a small aliphatic amino acid, e.g., Ala; or an imidazole-containing amino acid, e.g., His;

(vi) Substitution of Ala at position 19 with a positive-charged amino acid, e.g., Gln;
(vii) Substitution of Val at position 23 with Ile,
(viii) Substitution of Thr at position 29 with Gly or Gln, and
(ix) Substitution of methionine at position 27 with leucine or norleucine.

In some embodiments, the glucagon analogs described herein are glycosylated, amidated, carboxylated, phosphorylated, esterified, N-acylated, cyclized via, e.g., a disulfide bridge, or converted into a salt (e.g., an acid addition salt, a basic addition salt), and/or optionally dimerized, multimerized, or polymerized, or conjugated.

Any of the modifications described herein, including, for example, the modifications which increase or decrease glucagon receptor activity and which increase GLP-1 receptor activity, can be applied individually or in combination. Combinations of the modifications that increase GLP-1 receptor activity may provide higher GLP-1 activity than any of such modifications taken alone.

### Exemplary Embodiments

In exemplary aspects, the peptides of the present disclosures exhibit activity at the GLP-1 receptor which is greater than that of native GLP-1. Accordingly, in exemplary aspects, the peptides of the present disclosures exhibit greater than or about 100% of the activity of native-GLP-1 at the GLP-1 receptor. In exemplary aspects, the peptides of the present disclosures exhibit greater than or about 150%, greater than or about 200%, greater than or about 250%, greater than or about 300%, greater than or about 350%, greater than or about 400%, greater than or about 450%, greater than or about 500%, greater than or about 550%, greater than or about 600%, greater than or about 650%, greater than or about 700%, greater than or about 750%, greater than or about 800%, greater than or about 850%, greater than or about 900%, greater than or about 950%, or greater than or about 1000% of the activity of native-GLP-1 at the GLP-1 receptor.

In exemplary embodiments, the GLP-1 agonist peptide comprises the amino acid sequence of SEQ ID NO: 30.

In exemplary embodiments, the GLP-1 agonist peptide comprises the amino acid sequence of SEQ ID NO: 31.

In exemplary embodiments, the GLP-1 agonist peptide comprises the amino acid sequence of SEQ ID NO: 32.

In exemplary embodiments, the GLP-1 agonist peptide comprises the amino acid sequence of SEQ ID NO: 33.

In exemplary embodiments, the GLP-1 agonist peptide comprises the amino acid sequence of SEQ ID NO: 34 and exhibits at least 100-fold selectivity for the human GLP-1 receptor versus the GIP receptor.

In exemplary embodiments, the GLP-1 agonist peptide comprises the amino acid sequence of SEQ ID NO: 35.

In one embodiment the GLP-1 analog comprises the sequence

(SEQ ID NO: 43)
X₁X₂X₃GTFTSDVSX₁₂YLX₁₅X₁₆QAAX₂₀X₂₁FIX₂₄WLX₂₇X₂₈X₂₉

wherein

X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);

X₂ is selected from the group consisting of Ser, D-Ser, Ala, D-Ala, Gly, N-methyl-Ser, Aib, Val, or α-amino-N-butyric acid;

X₃ is glutamic acid, ornithine, norleucine;

X₁₂ is Lys or Arg;

X₁₅ is Asp, Glu, cysteic acid, homoglutamic acid or homocysteic acid;

X₁₆ is Aib, glutamic acid, glutamine, homoglutamic acid, homocysteic acid, threonine or glycine;

X₂₀ is Glu, Lys or Aib;

X₂₁ is Glu or Aib;

X₂₄ is Ala, Glu, Lys or Aib;

X₂₇ is Met, Val, Leu or Nle;

X₂₈ is Glu, Lys or Aib;

X₂₉ is Gly, Gln, Asp or Glu. In a further embodiment the peptide of SEQ ID NO: 43 is further modified to enhance the stability of the C-terminal alpha helix. In one embodiment the C-terminal alpha helix is stabilized by

i) an intramolecular bridge is formed between the side chains of amino acids at positions i and i+4, wherein i is 12, 16, 20 or 24;

ii) an Aib is present at 1, 2, 3 or 4 of positions 16, 20, 21 and 24; or

iii) both i) and ii). In one embodiment Aib is present at position 16.

In one embodiment the GLP-1 analog comprises the sequence

(SEQ ID NO: 37)
X₁X₂EGTFTSDVSSYLX₁₅X₁₆QAAX₂₀X₂₁FIX₂₄WLX₂₇KG-Z

wherein

X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);

X₂ is selected from the group consisting of Ser, D-Ser, Ala, D-Ala, Gly, N-methyl-Ser, Aib, Val, or α-amino-N-butyric acid;

X₁₅ is Asp, Glu, cysteic acid, homoglutamic acid or homocysteic acid;

X₁₆ is Gly, Glu, Lys or Aib,

X₂₀ is Glu, Ala, Lys or Aib;

X₂₁ is Glu or Aib;

X₂₄ is Ala, Glu, Lys or Aib;

X₂₇ is Met, Leu, Val or Nle; and

Z is selected from the group consisting of —COOH, —C(O)NH₂-Arg-COOH, Arg-Gly-COOH, X₄₀PSSGAPPPSX₄₀ (SEQ ID NO: 36) and GPSSGAPPPSX₄₀, (SEQ ID NO: 26) wherein X₄₀ is an amino acid selected from the group consisting of Cys or Lys. In accordance with one embodiment X₁₅ is Glu, X₁₆ is Gly or Aib, X₂₀ is Lys or Aib, X₂₁ is Glu and X₂₄ is Ala or Aib. In one embodiment the GLP-1 analog is a peptide comprising a modified SEQ ID NO: 44 or SEQ ID NO: 38, wherein X₁₆ is Aib. In one embodiment the GLP-1 analog is a peptide comprising a modified SEQ ID NO: 38 wherein X₁ is His, X₂₀ is Lys; and X₂₄ is Ala. In a further embodiment X₂ is D-serine or Aib and X₁₆ is Aib. In one embodiment the GLP-1 agonist comprises a peptide selected from the group consisting of

(SEQ ID NO: 45)
HX₂EGTFTSDVSSYLEX₁₆QAAKEFIAWLVKG-Z
(SEQ ID NO: 38)
HAEGTFTSDVSSYLEGQAAKEFIAWLVKG-Z
or
(SEQ ID NO: 39)
HAEGTFTSDVSSYLE(Aib)QAAKEFIAWLVKG-Z

wherein

X₂ is D-serine or Aib;

X₁₆ is Gly, Glu or Aib; and

Z is selected from the group consisting of —COOH, —C(O)NH₂-Arg-COOH, Arg-Gly-COOH, X₄₀PSSGAPPPSX₄₀ (SEQ ID NO: 36), GPSSGAPPPS (SEQ ID NO: 29) and GPSSGAPPPSX₄₀ (SEQ ID NO: 26), wherein X₄₀ is an amino acid selected from the group consisting of Cys or Lys. In one embodiment the GLP-1 agonist comprises a peptide selected from the group consisting of

(SEQ ID NO: 40)
HAEGTFTSDVSSYLEGQAAKEFIAWLVKGGPSSGAPPPSX₄₀
or
(SEQ ID NO: 41)
HAEGTFTSDVSSYLE(Aib)QAAKEFIAWLVKGGPSSGAPPPSX₄₀

wherein X₄₀ is Lys.

In accordance with one embodiment a conjugate having the general formula Q-L-Y is provided wherein

Y is selected from the group consisting of thyroxine T4 (3,5,3′,5′-tetra-iodothyronine), and 3,5,3′-triiodo L-thyronine; and

Q comprises the sequence HX₂EGTFTSDVSSYLEX₁₆QAAKEFIAWLVKG-Z (SEQ ID NO: 45), HAEGTFTSDVSSYLEGQAAKEFIAWLVKG-Z (SEQ ID NO: 38) or HAEGTFTSDVSSYLE(Aib)QAAKEFIAWLVKG-Z (SEQ ID NO: 39) wherein

X₂ is D-serine or Aib;

X₁₆ is Gly, Glu or Aib; and

Z is selected from the group consisting of —COOH, —C(O)NH₂-Arg-COOH, -Lys-COOH, Arg-Gly-COOH, X₄₀PSSGAPPPSX₄₀ (SEQ ID NO: 36) and GPSSGAPPPSX₄₀ (SEQ ID NO: 26), wherein X₄₀ is an amino acid selected from the group consisting of Cys or Lys. In one embodiment the thyroid hormone receptor ligand is covalently attached to the side chain amine of a Lys at position 24 or 29 or at position 30-41 of a C-terminal extension relative to native GLP-1, optionally at position 30 or 40. The thyroid hormone receptor ligand can be either directly linked to the GLP-1 agonist peptide (e.g., by linkage to a side chain of an amino acid of GLP-1) or through a linker. In one embodiment the linker is an amino acid or dipeptide linker, including for example a gamma glutamic acid (γGlu) spacer or a γGlu-γGlu spacer added to the carboxylate of the thyroid hormone receptor.

### Structure of L

In some embodiments, L is a bond. In these embodiments, Q and Y are conjugated together by reacting a nucleophilic reactive moiety on Q with and electrophilic reactive moiety on Y. In alternative embodiments, Q and Y are conjugated together by reacting an electrophilic reactive moiety on Q with a nucleophilic moiety on Y. In exemplary embodiments, L is an amide bond that forms upon reaction of an amine on Q (e.g. an ε-amine of a lysine residue) with a carboxyl group on Y. In alternative embodiments, Q and or Y are derivatized with a derivatizing agent before conjugation.

In some embodiments, L is a linking group. In some embodiments, L is a bifunctional linker and comprises only two reactive groups before conjugation to Q and Y. In embodiments where both Q and Y have electrophilic reactive groups, L comprises two of the same or two different nucleophilic groups (e.g. amine, hydroxyl, thiol) before conjugation to Q and Y. In embodiments where both Q and Y have nucleophilic reactive groups, L comprises two of the same or two different electrophilic groups (e.g. carboxyl group, activated form of a carboxyl group, compound with a leaving group) before conjugation to Q and Y. In embodiments where one of Q or Y has a nucleophilic reactive group and the other of Q or Y has an electrophilic reactive group, L comprises one nucleophilic reactive group and one electrophilic group before conjugation to Q and Y.

L can be any molecule with at least two reactive groups (before conjugation to Q and Y) capable of reacting with each of Q and Y. In some embodiments L has only two reactive groups and is bifunctional. L (before conjugation to the peptides) can be represented by Formula VI:

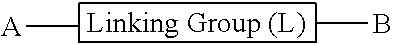

wherein A and B are independently nucleophilic or electrophilic reactive groups. In some embodiments A and B are either both nucleophilic groups or both electrophilic groups. In some embodiments one of A or B is a nucleophilic group and the other of A or B is an electrophilic group.

In some embodiments, L comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers may be selected according to their expected solubility (hydrophilicity) so as to provide a more soluble conjugate. In some embodiments, L provides a functional group that is subject to cleavage by an enzyme or other catalyst or hydrolytic conditions found in the target tissue or organ or cell. In some embodiments, the length of L is long enough to reduce the potential for steric hindrance.

In some embodiments, the linking group is hydrophilic such as, for example, polyalkylene glycol. Before conjugation to the peptides of the composition, the hydrophilic linking group comprises at least two reactive groups (A and B), as described herein and as shown below:

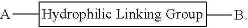

In specific embodiments, the linking group is polyethylene glycol (PEG). The PEG in certain embodiments has a molecular weight of about 100 Daltons to about 10,000 Daltons, e.g. about 500 Daltons to about 5000 Daltons. The PEG in some embodiments has a molecular weight of about 10,000 Daltons to about 40,000 Daltons.

In some embodiments, the hydrophilic linking group comprises either a maleimido or an iodoacetyl group and either a carboxylic acid or an activated carboxylic acid (e.g. NHS ester) as the reactive groups. In these embodiments, the maleimido or iodoacetyl group can be coupled to a thiol moiety on Q or Y and the carboxylic acid or activated carboxylic acid can be coupled to an amine on Q or Y with or without the use of a coupling reagent. Any appropriate coupling agent known to one skilled in the art can be used to couple the carboxylic acid with the amine. In some embodiments, the linking group is maleimido-PEG(20 kDa)-COOH, iodoacetyl-PEG(20 kDa)-COOH, maleimido-PEG(20 kDa)-NHS, or iodoacetyl-PEG(20 kDa)-NHS.

In some embodiments, the linking group is comprised of an amino acid, a dipeptide, a tripeptide, or a polypeptide, wherein the amino acid, dipeptide, tripeptide, or polypeptide comprises at least two activating groups, as described herein. In some embodiments, the linking group (L) comprises a moiety selected from the group consisting of: amino, ether, thioether, maleimido, disulfide, amide, ester, thioester, alkene, cycloalkene, alkyne, trizoyl, carbamate, carbonate, cathepsin B-cleavable, and hydrazone. In some embodiments, the linking group is an amino acid selected from the group Asp, Glu, homoglutamic acid, homocysteic acid, cysteic acid, gamma-glutamic acid. In some embodiments, the linking group is a dipeptide selected from the group consisting of: Ala-Ala, β-Ala- β-Ala, Leu-Leu, Pro-Pro, γ-aminobutyric acid- γ-aminobutyric acid, and γ-Glu- γ-Glu. In one embodiment L comprises gamma-glutamic acid.

In embodiments where Q and Y are conjugated together by reacting a carboxylic acid with an amine, an activating agent can be used to form an activated ester of the carboxylic acid. The activated ester of the carboxylic acid can be, for example, N-hydroxysuccinimide (NHS), tosylate (Tos), mesylate, triflate, a carbodiimide, or a hexafluorophosphate. In some embodiments, the carbodiimide is 1,3-dicyclohexylcarbodiimide (DCC), 1,1′-carbonyldiimidazole (CDI), 1-ethyl-3-(3-dimethylaminopropyl)carbodiimide hydrochloride (EDC), or 1,3-diisopropylcarbodiimide (DICD). In some embodiments, the hexafluorophosphate is selected from a group consisting of hexafluorophosphate benzotriazol-1-yl-oxy-tris(dimethylamino)phosphonium hexafluorophosphate (BOP), benzotriazol-1-yl-oxytripyrrolidinophosphonium hexafluorophosphate (PyBOP), 2-(1H-7-azabenzotriazol-1-yl)-1,1,3,3-tetramethyl uronium hexafluorophosphate (HATU), and o-benzotriazole-N,N,N′,N′-tetramethyl-uronium-hexafluoro-phosphate (HBTU).

In some embodiments, Q comprises a nucleophilic reactive group (e.g. the amino group, thiol group, or hydroxyl group of the side chain of lysine, cysteine or serine) that is capable of conjugating to an electrophilic reactive group on Y or L. In some embodiments, Q comprises an electrophilic reactive group (e.g. the carboxylate group of the side chain of Asp or Glu) that is capable of conjugating to a nucleophilic reactive group on Y or L. In some embodiments, Q is chemically modified to comprise a reactive group that is capable of conjugating directly to Y or to L. In some embodiments, Q is modified at the C-terminal to comprise a natural or nonnatural amino acid with a nucleophilic side chain, such as an amino acid represented by Formula I, Formula II, or Formula III, as previously described herein (see Acylation and alkylation). In exemplary embodiments, the C-terminal amino acid of Q is selected from the group consisting of lysine, ornithine, serine, cysteine, and homocysteine. For example, the C-terminal amino acid of Q can be modified to comprise a lysine residue. In some embodiments, Q is modified at the C-terminal amino acid to comprise a natural or nonnatural amino acid with an electrophilic side chain such as, for example, Asp and Glu. In some embodiments, an internal amino acid of Q is substituted with a natural or nonnatural amino acid having a nucleophilic side chain, such as an amino acid represented by Formula I, Formula II, or Formula III, as previously described herein (see Acylation and alkylation). In exemplary embodiments, the internal amino acid of Q that is substituted is selected from the group consisting of lysine, ornithine, serine, cysteine, and homocysteine. For example, an internal amino acid of Q can be substituted with a lysine residue. In some embodiments, an internal amino acid of Q is substituted with a natural or nonnatural amino acid with an electrophilic side chain, such as, for example, Asp and Glu.

In some embodiments, Y comprises a reactive group that is capable of conjugating directly to Q or to L. In some embodiments, Y comprises a nucleophilic reactive group (e.g. amine, thiol, hydroxyl) that is capable of conjugating to an electrophilic reactive group on Q or L. In some embodiments, Y comprises electrophilic reactive group (e.g. carboxyl group, activated form of a carboxyl group, compound with a leaving group) that is capable of conjugating to a nucleophilic reactive group on Q or L.

### Stability of L In Vivo

In some embodiments, L is stable in vivo. In some embodiments, L is stable in blood serum for at least 5 minutes, e.g. less than 25%, 20%, 15%, 10% or 5% of the conjugate is cleaved when incubated in serum for a period of 5 minutes. In other embodiments, L is stable in blood serum for at least 10, or 20, or 25, or 30, or 60, or 90, or 120 minutes, or 3, 4, 5, 6, 7, 8, 9, 10, 12, 15, 18 or 24 hours. In these embodiments, L does not comprise a functional group that is capable of undergoing hydrolysis in vivo. In some exemplary embodiments, L is stable in blood serum for at least about 72 hours. Nonlimiting examples of functional groups that are not capable of undergoing significant hydrolysis in vivo include amides, ethers, and thioethers. For example, the following compound is not capable of undergoing significant hydrolysis in vivo:

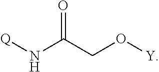

In some embodiments, L is hydrolyzable in vivo. In these embodiments, L comprises a functional group that is capable of undergoing hydrolysis in vivo. Nonlimiting examples of functional groups that are capable of undergoing hydrolysis in vivo include esters, anhydrides, and thioesters. For example the following compound is capable of undergoing hydrolysis in vivo because it comprises an ester group:

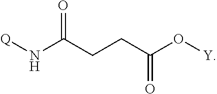

In some exemplary embodiments L is labile and undergoes substantial hydrolysis within 3 hours in blood plasma at 37° C., with complete hydrolysis within 6 hours. In some exemplary embodiments, L is not labile.

In some embodiments, L is metastable in vivo. In these embodiments, L comprises a functional group that is capable of being chemically or enzymatically cleaved in vivo (e.g., an acid-labile, reduction-labile, or enzyme-labile functional group), optionally over a period of time. In these embodiments, L can comprise, for example, a hydrazone moiety, a disulfide moiety, or a cathepsin-cleavable moiety. When L is metastable, and without intending to be bound by any particular theory, the Q-L-Y conjugate is stable in an extracellular environment, e.g., stable in blood serum for the time periods described above, but labile in the intracellular environment or conditions that mimic the intracellular environment, so that it cleaves upon entry into a cell. In some embodiments when L is metastable, L is stable in blood serum for at least about 24, 25, 26, 27, 28, 29, 30, 31, 32, 33, 34, 35, 36, 42, or 48 hours, for example, at least about 48, 54, 60, 66, or 72 hours, or about 24-48, 48-72, 24-60, 36-48, 36-72, or 48-72 hours.

In accordance with one embodiment L-Y comprises the structure:

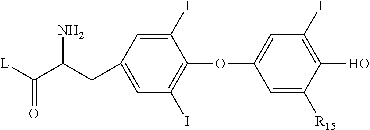

wherein

L is a bond, an amino acid, or dipeptide joining Q to Y; and

R₁₅ is H or I. In one embodiment L is γ-Glu or the dipeptide, γ-Glu- γ-Glu. In one embodiment L-Y comprises the structure

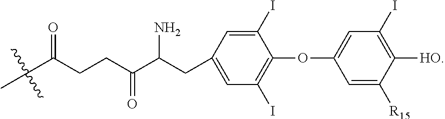

The GLP-1 agonists of the present invention can be further modified to improve the peptide's solubility and stability in aqueous solutions while retaining the biological activity of the peptide. In accordance with some embodiments, introduction of hydrophilic groups at one or more positions selected from positions 16, 17, 20, 21, 24 and 29 of the peptide of SEQ ID NO: 44 or SEQ ID NO: 38, or a GLP-1 agonist analog thereof, are anticipated to improve the solubility and stability of the GLP-1 agonist.

In accordance with some embodiments the pegylated GLP-1 agonist comprises two or more polyethylene glycol chains covalently bound to the peptide wherein the total molecular weight of the polyethylene glycol chains is about 1,000 to about 5,000 Daltons. In some embodiments the pegylated GLP-1 agonist comprises a PEG chain covalently linked to the amino acid residue at position 21 and at position 24, and wherein the combined molecular weight of the two PEG chains is about 1,000 to about 5,000 Daltons. In another embodiment the pegylated GLP-1 agonist comprises a PEG chain covalently linked to the amino acid residue at position 21 and at position 24, and wherein the combined molecular weight of the two PEG chains is about 5,000 to about 20,000 Daltons.

The polyethylene glycol chain may be in the form of a straight chain or it may be branched. In accordance with some embodiments the polyethylene glycol chain has an average molecular weight selected from the range of about 500 to about 40,000 Daltons. In some embodiments the polyethylene glycol chain has a molecular weight selected from the range of about 500 to about 5,000 Daltons. In another embodiment the polyethylene glycol chain has a molecular weight of about 20,000 to about 40,000 Daltons.

Any of the GLP-1 agonist peptides described above may be further modified to include a covalent or non-covalent intramolecular bridge or an alpha helix-stabilizing amino acid within the C-terminal portion of the GLP-1 peptide (amino acid positions 12-29). In accordance with some embodiments, the GLP-1 peptide comprises any one or more of the modifications discussed above in addition to an amino acid substitution at positions 16, 20, 21, or 24 (or a combination thereof) with an α,α-disubstituted amino acid, e.g., Aib. In accordance with another embodiment, the GLP-1 peptide comprises any one or more modifications discussed above in addition to an intramolecular bridge, e.g., a lactam, between the side chains of the amino acids at positions 16 and 20 of the GLP-1 peptide.

### Fc Fusion Heterologous Moiety

In some embodiments Q is conjugated, e.g., fused to an immunoglobulin or portion thereof (e.g. variable region, CDR, or Fc region). Known types of immunoglobulins (Ig) include IgG, IgA, IgE, IgD or IgM. The Fc region is a C-terminal region of an Ig heavy chain, which is responsible for binding to Fc receptors that carry out activities such as recycling (which results in prolonged half-life), antibody dependent cell-mediated cytotoxicity (ADCC), and complement dependent cytotoxicity (CDC).

For example, according to some definitions the human IgG heavy chain Fc region stretches from Cys226 to the C-terminus of the heavy chain. The “hinge region” generally extends from Glu216 to Pro230 of human IgG1 (hinge regions of other IgG isotypes may be aligned with the IgG1 sequence by aligning the cysteines involved in cysteine bonding). The Fc region of an IgG includes two constant domains, CH2 and CH3. The CH2 domain of a human IgG Fc region usually extends from amino acids 231 to amino acid 341. The CH3 domain of a human IgG Fc region usually extends from amino acids 342 to 447. References made to amino acid numbering of immunoglobulins or immunoglobulin fragments, or regions, are all based on Kabat et al. 1991, Sequences of Proteins of Immunological Interest, U.S. Department of Public Health, Bethesda, Md. In a related embodiment, the Fc region may comprise one or more native or modified constant regions from an immunoglobulin heavy chain, other than CHL for example, the CH2 and CH3 regions of IgG and IgA, or the CH3 and CH4 regions of IgE.

Suitable conjugate moieties include portions of immunoglobulin sequence that include the FcRn binding site. FcRn, a salvage receptor, is responsible for recycling immunoglobulins and returning them to circulation in blood. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The major contact sites include amino acid residues 248, 250-257, 272, 285, 288, 290-291, 308-311, and 314 of the CH2 domain and amino acid residues 385-387, 428, and 433-436 of the CH3 domain.

Some conjugate moieties may or may not include FcγR binding site(s). FcγR are responsible for ADCC and CDC. Examples of positions within the Fc region that make a direct contact with FcγR are amino acids 234-239 (lower hinge region), amino acids 265-269 (B/C loop), amino acids 297-299 (C′/E loop), and amino acids 327-332 (F/G) loop (Sondermann et al., Nature 406: 267-273, 2000). The lower hinge region of IgE has also been implicated in the FcRI binding (Henry, et al., Biochemistry 36, 15568-15578, 1997). Residues involved in IgA receptor binding are described in Lewis et al., (J Immunol. 175:6694-701, 2005). Amino acid residues involved in IgE receptor binding are described in Sayers et al. (J Biol Chem. 279(34):35320-5, 2004).

Amino acid modifications may be made to the Fc region of an immunoglobulin. Such variant Fc regions comprise at least one amino acid modification in the CH3 domain of the Fc region (residues 342-447) and/or at least one amino acid modification in the CH2 domain of the Fc region (residues 231-341). Mutations believed to impart an increased affinity for FcRn include T256A, T307A, E380A, and N434A (Shields et al. 2001, J. Biol. Chem. 276:6591). Other mutations may reduce binding of the Fc region to FcγRI, FcγRIIA, FcγRIIB, and/or FcγRIIIA without significantly reducing affinity for FcRn. For example, substitution of the Asn at position 297 of the Fc region with Ala or another amino acid removes a highly conserved N-glycosylation site and may result in reduced immunogenicity with concomitant prolonged half-life of the Fc region, as well as reduced binding to FcγRs (Routledge et al. 1995, Transplantation 60:847; Friend et al. 1999, Transplantation 68:1632; Shields et al. 1995, J. Biol. Chem. 276:6591). Amino acid modifications at positions 233-236 of IgG1 have been made that reduce binding to FcγRs (Ward and Ghetie 1995, Therapeutic Immunology 2:77 and Armour et al. 1999, Eur. J. Immunol. 29:2613). Some exemplary amino acid substitutions are described in U.S. Pat. Nos. 7,355,008 and 7,381,408, each incorporated by reference herein in its entirety.

### Hydrophilic Heterologous Moiety

In some embodiments, Q described herein is covalently bonded to a hydrophilic moiety. Hydrophilic moieties can be attached to Q under any suitable conditions used to react a protein with an activated polymer molecule. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., alpha-iodo acetic acid, alpha-bromoacetic acid, alpha-chloroacetic acid). If attached to the peptide by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995).

Further activating groups which can be used to link the hydrophilic moiety (water soluble polymer) to a protein include an alpha-halogenated acyl group (e.g., alpha-iodo acetic acid, alpha-bromoacetic acid, alpha-chloroacetic acid). In specific aspects, an amino acid residue of the peptide having a thiol is modified with a hydrophilic moiety such as PEG. In some embodiments, an amino acid on Q comprising a thiol is modified with maleimide-activated PEG in a Michael addition reaction to result in a PEGylated peptide comprising the thioether linkage shown below:

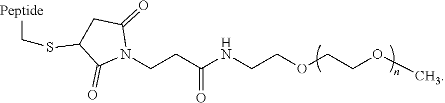

In some embodiments, the thiol of an amino acid of Q is modified with a haloacetyl-activated PEG in a nucleophilic substitution reaction to result in a PEGylated peptide comprising the thioether linkage shown below:

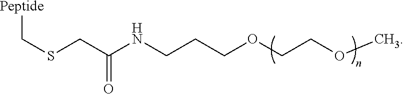

Suitable hydrophilic moieties include polyethylene glycol (PEG), polypropylene glycol, polyoxyethylated polyols (e.g., POG), polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C1-C10) alkoxy- or aryloxy-polyethylene glycol, carboxymethylcellulose, polyacetals, polyvinyl alcohol (PVA), polyvinyl pyrrolidone, poly-1,3-dioxolane, poly-1,3,6-trioxane, ethylene/maleic anhydride copolymer, poly (.beta.-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, colonic acids or other polysaccharide polymers, Ficoll or dextran and mixtures thereof. Dextrans are polysaccharide polymers of glucose subunits, predominantly linked by al-6 linkages. Dextran is available in many molecular weight ranges, e.g., about 1 kD to about 100 kD, or from about 5, 10, 15 or 20 kD to about 20, 30, 40, 50, 60, 70, 80 or 90 kD.

The hydrophilic moiety, e.g., polyethylene glycol chain, in accordance with some embodiments has a molecular weight selected from the range of about 500 to about 40,000 Daltons. In some embodiments the polyethylene glycol chain has a molecular weight selected from the range of about 500 to about 5,000 Daltons, or about 1,000 to about 5,000 Daltons. In another embodiment the hydrophilic moiety, e.g., polyethylene glycol chain, has a molecular weight of about 10,000 to about 20,000 Daltons. In yet other exemplary embodiments the hydrophilic moiety, e.g. polyethylene glycol chain, has a molecular weight of about 20,000 to about 40,000 Daltons.

Linear or branched hydrophilic polymers are contemplated. Resulting preparations of conjugates may be essentially monodisperse or polydisperse, and may have about 0.5, 0.7, 1, 1.2, 1.5 or 2 polymer moieties per peptide.

In some embodiments, the native amino acid of the peptide is substituted with an amino acid having a side chain suitable for crosslinking with hydrophilic moieties, to facilitate linkage of the hydrophilic moiety to the peptide. Exemplary amino acids include Cys, Lys, Orn, homo-Cys, or acetyl phenylalanine (Ac-Phe). In other embodiments, an amino acid modified to comprise a hydrophilic group is added to the peptide at the C-terminus.

In some embodiments, the peptide of the conjugate is conjugated to a hydrophilic moiety, e.g. PEG, via covalent linkage between a side chain of an amino acid of the peptide and the hydrophilic moiety. In some embodiments, the peptide is conjugated to a hydrophilic moiety via the side chain of an amino acid at position 16, 17, 21, 24, 29, 40, a position within a C-terminal extension, or the C-terminal amino acid, or a combination of these positions. In some aspects, the amino acid covalently linked to a hydrophilic moiety (e.g., the amino acid comprising a hydrophilic moiety) is a Cys, Lys, Orn, homo-Cys, or Ac-Phe, and the side chain of the amino acid is covalently bonded to a hydrophilic moiety (e.g., PEG).

### Multimers

The GLP-1 agonist peptides, Q may be part of a dimer, trimer or higher order multimer comprising at least two, three, or more peptides bound via a linker, wherein at least one or both peptides is a glucagon related peptide. The dimer may be a homodimer or heterodimer. In some embodiments, the linker is selected from the group consisting of a bifunctional thiol crosslinker and a bi-functional amine crosslinker. In some aspects of the invention, the monomers are connected via terminal amino acids (e.g., N-terminal or C-terminal), via internal amino acids, or via a terminal amino acid of at least one monomer and an internal amino acid of at least one other monomer. In specific aspects, the monomers are not connected via an N-terminal amino acid. In some aspects, the monomers of the multimer are attached together in a “tail-to-tail” orientation in which the C-terminal amino acids of each monomer are attached together. A conjugate moiety may be covalently linked to any of the glucagon related peptides described herein, including a dimer, trimer or higher order multimer.

### Conjugates

In some embodiments, the peptides (Q) described herein are glycosylated, amidated, carboxylated, phosphorylated, esterified, N-acylated, cyclized via, e.g., a disulfide bridge, or converted into a salt (e.g., an acid addition salt, a basic addition salt), and/or optionally dimerized, multimerized, or polymerized, or conjugated.

The present disclosure also encompasses conjugates in which Q of Q-L-Y is further linked to a heterologous moiety. The conjugation between Q and the heterologous moiety can be through covalent bonding, non-covalent bonding (e.g. electrostatic interactions, hydrogen bonds, van der Waals interactions, salt bridges, hydrophobic interactions, and the like), or both types of bonding. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other. In some aspects, the covalent bonds are peptide bonds. The conjugation of Q to the heterologous moiety may be indirect or direct conjugation, the former of which may involve a linker or spacer. Suitable linkers and spacers are known in the art and include, but not limited to, any of the linkers or spacers described herein under the sections “Acylation and alkylation”.

As used herein, the term “heterologous moiety” is synonymous with the term “conjugate moiety” and refers to any molecule (chemical or biochemical, naturally-occurring or non-coded) which is different from Q to which it is attached. Exemplary conjugate moieties that can be linked to Q include but are not limited to a heterologous peptide or polypeptide (including for example, a plasma protein), a targeting agent, an immunoglobulin or portion thereof (e.g., variable region, CDR, or Fc region), a diagnostic label such as a radioisotope, fluorophore or enzymatic label, a polymer including water soluble polymers, or other therapeutic or diagnostic agents. In some embodiments a conjugate is provided comprising Q and a plasma protein, wherein the plasma protein is selected from the group consisting of albumin, transferin, fibrinogen and globulins. In some embodiments the plasma protein moiety of the conjugate is albumin or transferin. The conjugate in some embodiments comprises Q and one or more of a polypeptide, a nucleic acid molecule, an antibody or fragment thereof, a polymer, a quantum dot, a small molecule, a toxin, a diagnostic agent, a carbohydrate, an amino acid.

### Prodrug Derivative of the GLP-1/T3 Conjugates

In accordance with one embodiment a non-enzymatic self cleaving dipeptide moiety is provided that can be covalently linked to either the GLP-1 agonist peptide or the thyroid hormone receptor ligand of the GLP-1/T3 conjugate, or both, wherein the dipeptide (and any compound linked to the dipeptide) is released from the conjugate at a predetermined length of time after exposure to physiological conditions. Advantageously, the rate of cleavage depends on the structure and stereochemistry of the dipeptide element and also on the strength of the nucleophile present on the dipeptide that induces cleavage and diketopiperazine or diketomorpholine formation. In one embodiment a complex comprising the GLP-1/T3 conjugate and a dipeptide of the structure A-B is provided, wherein A is an amino acid or a hydroxyl acid and B is an N-alkylated amino acid that is linked to the GLP-1/T3 conjugate through formation of an amide bond between B and an amine of the GLP-1/T3 conjugate. The amino acids of the dipeptide are selected such that a non-enzymatic chemical cleavage of A-B from the drug produces a diketopiperazine or diketomorpholine and the reconstituted native drug.

In one embodiment a GLP-1/T3 conjugate is provided comprising a complex having the general structure of A-B-(Q-L-Y) wherein

A is an amino acid or a hydroxyl acid;

B is an N-alkylated amino acid, wherein the dipeptide A-B is covalently linked to an amine (forming an amide bond) of either the GLP-1 agonist peptide or the thyroid hormone receptor ligand of the GLP-1/T3 conjugate. In one embodiment the side chain of A or B of the dipeptide is acylated or alkylated with a hydrocarbon chain of sufficient length to bind plasma proteins. In one embodiment the dipeptide further comprises a depot polymer linked to the side chain of A or B. Chemical cleavage of A-B from Q produces a diketopiperazine or diketomorpholine and releases the active drug to the patient in a controlled manner over a predetermined duration of time after administration.

In one embodiment the dipeptide element linked to the GLP-1/T3 conjugate comprises a compound having the general structure of Formula I:

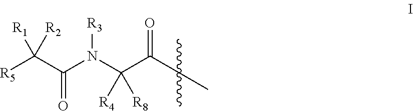

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and

R₇ is selected from the group consisting of H and OH.

In another embodiment the dipeptide element linked to the GLP-1/T3 conjugate comprises a compound having the general structure of Formula I:

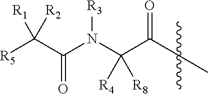

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂⁺)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R⁷, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or R₁ and R₂ together with the atoms to which they are attached form a C₃-C₁₂ cycloalkyl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-Cᵢₒ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₁ together with the atoms to which they are attached form a 4, 5 or 6 member heterocyclic ring; and

R₇ is selected from the group consisting of hydrogen, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

In one embodiment a complex is provided comprising the general structure A-B-(Q-L-Y), wherein Q-L-Y comprises any of the structures as described elsewhere in this disclosure and A-B is a dipeptide that is linked via an amide bond to an amine of the Q-L-Y conjugate. In one embodiment A-B is linked to amine present on L. In one embodiment A-B is linked to amine present on Q. In one embodiment A-B is linked to amine present on Y.

In one embodiment, a complex of the structure A-B-(Q-L-Y) is provided, wherein Q-L-Y comprises any of the structures as described elsewhere in this disclosure and wherein

A is an amino acid or a hydroxy acid;

B is an N-alkylated amino acid linked to Q or Y through an amide bond between a carboxyl moiety of B and an amine of Q or Y; and

A-B comprises the structure:

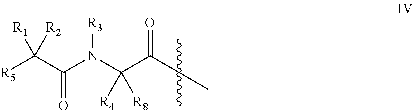

wherein
(a) R¹, R², R⁴ and R⁸ are independently selected from the group consisting of H, C1-C18 alkyl, C2-C18 alkenyl, (C1-C18 alkyl)OH, (C1-C18 alkyl)SH, (C2-C3 alkyl)SCH₃, (C1-C4 alkyl)CONH₂, (C1-C4 alkyl)COOH, (C1-C4 alkyl)NH₂, (C1-C4 alkyl)NHC(NH₂⁺)NH₂, (C0-C4 alkyl)(C3-C6 cycloalkyl), (C0-C4 alkyl)(C2-C5 heterocyclic), (C0-C4 alkyl)(C6-C10 aryl)R⁷, (C1-C4 alkyl)(C3-C9 heteroaryl), and C1-C12 alkyl(W1)C1-C12 alkyl, wherein W1 is a heteroatom selected from the group consisting of N, S and O, or
(ii) R¹ and R² together with the atoms to which they are attached form a C3-C12 cycloalkyl or aryl; or
(iii) R⁴ and R⁸ together with the atoms to which they are attached form a C3-C6 cycloalkyl;
(b) R³ is selected from the group consisting of C1-C18 alkyl, (C1-C18 alkyl)OH, (C1-C18 alkyl)NH₂, (C1-C18 alkyl)SH, (C0-C4 alkyl)(C3-C6)cycloalkyl, (C0-C4 alkyl)(C2-C5 heterocyclic), (C0-C4 alkyl)(C6-C10 aryl)R⁷, and (C1-C4 alkyl)(C3-C9 heteroaryl) or R⁴ and R³ together with the atoms to which they are attached form a 4, 5 or 6 member heterocyclic ring;
(c) R⁵ is NHR⁶ or OH;
(d) R⁶ is H, C₁-C₈ alkyl; and
(e) R⁷ is selected from the group consisting of H and OH
wherein the chemical cleavage half-life (t_{1/2}) of A-B from Q or Y is at least about 1 hour to about 1 week in PBS under physiological conditions.

In a further embodiment, A-B comprises the structure:

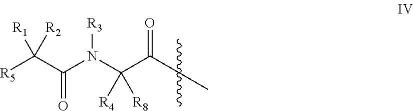

wherein

R₁ and R₈ are independently H or C₁-C₈ alkyl;

R₂ and R₄ are independently selected from the group consisting of H, C₁-C₈ alkyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, and (C₁-C₄ alkyl)(C₆ aryl)R₇;

R₃ is C₁-C₆ alkyl;

R₅ is NH₂; and

R₇ is selected from the group consisting of hydrogen, and OH.
In a further embodiment, A-B comprises the structure:

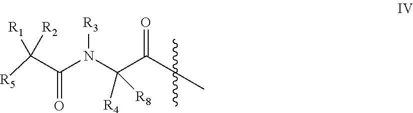

wherein

R₁ is H;

R₂ is H, C₁-C₄ alkyl, (CH₂ alkyl)OH, (C₁-C₄ alkyl)NH₂, or (CH₂)(C₆ aryl)R₇;

R₃ is C₁-C₆ alkyl;

R₄ is H, C₁-C₄ alkyl, or (CH₂)(C₆ aryl)R₇;

R₅ is NH₂

R₈ is hydrogen; and

R₇ is H or OH.

In a further embodiment, A-B comprises the structure:

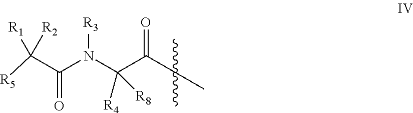

wherein

R₁ is H;

R₂ is (C₁-C₄ alkyl)NH₂;

R₃ is C₁-C₆ alkyl;

R₄ is H, C₁-C₄ alkyl, or (CH₂)(C₆ aryl)R₇;

R₅ is NH₂; and

R₈ is hydrogen.

### Exemplary Embodiments

In accordance with embodiment 1 a conjugate is provided comprising the structure Q-L-Y;

wherein

Q is a GLP-1 agonist peptide;

Y is a thyroid receptor ligand; and

L is a linking group or a bond joining Q to Y.

In accordance with embodiment 2 a conjugate of embodiment 1, wherein Y is a compound having the general structure

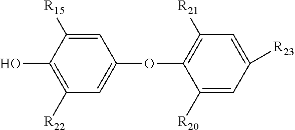

wherein

R₁₅ is C₁-C₄ alkyl, —CH₂(pyridazinone), —CH₂(OH)(phenyl)F, —CH(OH)CH₃, halo or H;

R₂₀ is halo, CH₃ or H;

R₂₁ is halo, CH₃ or H;

R₂₂ is H, OH, halo, —CH₂(OH)(C₆ aryl)F, or C₁-C₄ alkyl; and

R₂₃ is —CH₂CH(NH₂)COOH, —OCH₂COOH, —NHC(O)COOH, —CH₂COOH, —NHC(O)CH₂COOH, —CH₂CH₂COOH, and —OCH₂PO₃²⁻.

In accordance with embodiment 3 a conjugate of embodiment 1 or 2 is provided wherein

wherein

R₁₅ is C₁-C₄ alkyl, —CH(OH)CH₃, I or H

R₂₀ is I, Br, CH₃ or H;

R₂₁ is I, Br, CH₃ or H;

R₂₂ is H, OH, I, and C₁-C₄ alkyl; and

R₂₃ is —CH₂CH(NH₂)COOH, —OCH₂COOH, —NHC(O)COOH, —CH₂COOH, —NHC(O)CH₂COOH, —CH₂CH₂COOH, or —OCH₂PO₃²⁻.

In accordance with embodiment 4 a conjugate of any one of embodiments 1 to 3 is provided wherein R₁₅ is C₁-C₄ alkyl, I or H;

R₂₀ is I, Br, CH₃ or H;

R₂₁ is I, Br, CH₃ or H;

R₂₂ is H, OH, I, and C₁-C₄ alkyl; and

R₂₃ is —CH₂CH(NH₂)COOH, —OCH₂COOH, —NHC(O)COOH, —CH₂COOH, —NHC(O)CH₂COOH, —CH₂CH₂COOH, or —OCH₂PO₃²⁻.

In accordance with embodiment 5 a conjugate of any one of embodiments 1 to 4 is provided wherein Y is a compound of the general structure of Formula I:

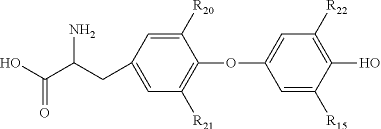

wherein

R₂₀, R₂₁, and R₂₂ are independently selected from the group consisting of H, OH, halo and C₁-C₄ alkyl; and

R₁₅ is halo or H.

In accordance with embodiment 6 a conjugate of any one of embodiments 1 to 5 is provided wherein Y is selected from the group consisting of 3,5,3′,5′-tetra-iodothyronine and 3,5,3′-triiodo L-thyronine.

In accordance with embodiment 7 a conjugate of any one of embodiments 1 to 6 is provided wherein Y is 3,5,3′-triiodo L-thyronine.

In accordance with embodiment 8 a conjugate of any one of embodiments 1 to 7 is provided wherein Y is is a compound having the general structure

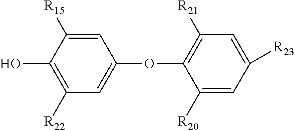

wherein

R₁₅ is isopropyl;

R₂₀ is CH₃;

R₂₁ is CH₃;

R₂₂ is H; and

R₂₃ is —OCH₂PO₃²⁻.

In accordance with embodiment 9 a conjugate of any one of embodiments 1 to 8 is provided wherein Q is a GLP-1 agonist peptide comprising the sequence

(SEQ ID NO: 43)
X₁X₂X₃GTFTSDVSX₁₂YLX₁₅X₁₆QAAX₂₀X₂₁FIX₂₄WLX₂₇X₂₈X₂₉

wherein

X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);

X₂ is selected from the group consisting of Ser, D-Ser, Ala, D-Ala, Gly, N-methyl-Ser, Aib, Val, or α-amino-N-butyric acid;

X₃ is glutamic acid, ornithine, norleucine;

X₁₂ is Lys or Arg;

X₁₅ is Asp, Glu, cysteic acid, homoglutamic acid or homocysteic acid;

X₁₆ is Aib, glutamic acid, glutamine, homoglutamic acid, homocysteic acid, threonine or glycine;

X₂₀ is Glu, Lys or Aib;

X₂₁ is Glu or Aib;

X₂₄ is Ala, Glu, Lys or Aib;

X₂₇ is Met, Val, Leu or Nle;

X₂₈ is Glu, Lys or Aib; and

X₂₉ is Gly, Gln, Asp or Glu.

In accordance with embodiment 10 a conjugate of any one of embodiments 1 to 9 is provided wherein

i) an intramolecular bridge is formed between the side chains of amino acids at positions i and i+4 of said GLP-1 agonist, wherein i is 12, 16, 20 or 24; or

ii) an Aib is present at 1, 2, 3 or 4 of positions 16, 20, 21 and 24 of said GLP-1 agonist.

In accordance with embodiment 11 a conjugate of any one of embodiments 1 to 10 is provided wherein Q is a GLP-1 agonist peptide comprising the sequence

(SEQ ID NO: 37)
X₁X₂EGTFTSDVSSYLX₁₅X₁₆QAAX₂₀X₂₁FIX₂₄WLX₂₇KG

wherein

X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);

X₂ is selected from the group consisting of Ser, D-Ser, Ala, D-Ala, Gly, N-methyl-Ser, Aib, Val, or α-amino-N-butyric acid;

X₁₅ is Asp, Glu, cysteic acid, homoglutamic acid or homocysteic acid;

X₁₆ is Gly, Glu, Lys or Aib,

X₂₀ is Glu, Ala, Lys or Aib;

X₂₁ is Glu or Aib;

X₂₄ is Ala, Glu, Lys or Aib;

X₂₇ is Met, Val, Leu or Nle.

In accordance with embodiment 12 a conjugate of any one of embodiments 9 to 11 is provided wherein

X₁₅ is Glu;

X₁₆ is Gly or Aib;

X₂₀ is Lys or Aib;

X₂₁ is Glu;

X₂₄ is Ala or Aib.

In accordance with embodiment 13 a conjugate of any one of embodiments 9 to 12 is provided wherein

X₁ is His;

X₂₀ is Lys; and

X₂₄ is Ala.

In accordance with embodiment 14 a conjugate of any one of embodiments 1 to 13 is provided wherein

Y is selected from the group consisting of 3,5,3′,5′-tetra-iodothyronine and 3,5,3′-triiodo L-thyronine; and

Q is a GLP-1 agonist peptide comprising the sequence

(SEQ ID NO: 45)
HX₂EGTFTSDVSSYLEX₁₆QAAKEFIAWLVKG
(SEQ ID NO: 38)
HAEGTFTSDVSSYLEGQAAKEFIAWLVKG
or
(SEQ ID NO: 39)
HAEGTFTSDVSSYLE(Aib)QAAKEFIAWLVKG,

wherein

X₂ is D-serine or Aib; and

X₁₆ is Gly, Glu or Aib.

In accordance with embodiment 15 a conjugate of any one of embodiments 9 to 14 is provided wherein the GLP-1 agonist peptide further comprises a C-terminal extension of X₄₀, (GPSSGAPPPSX₄₀) (SEQ ID NO: 26), SEQ ID NO: 27 (KRNRNNIAX₄₀) or SEQ ID NO: 28 (KRNRX₄₀) is bound to amino acid 29 of the GLP-1 agonist peptide through a peptide bond, wherein X₄₀ is an amino acid selected from the group consisting of Cys or Lys, optionally wherein X₄₀ is Lys, optionally wherein the GLP-1 agonist peptide comprises a C-terminal extension of SEQ ID NO: 42 (GPSSGAPPPSK).

In accordance with embodiment 18 a conjugate of any one of embodiments 1 to 15 is provided wherein the GLP-1 agonist peptide is modified to comprise a Lys at a position selected from position 24 or 29 or at position 30-41 of a C-terminal extension relative to the sequence of SEQ ID NO: 38; and the thyroid hormone receptor ligand is covalently attached to the side chain amine of a Lys at position 24 or 29 or at position 30-41 of said C-terminal extension.

In accordance with embodiment 19 a conjugate of any one of embodiments 1 to 18 is provided wherein the thyroid hormone receptor ligand is covalently attached to the side chain amine of a Lys at position 30 or 40 of said C-terminal extension.

In accordance with embodiment 20 a conjugate of any one of embodiments 1 to 19 is provided wherein the GLP-1 agonist peptide comprises the sequence

HX₂EGTFTSDVSSYLEX₁₆QAAKEFIAWLVKGGPSSGAPPPSK (SEQ ID NO: 48);

H(aib)EGTFTSDVSSYLEEQAAKEFIAWLVKGGPSSGAPPPSK-amide (SEQ ID NO: 49);

HAEGTFTSDVSSYLEGQAAKEFIAWLVKGGPSSGAPPPSK (SEQ ID NO: 46) or

HAEGTFTSDVSSYLE(Aib)QAAKEFIAWLVKGGPSSGAPPPSK (SEQ ID NO: 47), wherein

X₂ is D-serine or Aib; and

X₁₆ is Gly, Glu or Aib.

In accordance with embodiment 21 a conjugate of any one of embodiments 1 to 20 is provided wherein the thyroid hormone receptor ligand is covalently attached to the GLP-1 agonist peptide via an amino acid or dipeptide linker.

In accordance with embodiment 22 a conjugate of any one of embodiments 1 to 21 is provided wherein the thyroid hormone receptor ligand is 3,5,3′,5′-tetra-iodothyronine or 3,5,3′-triiodo L-thyronine, wherein the thyroid hormone receptor ligand is covalently linked to the side chain amine of a Lys of the GLP-1 agonist peptide through a gamma glutamic acid (γGlu) spacer added to the carboxylate of the thyroid hormone receptor.

In accordance with embodiment 23 a conjugate of any one of embodiments 1 to 22 is provided wherein L is stable in vivo, or L is hydrolyzable in vivo, or L is metastable in vivo.

In accordance with embodiment 24 a conjugate of any one of embodiments 1-22 is provided, wherein L-Y is covalently conjugated to an amino acid side chain of an amino acid at position 10, 30, 37, 38, 39, 40, 41, 42, or 43 of Q, and L is an amino acid or dipeptide.

In accordance with embodiment 22 a conjugate of any one of embodiments 1 to 24 is provided wherein L-Y comprises the structure:

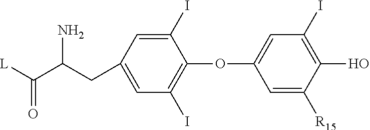

wherein

L is a bond, an amino acid, or dipeptide joining Q to Y; and

R₁₅ is H or I.

In accordance with embodiment 29 a conjugate of any one of embodiments 1 to 28 is provided wherein L is γ-Glu or the dipeptide, γ-Glu- γ-Glu.

In accordance with embodiment 30 a conjugate of any one of embodiments 1 to 29 is provided wherein L-Y comprises the structure

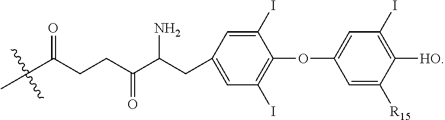

In accordance with embodiment 31 a conjugate of any one of embodiments 1 to 30 is provided wherein the GLP-1 agonist peptide comprises the sequence

(SEQ ID NO: 37)
X₁X₂EGTFTSDVSSYLX₁₅X₁₆QAAX₂₀X₂₁FIX₂₄WLX₂₇KGGPSSGAPPPSK;
or
(SEQ ID NO: 49)
H(aib)EGTFTSDVSSYLEEQAAKEFIAWLVKGGPSSGAPPPSK-amide

wherein

X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);

X₂ is selected from the group consisting of Ser, D-Ser, Ala, D-Ala, Gly, N-methyl-Ser, Aib, Val, or α-amino-N-butyric acid;

X₁₅ is Asp, Glu, cysteic acid, homoglutamic acid or homocysteic acid;

X₁₆ is Gly, Glu, Lys or Aib,

X₂₀ is Glu, Ala, Lys or Aib;

X₂₁ is Glu or Aib;

X₂₄ is Ala, Glu, Lys or Aib; and

X₂₇ is Met, Val, Leu or Nle.

In accordance with embodiment 32, a conjugate of any one of embodiments 1 to 31 is provided wherein L-Y is conjugated to an amino acid side chain of the GLP-1 agonist peptide at position 40.

In accordance with embodiment 33 a conjugate of any one of embodiments 9 to 31 is provided wherein

X₁ is His;

X₂ is selected from the group consisting of Ser, D-Ser, Ala, D-Ala, Gly, N-methyl-Ser, Aib, Val, or α-amino-N-butyric acid;

X₁₅ is Glu; and

X₁₆ is Gly or Aib,

X₂₀ is Lys or Aib;

X₂₁ is Glu or Aib;

X₂₄ is Ala or Aib; and

X₂₇ is Met, Val, Leu or Nle.

In accordance with embodiment 34 a conjugate of any one of embodiments 1 to 33 is provided wherein the GLP-1 agonist peptide comprises the sequence

HX₂EGTFTSDVSSYLE(Aib)QAAKEFIAWLVKG (SEQ ID NO: 50)

wherein

X₂ is Ser, D-Ser, or Aib.

In accordance with embodiment 35 a conjugate of any one of embodiments 1 to 34 is provided wherein

A is an amino acid or a hydroxy acid;

B is an N-alkylated amino acid linked to Q or Y through an amide bond between a carboxyl moiety of B and an amine of Q or Y; and

A-B comprises the structure:

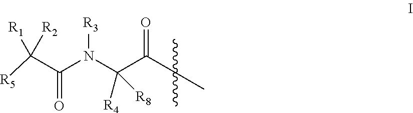

wherein

(a) R¹, R², R⁴ and R⁸ are independently selected from the group consisting of H, C1-C18 alkyl, C2-C18 alkenyl, (C1-C18 alkyl)OH, (C1-C18 alkyl)SH, (C2-C3 alkyl)SCH₃, (C1-C4 alkyl)CONH₂, (C1-C4 alkyl)COOH, (C1-C4 alkyl)NH₂, (C1-C4 alkyl)NHC(NH₂⁺)NH₂, (C0-C4 alkyl)(C3-C6 cycloalkyl), (C0-C4 alkyl)(C2-C5 heterocyclic), (C0-C4 alkyl)(C6-C10 aryl)R⁷, (C1-C4 alkyl)(C3-C9 heteroaryl), and C1-C12 alkyl(W1)C1-C12 alkyl, wherein W1 is a heteroatom selected from the group consisting of N, S and O, or

(ii) R¹ and R² together with the atoms to which they are attached form a C3-C12 cycloalkyl or aryl; or

(iii) R⁴ and R⁸ together with the atoms to which they are attached form a C3-C6 cycloalkyl;

(b) R³ is selected from the group consisting of C1-C18 alkyl, (C1-C18 alkyl)OH, (C1-C18 alkyl)NH₂, (C1-C18 alkyl)SH, (C0-C4 alkyl)(C3-C6)cycloalkyl, (C0-C4 alkyl)(C2-C5 heterocyclic), (C0-C4 alkyl)(C6-C10 aryl)R⁷, and (C1-C4 alkyl)(C3-C9 heteroaryl) or R⁴ and R³ together with the atoms to which they are attached form a 4, 5 or 6 member heterocyclic ring;

(c) R⁵ is NHR⁶ or OH;

(d) R⁶ is H, C₁-C₈ alkyl; and

(e) R⁷ is selected from the group consisting of H and OH

wherein the chemical cleavage half-life (t_{1/2}) of A-B from Q or Y is at least about 1 hour to about 1 week in PBS under physiological conditions.

In accordance with embodiment 36 a conjugate of embodiment 35 is provided, wherein
R₁ and R₈ are independently H or C₁-C₈ alkyl;
R₂ and R₄ are independently selected from the group consisting of H,
C₁-C₈ alkyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, and (C₁-C₄ alkyl)(C₆ aryl)R₇;
R₃ is C₁-C₆ alkyl;
R₅ is NH₂; and
R₇ is selected from the group consisting of hydrogen, and OH.

In accordance with embodiment 37 a conjugate of any one of embodiments 35 to 36 is provided wherein
R₁ is H;
R₂ are independently H, C₁-C₄ alkyl, (CH₂ alkyl)OH, (C₁-C₄ alkyl)NH₂, or (CH₂)(C₆ aryl)R₇;
R₃ is C₁-C₆ alkyl;
R₄ is H, C₁-C₄ alkyl, or (CH₂)(C₆ aryl)R₇;

R₈ is hydrogen; and
R₅ is an amine.

In accordance with embodiment 38 a conjugate of any one of embodiments 1 to 37 is provided, further comprising an amino acid side chain on Q covalently attached to an acyl group or an alkyl group via an alkyl amine, amide, ether, ester, thioether, or thioester linkage, which acyl group or alkyl group is non-native to a naturally occurring amino acid.

In accordance with embodiment 39 a conjugate of embodiment 38 is provided wherein the amino acid to which the acyl or alkyl group is attached is at a position corresponding to position 10, 20, 24, 30, 37, 38, 39, 40, 41, 32, or 43 of a sequence comprising native glucagon, or the C-terminal amino acid.

In accordance with embodiment 40 a conjugate of any one of embodiments 38 to 39 is provided wherein the amino acid to which the acyl or alkyl group is attached is at a position corresponding to position 10 of a sequence comprising native glucagon.

In accordance with embodiment 41 a conjugate of any one of embodiments 38 to 40 is provided wherein the acyl group or the alkyl group is attached to the side chain of the amino acid through a spacer and comprises carboxylate at the free end of the acyl group.

In accordance with embodiment 42 a conjugate of any one of embodiments 38 to 41 is provided wherein the spacer is an acidic amino acid or an acidic dipeptide.

In accordance with embodiment 43 pharmaceutical composition comprising a conjugate of any one of embodiments 1 to 42 is provided wherein said composition comprises a pharmaceutically acceptable carrier.

In accordance with embodiment 44 a method for treating a disease or medical condition in a patient, wherein the disease or medical condition is selected from the group consisting of hyperlipidemia, metabolic syndrome, diabetes, obesity, liver steatosis, and chronic cardiovascular disease, is provided wherein a conjugate of any one of embodiments 1 to 21 is administering to the patient in an amount effective to treat the disease or medical condition.

### Example 1

Generation of GLP-1 and Thyroid Hormone Conjugates

### Materials and Methods

### Peptide Synthesis.

Peptide backbones were synthesized by standard fluorenylmethoxycarbonyl (Fmoc)-based solid phase peptide synthesis using 0.1 mmol Rink amide 4-methylbenzhydrylamine (MBHA) resin (Midwest Biotech) on an Applied Biosystems 433A peptide synthesizer. The automated synthesizer utilized 20% piperidine in N-methyl-2-pyrrolidone (NMP) for N-terminal amine deprotection and diisopropylcarbodiimide (DIC)/6-CI-HOBt for amino acid coupling.

### Synthesis of GLP-1/T3 Conjugate (IUB686).

A 1:1 molar ratio of 3, 5, 3′-triiodothyronine and di-tert-butyl dicarbonate was dissolved in dioxane/water (4:1,v:v) in the presence of an ice bath with an addition of 0.1 equivalent of triethylamine (TEA). The reaction was stirred for 30 min at 30° C. and then at room temperature for 30 hours, during which the progress of the reaction was monitored by analytical HPLC. Upon completion, the pH of the solution was lowered to 4.0 with 0.1 M hydrochloride (HCl) acid, subsequently treated it repetitively with dichloromethane (DCM) to extract desired product. The organic phase was collected, combined and evaporated in vacuum to afford crude product Boc-T3-OH with good purity.

The peptide backbone synthesized contained a C-terminal N′-methyltrityl-L-lysine (Lys(Mtt)-OH) moiety, whose side chain was orthogonally deprotected by four sequential 10-min treatments with 1% trifluoroacetic acid (TFA), 2% triisopropylsilane (TIS) in DCM to expose an amine as a site for T3 conjugation. The peptidyl-resin was then mixed with a tenfold excess of Fmoc-L-Glu-OtBu(rE) activated by 3-(diethoxyphosphoryloxy)-1,2,3-benzotriazin-4(3H)-one/N,Ndiisopropylethylamine (DEPBT/DIEA) in dimethylformide (DMF) for 2 hours. The completion of the coupling was confirmed by Kaiser test, after which the resin was washed and treated with 20% piperidine in DMF to remove the Fmoc protecting group located at the side chain of the γGlu residue. Subsequently, the peptidyl-resin was reacted with a fivefold excess of crude Boc-T3-OH combined with DEPBT/DIEA in DMF for 2 hours to facilitate T3 conjugation to peptide backbone. Afterwards, the resin were treated with TFA cleavage cocktail containing TFA/anisole/TIS/H2O (85:5:5:5) for 2 hours at room temperature to release conjugate from solid support. Cleaved and fully deprotected conjugate was precipitated and washed with chilled diethyl-ether. The crude conjugates was dissolved in 15% aqueous acetonitrile containing 15% acetic acid and purified by preparative reversed-phase HPLC utilizing a linear gradient of buffer B over buffer A (A:10% aqueous acetonitrile, 0.1% TFA; B:100% acetonitrile, 0.1% TFA) on an axia-packed phenomenex luna C18 column (250×21.20 mm) to afford the desired conjugate with carboxyl coupling of T3 to GLP-1 analogs.

### Synthesis of GLP-1/iT3 Conjugate (IUB687).

3, 5, 3′-triiodothyronine (CHEM-IMPEX INT'L INC.) was solubilized in tert-butyl acetate in the presence of 0.1 equivalent of perchloric acid (HClO₄). The mixture was stirred at 0° C. for 2 hours and at room temperature for 14 hours. Upon completion, the mixture was washed with water and ethyl acetate, treated with 10M sodium hydroxide (NaOH) until pH of the solution reached 9. Subsequently, the mixture was extracted with DCM. The combined organic phase was dried by magnesium sulfate (MgSO₄) and evaporated in vacuum to obtain the desired product NH₂-T3-OtBu. NH₂-T3-OtBu and succinic anhydride were mixed in anhydrous DMF with 0.1 equivalent of DIEA. The reaction was stirred at room temperature for 48 hours. The OH-Suc-T3-OtBu product was obtained followed the same workup as described for NH2-T3-OtBu. Crude OH-Suc-T3-OtBu was dissolved in 15% aqueous acetonitrile containing 15% acetic acid and purified by semi-preparative reversed-phase HPLC using a linear gradient of buffer B over buffer A on an axia-packed phenomenex Luna C18 column (250×21.20 mm). Equimolar equivalents of HO-Suc-T3-OtBu, DEPBT and DIEA were solubilized in DMF and directly added to the peptidyl-resin. The reaction was gently agitated at room temperature for two hours and was monitored by Kaiser test. The peptidyl resins were treated with TFA cleavage cocktail containing TFA/anisole/tTIS/H2O (85:5:5:5) for 2 hours at room temperature to cleave conjugate from solid support. Cleaved conjugate was precipitated and washed with chilled diethyl-ether. IUB687 was dissolved and purified by reversed-phase HPLC using the condition described above.

### Human GLP-1 Receptor Activation.

Each peptide or conjugate was individually tested for its ability to activate the human GLP-1R through a cell-based luciferase reporter gene assay that indirectly measures cAMP induction. Human embryonic kidney (HEK293) cells were co-transfected with GLP-1R cDNA (zeocin-selection) and a luciferase reporter gene construct fused to a cAMP response element (CRE) (hygromycin B-selection). Cells were seeded at a density of 22,000 cells per well and serum deprived for 16 h in DMEM (HyClone) supplemented with 0.25% (vol/vol) bovine growth serum (BGS) (HyClone). Serial dilutions of the peptides were added to 96-well cell-culture treated plates (BD Biosciences) containing the serum-deprived, co-transfected HEK293 cells, and incubated for 5 h at 37° C. and 5% CO₂ in a humidified environment. To stop the incubation, an equivalent volume of Steady Lite HTS luminescence substrate reagent (Perkin Elmer) was added to the cells to induce lysis and expose the lysates to luciferin. The cells were agitated for 5 min and stored for 10 min in the dark.

Luminescence was measured on a MicroBeta-1450 liquid scintillation counter (Perkin-Elmer). Luminescence data was graphed against concentration of peptide and EC50 values were calculated using Origin software (OriginLab).

### Wild-Type Mice for Pharmacology Studies.

For pharmacology studies on energy metabolism in obese mice (DIO mice), male C57Bl/6j mice (Jackson Laboratories) were fed a diabetogenic diet (Research Diets D12331), which is a high-sucrose diet with 58% kcal from fat 25.5% kcal from carbohydrates, and 16.4% kcal from protein. Dietary challenges began at 8 weeks of age. HFHSD mice were single- or group-housed on a 12:12-h light-dark cycle at 22° C. with free access to food and water. Mice were maintained under these conditions for a minimum of 16 weeks before initiation of pharmacological studies and were between the ages of 6 months to 12 months old. All injections and tests were performed during the light cycle. Compounds were administered in a vehicle of 1% Tween-80 and 1% DMSO and were given by daily subcutaneous injections at the indicated doses at a volume of 5 μl per g body weight. Mice were randomized and evenly distributed to test groups according to body weight and body composition. If ex vivo molecular biology/histology/biochemistry analyses were performed, the entire group of mice for each treatment was analyzed and scored in a blinded fashion.

### Genetically-Modified Mouse Lines.

Global Glp-1r mice were obtained from a commercial source. CNS-specific Glplr−/− mice were generated by crossing Glplr flox/flox mice with Nestin-Cre mice. All genetically modified mice were maintained on the HFHSD for 12 weeks prior to initiation of treatment. All mice were single- or group-housed on a 12:12-h light-dark cycle with free access to food and water.

### Rodent Pharmacological and Metabolism Studies.

Compounds were administered by repeated subcutaneous injections in the middle of the light phase at the indicated doses with the indicated durations. Co-administration of compounds was administered by single formulated injections. Body weights and food intake were measured every day or every other day after the first injection. All studies with wild-type mice were performed with a group size of n=8 or greater using mice on a C57B16j background.

For assessment of intraperitoneal glucose tolerance during chronic treatment, the challenge tests were performed at least 24 hours after the last administration of compounds. The investigators were not blinded to group allocation during the in vivo experiments or to the assessment of longitudinal endpoints. All rodent studies were approved by and performed according to the guidelines of the Institutional Animal Care and Use Committee of the Helmholtz Center Munich, University of Cincinnati, Universite de Lyon, and in accordance with guidelines of the Association for the Assessment and Accreditation of Laboratory and Animal Care (AAALAC # Unit Number: 001057) and appropriate federal, state and local guidelines, respectively.

### Body Composition Measurements.

Whole-body composition (fat and lean mass) was measured using nuclear magnetic resonance technology (EchoMRI).

### Indirect Calorimetry.

Energy intake, energy expenditure, respiratory exchange ratio, and home-cage activity were assessed using a combined indirect calorimetry system (TSE Systems). O2 consumption and CO2 production were measured every 10 min for a total of up to 160 h (after 24 h of adaptation) to determine the respiratory quotient and energy expenditure after an initial treatment regimen. Food intake was determined continuously for the same time as the indirect calorimetry assessments by integration of scales into the sealed cage environment. Home-cage locomotor activity was determined using a multidimensional infrared light beam system with beams scanning the bottom and top levels of the cage, and activity being expressed as beam breaks.

### Blood Parameters.

Blood was collected at the indicated times from tail veins or after euthanasia using EDTA-coated microvette tubes (Sarstedt), immediately chilled on ice, centrifuged at 5,000 g and 4° C., and plasma was stored at −80° C. Plasma cholesterol and triglycerides were measured using enzymatic assay kits (Thermo Fisher). All assays were performed according to the manufacturers' instructions.

### Results

### GLP-1/T3 Lowers Body Weight Through Anorectic and Thermogenic Actions

GLP-1 analogs have repeatedly demonstrated a potent ability to lower body weight, and this phenomenon is primarily driven by centrally derived anorectic mechanisms. On the contrast, thyroid hormones, including the endogenous hormone T3, have repeatedly demonstrated the potent ability to lower body weight across species primarily through energy expenditure mechanisms. A portion of the thermogenic actions of thyroid hormones has been attributed to centrally derived mechanisms to increase sympathetic/catecholamertergic outflow to brown adipose tissue (BAT) through uncoupling protein-1 (UCP-1) dependent effects. A series of GLP-1 and T3 conjugates were generated that are fully potent at the human GLP-1R with negligible activity at the human glucagon receptor (GcgR; Table 1). The structures of IUB48, IUB686 and IUB687 are as follows:

### IUB 48 HaibEGTFTSDVSSYLEEQAAKEFIAWLVKGGPSSGAPPPSK-amide (SEQ ID NO: 49);

IUB686 IUB48-(gE-T3-NH2)-amide (T3 linked via gamma Glu to Lys at position 40);
IUB687 IUB48-(Suc-T3-OH)-amide (T3 linked via succinic anhydride to Lys at position 40).

TABLE 1

| | EC₅₀ (nM) | |
|---|---|---|
| Peptide | hGLP-1R | hGcgR |
| Glucagon | 1.595 | 0.08 |
| GLP-1 | 0.037 | >100 |
| IUB48 | 0.008 | >100 |
| IUB686 | 0.010 | >100 |
| IUB687 | 0.012 | >100 |

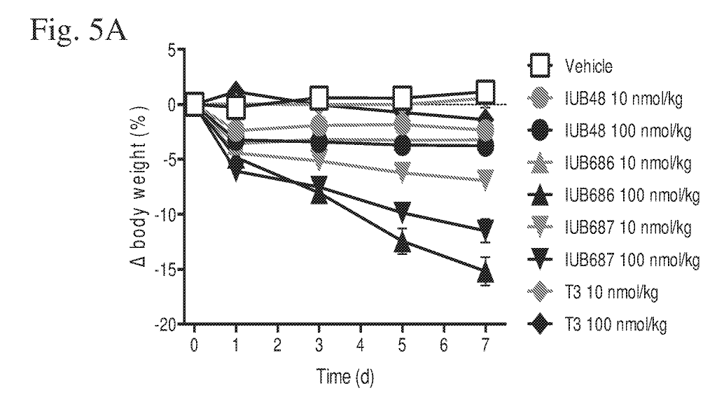

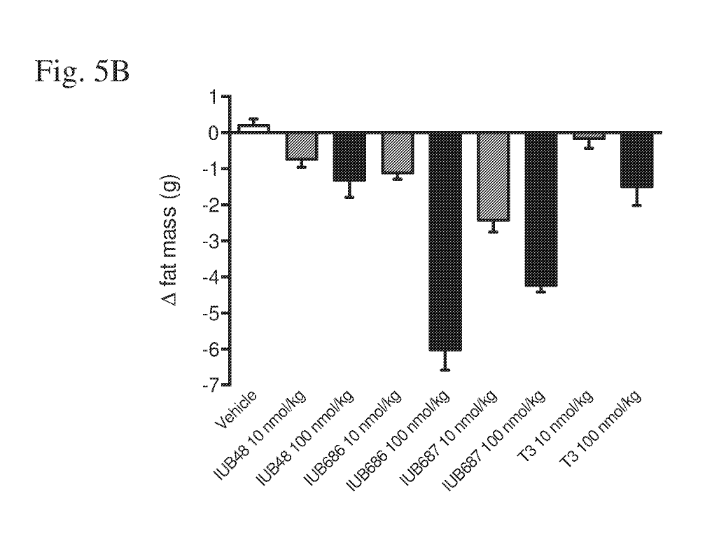

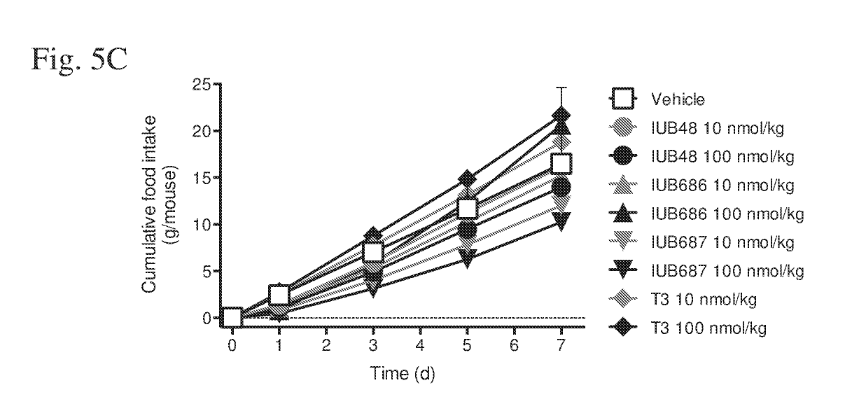

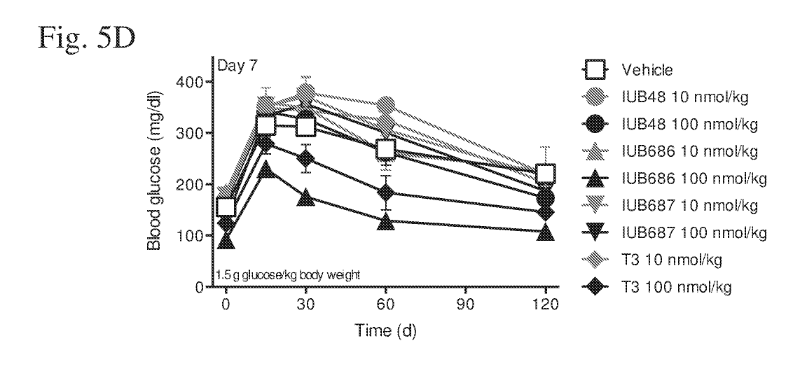

To explore the weight-lowering properties of coordinated GLP-1 and thyroid hormone signaling, we explored the weight-lowering capacity of GLP-1/T3 conjugates in diet-induced obese (DIO) mice maintained on a high-fat, high-sugar diet (HFHSD) compared to mono-agonist controls at equimolar doses. At the doses used herein, T3 had the appropriate GLP-1 analog control (IUB48), had minimal impact on body weight. However, both of the GLP-1/T3 conjugates (IUB686, GLP-1/T3; IUB687, GLP-1/iT3) significantly lowered body weight in a dose-dependent manner, demonstrating a synergistic quality of coordinated signaling governed by these two hormones. At an equimolar dose of 100 nmoles/kg, an absolute decrease in body weight of 15% and 11% from baseline was observed after a week of daily treatment with IUB686 and IUB687, respectively (FIG. 5A). The loss of body weight caused by both conjugates was due to a loss of fat mass (FIG. 5B). Food intake was increased by systemic T3 treatment, recapitulating the hyperphagia associated with central hyperthyroidism in rodents (FIG. 5C). Despite the 11% decrease in body weight following treatment with IUB687, no reductions in food intake were observed with this compound (FIG. 5C). However, treatment IUB686 resulted in a dose-dependent reduction in food intake (FIG. 5C). In respect to conjugate therapies, only treatment with the higher dose of IUB686 resulted in an improved glucose tolerance (FIG. 5D), which is significantly greater than the improved glycemic control achieved by equimolar systemic T3. Based on these preliminary findings in which IUB686 was imparting substantial food intake inhibition, we decided to more thoroughly test the potency, efficacy, safety, and mechanisms moving forward.

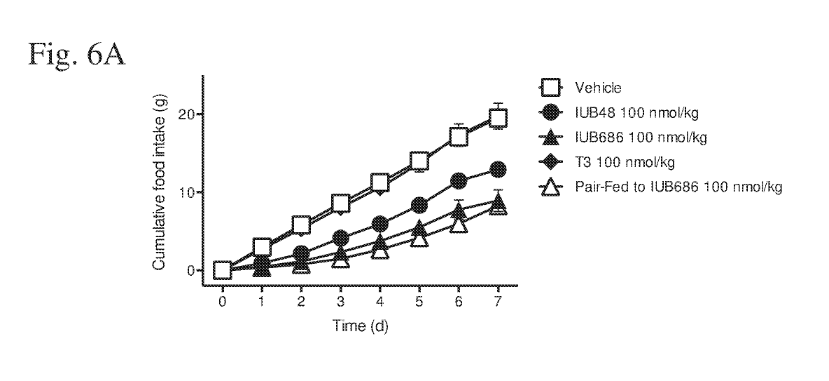

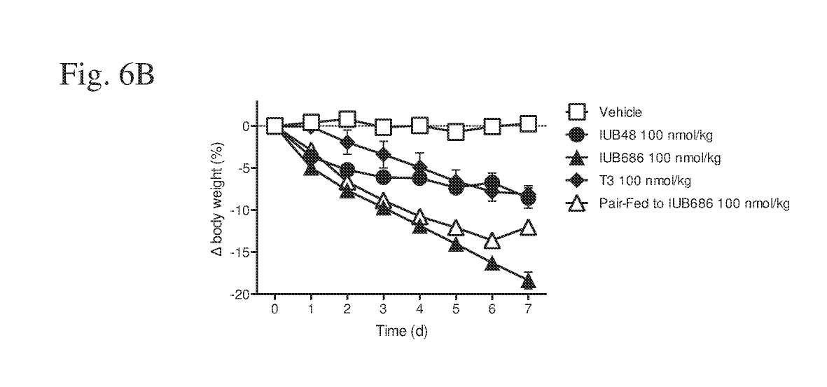

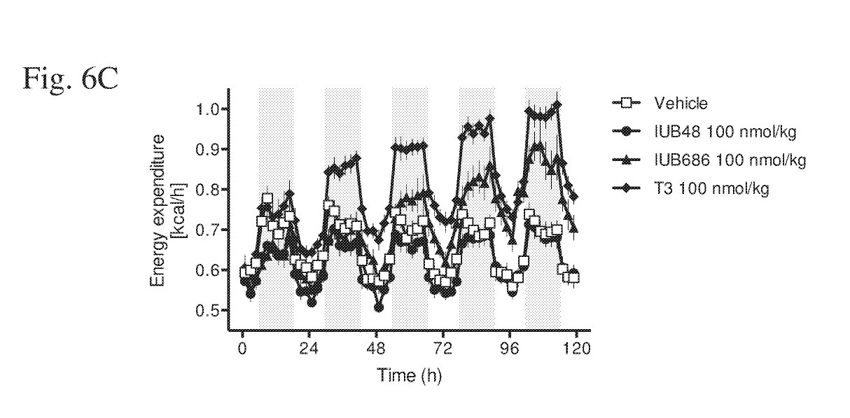

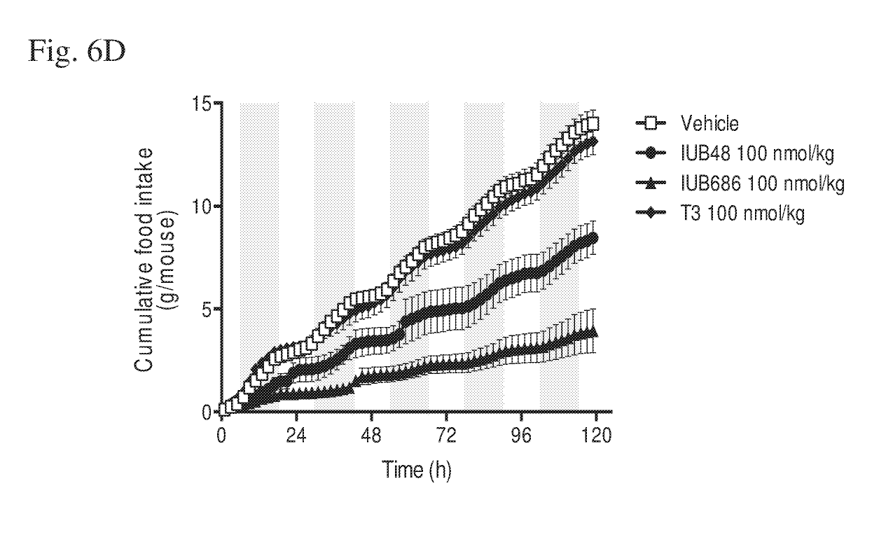

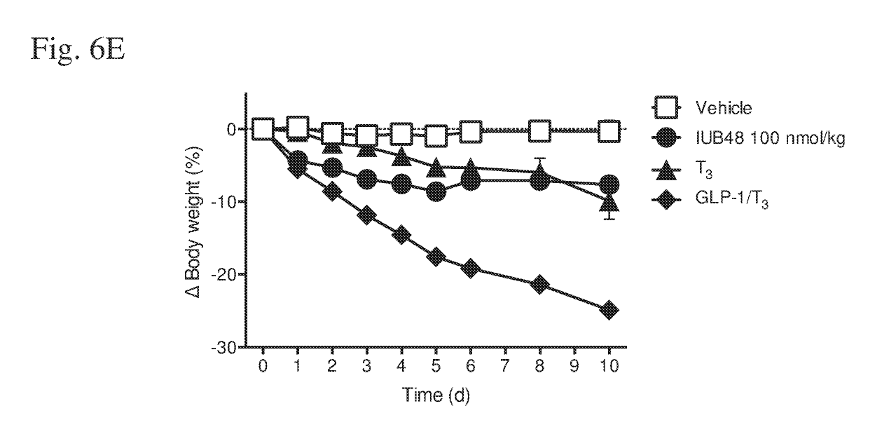

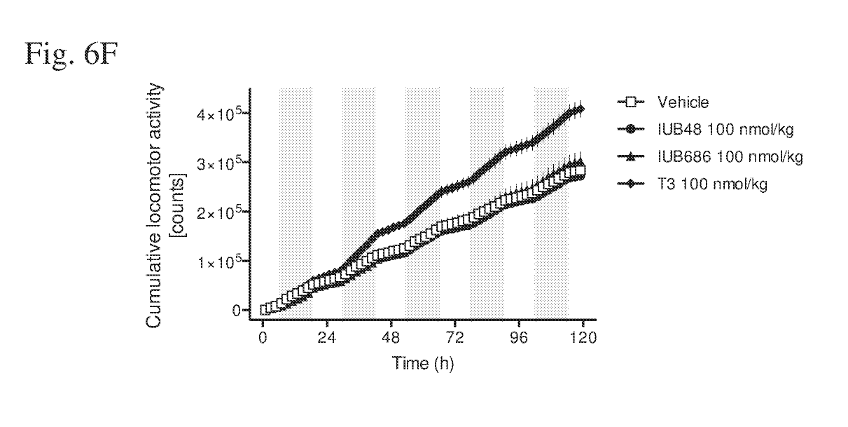

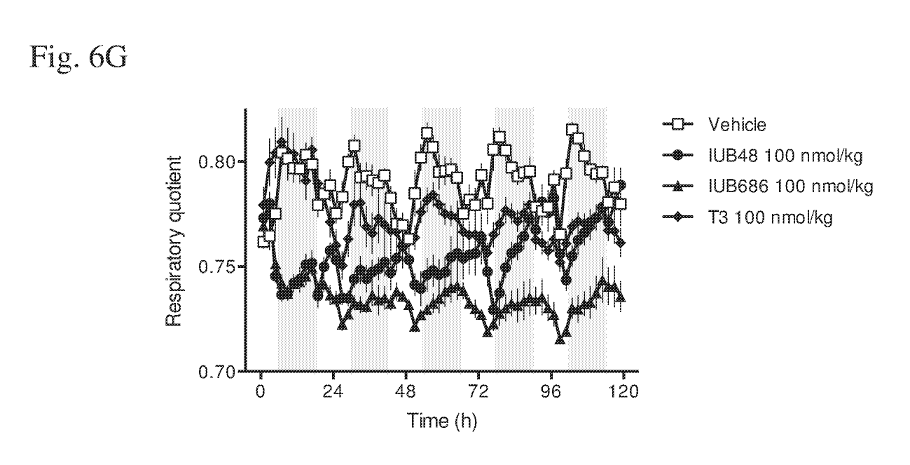

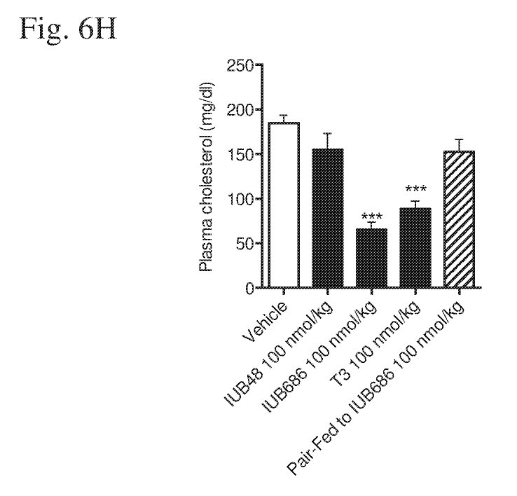

To indirectly determine if IUB686 is promoting body weight loss through mechanisms independent of food intake, we compared the metabolic benefits delivered by IUB686 to those of its caloric mice. Compared to those mice pair-fed to receive the same amount of food as those mice that received IUB686 (FIG. 6A), we see that IUB686 treatment results in greater body weight lowering than the pair-fed controls (FIG. 6B). This suggests that energy expenditure mechanisms are contributing to the body weight lowering effect observed with IUB686. We quantified the amount of energy expenditure imparted by coupling T3 to GLP-1 using indirect calorimetry. Both T3 alone and IUB686 (GLP-1/T3) substantially increased whole-body energy expenditure but to differing degrees with IUB686 causing significantly less energy expenditure than systemic T3 (FIG. 6C). This implements that the preferential targeting of T3 that is governed by GLP-1 partially engages the same thermogenic machinery as systemic T3, or that GLP-1 mediated targeting elicits thermogenic mechanisms distinct from when T3 alone is administered as is.

Interestingly, the thermogenic effect of IUB686 takes time to set in compared to systemic T3, suggesting that the unique signaling induced by coordinated GLP-1 and T3 signaling empowers or promotes reciprocal thermogenic actions. With systemic T3 alone, the observed hyperphagia (FIG. 6D) partially compensated for the increased energy expenditure, which is in magnitude is greater than achieved by IUB686 therapy. This resulted in in less body weight loss delivered by T3 compared to that achieved by IUB686 therapy, which itself drove a negative energy balance resulting in a loss of body weight (FIG. 6E). Furthermore, systemic T3 significantly increased home cage activity (FIG. 6F), which paralleled the observed increase in energy expenditure. Unlike mono-therapy with T3 however, the conjugate did not cause an increase in ambulatory activity (FIG. 6F), demonstrating that altered activity is not contributing to the enhanced energy expenditure caused by the GLP-1 based conjugate. Additionally, IUB686 caused a decrease in the respiratory exchange ratio (RER) (FIG. 6G) to a greater extent than what is achieved by GLP-1 monotherapy (IUB48), indicating that the coordinated action of the two constituents shifted nutrient partitioning to promote fat utilization.

As both body weight loss induced by caloric restriction and hyperthyroidism can improve circulating levels of cholesterol, we observe that IUB686 improved plasma cholesterol levels independent of caloric restriction as pair-fed mice did not show an improved effect (FIG. 6G). The magnitude of the cholesterol lowering effect is also greater than that achieved by systemic T3 alone (FIG. 6H). However, unlike systemic T3 therapy, which induced cardiac hypertrophy, IUB686 did not increase raw heart weight (FIG. 6I). This indicates that we have improved the therapeutic index of T3 by covalently attaching it to GLP-1, which drives the preferential delivery to metabolically-relevant tissues harboring the GLP-1 receptor.

### Discrete Aspects of the Metabolic Benefits are Co-Mediated by GLP-1R and THRs.

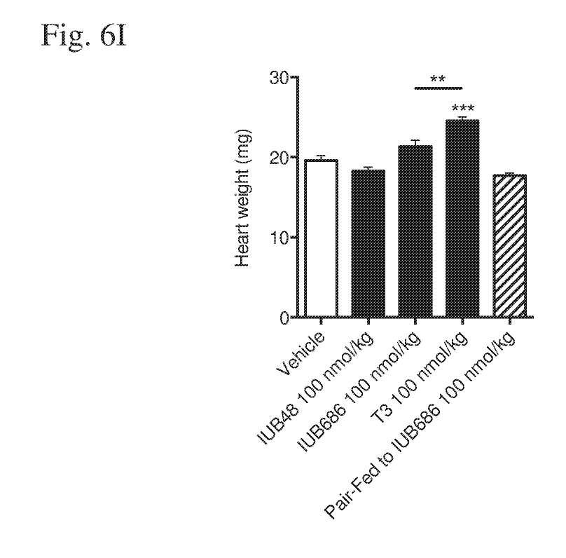

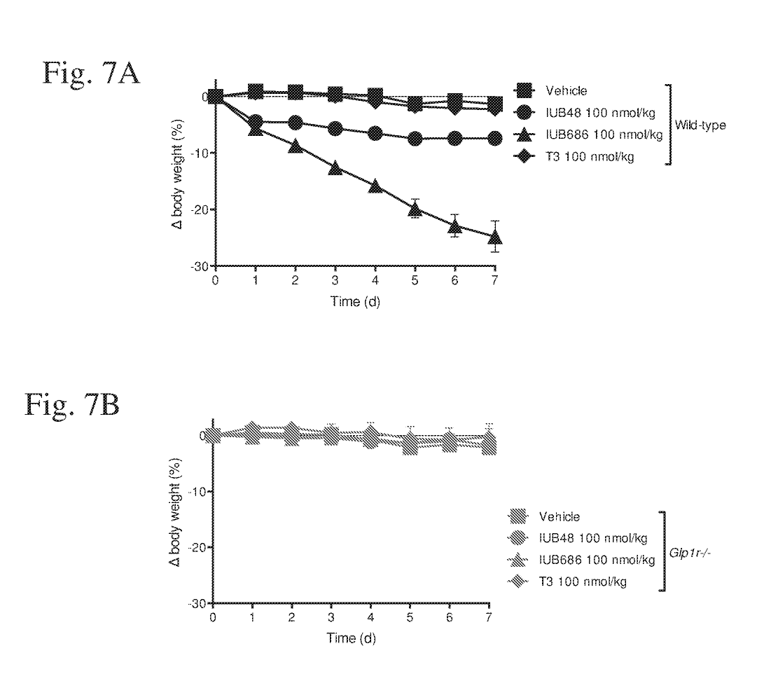

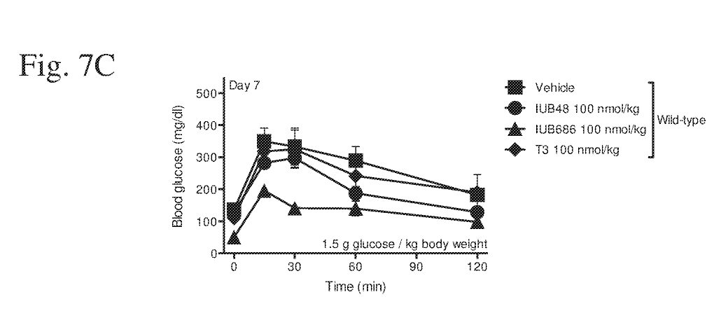

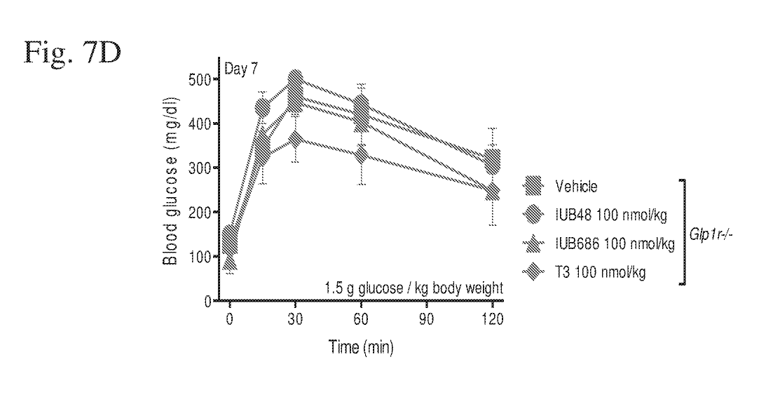

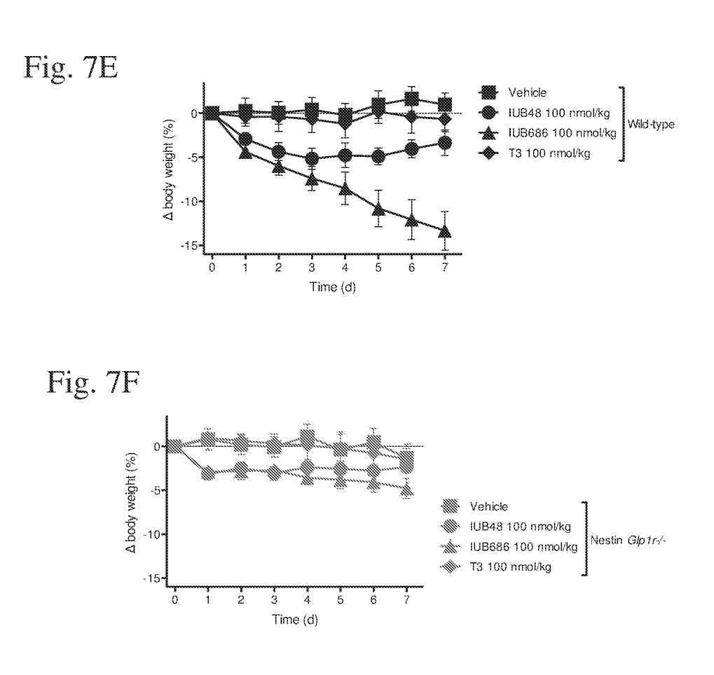

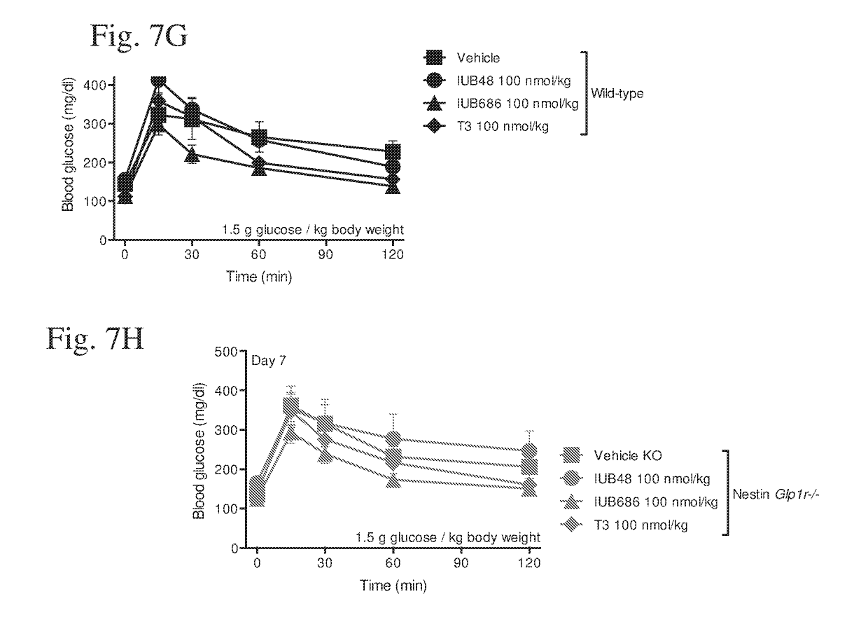

To exclude off-target effects and examine the contribution of each component within IUB686 to the metabolic benefits, particularly on body weight and glucose tolerance, we administered IUB686 to global GLP-1R−/− mice made obese by HFHSD feeding. In these global GLP-1R−/− mice, the effects of IUB686 to lower body weight (FIG. 7A) and improve glucose tolerance (FIG. 7B) were completely lost relative to wild-type mice. The complete loss of an effect in global GLP-1R−/− mice confirmed the target specificity of the conjugate, and demonstrated that GLP-1 activity is essential for sufficient T3 delivery and for coordinating the improved energy homeostasis. Furthermore, the lack of effects in GLP-1R−/− mice demonstrates that the T3 moiety is not prematurely separating from the peptide in circulation.

To ascertain the contribution of central GLP-1R signaling to the metabolic benefits of IUB686, we tested the GLP-1/T3 conjugate in mice with the genetic deletion of GLP-1R in the central nervous system (CNS) that were maintained on a HFHSD. In these CNS-specific GLP-1R−/− mice, the effects of IUB686 to lower body weight were substantially blunted (FIG. 7C). Although blunted, the weight-lowering effects of IUB686 were not completely abolished in the CNS-specific GLP-1R−/− mice, and the magnitude of the weight loss is comparable to what is achieved by the GLP-1R mono-agonist (FIG. 7C). This demonstrates centrally mediated mechanisms are responsible for the enhanced body-weight lowering achieved when covalently attaching a T3 moiety to GLP-1. However, this is not the case for the improved glycemic control observed with GLP-1/T3 conjugates. The improved glucose tolerance delivered by chronic IUB686 treatment seen in wild-type mice is preserved in CNS-specific GLP-1R−/− mice (FIG. 7D), indicating that peripheral mechanisms are facilitating the better glycemic control governed by coordinated GLP-1 and T3 signaling. These peripheral mechanisms are likely originating from actions in pancreatic β cells and brown adipose tissue, and are currently under exploration.

1. A conjugate comprising the structure Q-L-Y;
wherein
Q is a GLP-1 agonist peptide comprising
A) the sequence
(SEQ ID NO: 43)
X₁X₂X₃GTFTSDVSX₁₂YLX₁₅X₁₆QAAX₂₀X₂₁FIX₂₄WLX₂₇X₂₈X₂₉
wherein
X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);
X₂ is selected from the group consisting of Ser, D-Ser, Ala, D-Ala, Gly, N-methyl-Ser, Aib, Val, or α-amino-N-butyric acid;
X₃ is glutamic acid, ornithine, norleucine;
X₁₂ is Lys or Arg;
X₁₅ is Asp, Glu, cysteic acid, homoglutamic acid or homocysteic acid;
X₁₆ is Aib, glutamic acid, glutamine, homoglutamic acid, homocysteic acid, threonine or glycine;
X₂₀ is Glu, Lys or Aib;
X₂₁ is Glu or Aib;
X₂₄ is Ala, Glu, Lys or Aib;
X₂₇ is Met, Val, Leu or Nle;
X₂₈ is Glu, Lys or Aib; and
X₂₉ is Gly, Gln, Asp or Glu; or
B) the sequence
(SEQ ID NO: 37)
X₁X₂EGTFTSDVSSYLX₁₅X₁₆QAAX₂₀X₂₁FIX₂₄WLX₂₇KG
wherein
X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);
X₂ is selected from the group consisting of Ser, D-Ser, Ala, D-Ala, Gly, N-methyl-Ser, Aib, Val, or α-amino-N-butyric acid;
X₁₅ is Asp, Glu, cysteic acid, homoglutamic acid or homocysteic acid;
X₁₆ is Gly, Glu, Lys or Aib,
X₂₀ is Glu, Ala, Lys or Aib;
X₂₁ is Glu or Aib;
X₂₄ is Ala, Glu, Lys or Aib;
X₂₇ is Met, Val, Leu or Nle;
Y is a thyroid receptor ligand having the general structure of:
I) 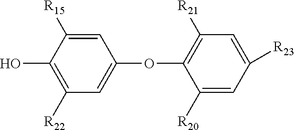
wherein
R₁₅ is C₁-C₄ alkyl, —CH₂(pyridazinone), —CH₂(OH)(phenyl)F, —CH(OH)CH₃, halo or H;
R₂₀ is halo, CH₃ or H—;
R₂₁ is halo, CH₃ or H—;
R₂₂ is H, OH, halo, —CH₂(OH)(C₆ aryl)F, or C₁-C₄ alkyl; and
R₂₃ is —CH₂CH(NH₂)COOH, —OCH₂COOH, —NHC(O)COOH, —CH₂COOH, —NHC(O)CH₂COOH, —CH₂CH₂COOH, and —OCH₂PO₃²⁻; or
II) 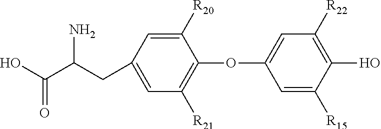
wherein
R₂₀, R₂₁ and R₂₂ are independently selected from the group consisting of H, OH, halo and C₁-C₄ alkyl; and
R₁₅ is halo or H; or
III) 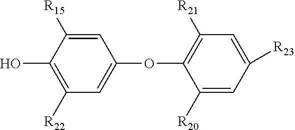
wherein
R₁₅ is isopropyl;
R₂₀ is CH₃;
R₂₁ is CH₃;
R₂₂ is H; and
R₂₃ is —OCH₂PO₃²; and
L is a linking group or a bond joining Q to Y.

2-4. (canceled)

5. The conjugate of claim 1 wherein Y is a compound of the general structure of Formula I: 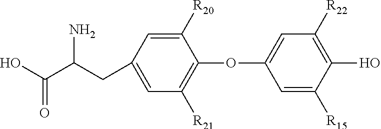
wherein R₂₀, R₂₁, and R₂₂ are independently I or Cl; and
R₁₅ is H Cl or I.

6. The conjugate of claim 1 wherein Y is selected from the group consisting of 3,5,3′,5′-tetra-iodothyronine and 3,5,3′-triiodo L-thyronine.

7-11. (canceled)

12. The conjugate of claim 1, wherein Q is a GLP-1 agonist peptide comprising the sequence
(SEQ ID NO: 37)
X₁X₂EGTFTSDVSSYLX₁₅X₁₆QAAX₂₀X₂₁FIX₂₄WLX₂₇KG
wherein
X₁ is His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);
X₂ is selected from the group consisting of Ser, D-Ser, Ala, D-Ala, Gly, N-methyl-Ser, Aib, Val, or α-amino-N-butyric acid;
X₁₅ is Glu;
X₁₆ is Gly or Aib;
X₂₀ is Lys or Aib;
X₂₁ is Glu;
X₂₄ is Ala or Aib; and
X₂₇ is Met, Val, Leu or Nle.

13. (canceled)

14. The conjugate of claim 1 wherein
Y is selected from the group consisting of 3,5,3′,5′-tetra-iodothyronine and 3,5,3′-triiodo L-thyronine; and
Q is a GLP-1 agonist peptide comprising the sequence
HX₂EGTFTSDVSSYLEX₁₆QAAKEFIAWLVKG (SEQ ID NO: 45)
HAEGTFTSDVSSYLEGQAAKEFIAWLVKG (SEQ ID NO: 38) or
HAEGTFTSDVSSYLE(Aib)QAAKEFIAWLVKG (SEQ ID NO: 39), wherein
X₂ is D-serine or Aib; and
X₁₆ is Gly, Glu or Aib.

15. The conjugate of claim 14 wherein the GLP-1 agonist peptide further comprises a C-terminal extension of X₄₀,
(SEQ ID NO: 26)
GPSSGAPPPSX₄₀,
(SEQ ID NO: 27)
KRNRNNIAX₄₀
or
(SEQ ID NO: 28)
KRNRX₄₀
bound to amino acid 29 of the GLP-1 agonist peptide through a peptide bond, wherein X₄₀ is an amino acid selected from the group consisting of Cys or Lys.

16. (canceled)

17. The conjugate of claim 15 wherein the GLP-1 agonist peptide comprises a C-terminal extension of
(SEQ ID NO: 42)
GPSSGAPPPSK.

18. The conjugate of claim 15 wherein the thyroid hormone receptor ligand is covalently attached to the side chain amine of a Lys at position 30 or 40 of said C-terminal extension.

19. The conjugate of claim 17 wherein the thyroid hormone receptor ligand is covalently attached to the side chain amine of a Lys at position 40 of said C-terminal extension.

20. The conjugate of claim 19 wherein the GLP-1 agonist peptide comprises the sequence
(SEQ ID NO: 48)
HX2EGTFTSDVSSYLEX₁₆QAAKEFIAWLVKGGPSSGAPPPSK;
(SEQ ID NO: 49)
H(Aib)EGTFTSDVSSYLEEQAAKEFIAWLVKGGPSSGAPPPSK-amide;
(SEQ ID NO: 46)
HAEGTFTSDVSSYLEGQAAKEFIAWLVKGGPSSGAPPPSK
or
(SEQ ID NO: 47)
HAEGTFTSDVSSYLE(Aib)QAAKEFIAWLVKGGPSSGAPPPSK,
wherein
X₂ is D-serine or Aib; and
X₁₆ is Gly, Glu or Aib.

21. The conjugate of claim 20 wherein the thyroid hormone receptor ligand is covalently attached to the GLP-1 agonist peptide via an amino acid or dipeptide linker.

22. The conjugate of claim 21 wherein the thyroid hormone receptor ligand is 3,5,3′,5′-tetra-iodothyronine or 3,5,3′-triiodo L-thyronine, wherein the thyroid hormone receptor ligand is covalently linked to the side chain amine of a Lys of the GLP-1 agonist peptide through a gamma glutamic acid (γGlu) spacer added to the carboxylate of the thyroid hormone receptor.

23-27. (canceled)

28. The conjugate of claim 20, wherein L-Y comprises the structure: 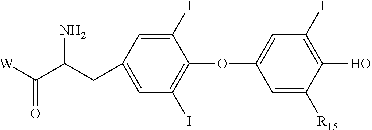
wherein
W is a bond, an amino acid, or dipeptide joining L-Y to Q; and
R₁₅ is H or I.

29. The conjugate of claim 28 wherein W is γ-Glu or the dipeptide, γ-Glu- γ-Glu.

30. A conjugate comprising the structure Q-L-Y;
wherein
Q is a GLP-1 agonist peptide comprising the sequence
(SEQ ID NO: 37)
X₁X₂EGTFTSDVSSYLX₁₅X₁₆QAAX₂₀X₂₁FIX₂₄WLX₂₇KGGPSSGAPPPSK;
or
(SEQ ID NO: 49)
H(aib)EGTFTSDVSSYLEEQAAKEFIAWLVKGGPSSGAPPPSK-amide
wherein
X₁ is selected from the group consisting of His, D-His, N-methyl-His, alpha-methyl-His, imidazole acetic acid, des-amino-His, hydroxyl-His, acetyl-His, homo-His, or alpha, alpha-dimethyl imidiazole acetic acid (DMIA);
X₂ is selected from the group consisting of Ser, D-Ser, Ala, D-Ala, Gly, N-methyl-Ser, Aib, Val, or α-amino-N-butyric acid;
X₁₅ is Asp, Glu, cysteic acid, homoglutamic acid or homocysteic acid;
X₁₆ is Gly, Glu, Lys or Aib,
X₂₀ is Glu, Ala, Lys or Aib;
X₂₁ is Glu or Aib;
X₂₄ is Ala, Glu, Lys or Aib; and
X₂₇ is Met, Val, Leu or Nle; and wherein L-Y comprises the structure 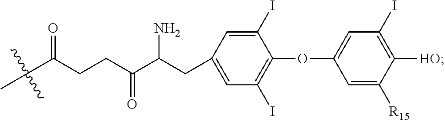
and
L-Y is conjugated to an amino acid side chain of the GLP-1 agonist peptide at position 40.

31-32. (canceled)

33. The conjugate of claim 30, wherein Q is a GLP-1 agonist peptide comprising the sequence
(SEQ ID NO: 37)
X₁X₂EGTFTSDVSSYLX₁₅X₁₆QAAX₂₀X₂₁FIX₂₄WLX₂₇KGGPSSGAPPPSK;
wherein
X₁ is His;
X₂ is selected from the group consisting of Ser, D-Ser, Ala, D-Ala, Gly, N-methyl-Ser, Aib, Val, or α-amino-N-butyric acid;
X₁₅ is Glu;
X₁₆ is Gly or Aib,
X₂₀ is Lys or Aib;
X₂₁ is Glu or Aib;
X₂₄ is Ala or Aib; and
X₂₇ is Met, Val, Leu or Nle.

34. (canceled)

35. A derivative of the conjugate of claim 1 further comprising the structure A-B, wherein
A is an amino acid or a hydroxy acid;
B is an N-alkylated amino acid linked to Q or Y through an amide bond between a carboxyl moiety of B and an amine of Q or Y; and
A-B comprises the structure: 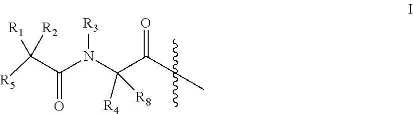
wherein
(a) R¹, R², R⁴ and R⁸ are independently selected from the group consisting of H, C1-C18 alkyl, C2-C18 alkenyl, (C1-C18 alkyl)OH, (C1-C18 alkyl)SH, (C2-C3 alkyl)SCH₃, (C1-C4 alkyl)CONH₂, (C1-C4 alkyl)COOH, (C1-C4 alkyl)NH₂, (C1-C4 alkyl)NHC(NH₂⁺)NH₂, (C0-C4 alkyl)(C3-C6 cycloalkyl), (C0-C4 alkyl)(C2-C5 heterocyclic), (C0-C4 alkyl)(C6-C10 aryl)R⁷, (C1-C4 alkyl)(C3-C9 heteroaryl), and C1-C12 alkyl(W1)C1-C12 alkyl, wherein W1 is a heteroatom selected from the group consisting of N, S and O, or
(ii) R¹ and R² together with the atoms to which they are attached form a C3-C12 cycloalkyl or aryl; or
(iii) R⁴ and R⁸ together with the atoms to which they are attached form a C3-C6 cycloalkyl;
(b) R³ is selected from the group consisting of C1-C18 alkyl, (C1-C18 alkyl)OH, (C1-C18 alkyl)NH₂, (C1-C18 alkyl)SH, (C0-C4 alkyl)(C3-C6)cycloalkyl, (C0-C4 alkyl)(C2-C5 heterocyclic), (C0-C4 alkyl)(C6-C10 aryl)R⁷, and (C1-C4 alkyl)(C3-C9 heteroaryl) or R⁴ and R³ together with the atoms to which they are attached form a 4, 5 or 6 member heterocyclic ring;
(c) R⁵ is NHR⁶ or OH;
(d) R⁶ is H, C₁-C₈ alkyl; and
(e) R⁷ is selected from the group consisting of H and OH
wherein the chemical cleavage half-life (t_{1/2}) of A-B from Q or Y is at least about 1 hour to about 1 week in PBS under physiological conditions.

36-37. (canceled)

38. The conjugate of claim 1, further comprising an amino acid side chain on Q, at a position corresponding to position 10, 20, or 24 of native glucagon, or at position 30, 37, 38, 39, 40, 41, 32, or 43 of a C-terminal extended glucagon analog, or the C-terminal amino acid, covalently attached to an acyl group or an alkyl group via an alkyl amine, amide, ether, ester, thioether, or thioester linkage, which acyl group or alkyl group is non-native to a naturally occurring amino acid.

39-42. (canceled)

43. A pharmaceutical composition comprising the conjugate of claim 1, and a pharmaceutically acceptable carrier.

44. A method for treating a disease or medical condition in a patient, wherein the disease or medical condition is selected from the group consisting of hyperlipidemia, metabolic syndrome, diabetes, obesity, liver steatosis, and chronic cardiovascular disease, comprising administering to the patient the pharmaceutical composition of claim 43 in an amount effective to treat the disease or medical condition.
